SEQUENCE LISTING (150 sequences)

SEQ ID NO: 1 - protein, 5 aa, Artificial Sequence
  Synthetic: 7A3 VH CDR1 (Kabat)
GYTIH

SEQ ID NO: 2 - protein, 7 aa, Artificial Sequence
  Synthetic: 7A3 VH CDR1 (Chothia)
GYSFTGY

SEQ ID NO: 3 - protein, 17 aa, Artificial Sequence
  Synthetic: 7A3 VH CDR2 (Kabat)
LINPYTGGTDYNQKFKG

SEQ ID NO: 4 - protein, 6 aa, Artificial Sequence
  Synthetic: 7A3 VH CDR2 (Chothia)
NPYTGG

SEQ ID NO: 5 - protein, 16 aa, Artificial Sequence
  Synthetic: 7A3 VH CDR3
ERPGGSQFIYYYPMDY

SEQ ID NO: 6 - protein, 11 aa, Artificial Sequence
  Synthetic: 7A3 VH CDR1
RASANIYSYLA

SEQ ID NO: 7 - protein, 7 aa, Artificial Sequence
  Synthetic: 7A3 VH CDR2
NAITLAE

SEQ ID NO: 8 - protein, 9 aa, Artificial Sequence
  Synthetic: 7A3 VH CDR3
QHHYGTPFT

SEQ ID NO: 9 - protein, 125 aa, Artificial Sequence
  Synthetic: 7A3 VH Hu-VH2
QVQLVQSGAEVKKPGASVKVSCKASGYSFTGYTMHWVRQAPGQGLEWMGLINPYTGGTDYNQKFQGRVTMTVDKSTSTAYMELSSLRSEDTAVYYCARERPGGSQFIYYYPMDYWGQGTTVTVSS

SEQ ID NO: 10 - protein, 107 aa, Artificial Sequence
  Synthetic: 7A3 VH Hu-VL1
DIQMTQSPSSLSASVGDRVTITCRASANIYSYLAWYQQKPGKAPKFLVYNAITLAEGVPSRFSGSGSGTDFTLTISSLQPEDFATYYCQHHYGTPFTFGGGTKLEIK

SEQ ID NO: 11 - protein, 5 aa, Artificial Sequence
  Synthetic: 7B1 VH CDR1 (Kabat)
GYTMH

SEQ ID NO: 12 - protein, 7 aa, Artificial Sequence
  Synthetic: 7B1 VH CDR1 (Chothia)
GYSFTGY

SEQ ID NO: 13 - protein, 17 aa, Artificial Sequence
  Synthetic: 7B1 VH CDR2 (Kabat)
LINPYTGGTDYNQKFKG

SEQ ID NO: 14 - protein, 6 aa, Artificial Sequence
  Synthetic: 7B1 VH CDR2 (Chothia)
NPYTGG

SEQ ID NO: 15 - protein, 16 aa, Artificial Sequence
  Synthetic: 7B1 VH CDR3
ERPGGSQFIYYYALDY

SEQ ID NO: 16 - protein, 11 aa, Artificial Sequence
  Synthetic: 7B1 VH CDR1
RASENIYSYLA

SEQ ID NO: 17 - protein, 7 aa, Artificial Sequence
  Synthetic: 7B1 VH CDR2
NADTLAE

SEQ ID NO: 18 - protein, 9 aa, Artificial Sequence
  Synthetic: 7B1 VH CDR3
QHHYGTPFT

SEQ ID NO: 19 - protein, 125 aa, Artificial Sequence
  Synthetic: 7B1 VH Hu-VH2
QVQLVQSGAEVKKPGASVKVSCKASGYSFTGYTMHWVRQAPGQGLEWMGLINPYTGGTDYNQKFQGRVTMTVDRSTSTAYMELSSLRSEDTAVYYCARERPGGSQFIYYYALDYWGQGTTVTVSS

SEQ ID NO: 20 - protein, 107 aa, Artificial Sequence
  Synthetic: 7B1 VH Hu-VL1
DIQMTQSPSSLSASVGDRVTITCRASENIYSYLAWYQQKPGKAPKFLVYNADTLAEGVPSRFSGSGSGTDFTLTISSLQPEDFATYYCQHHYGTPFTFGGGTKLEIK

SEQ ID NO: 21 - protein, 5 aa, Artificial Sequence
  Synthetic: VH CDR1 Consensus
  misc_feature (4)..(4): X is I or M
GYTXH

SEQ ID NO: 22 - protein, 16 aa, Artificial Sequence
  Synthetic: VH CDR3 Consensus
  misc_feature (13)..(13): X is P or A
  misc_feature (14)..(14): X is M or L
ERPGGSQFIYYYXXDY

SEQ ID NO: 23 - protein, 11 aa, Artificial Sequence
  Synthetic: VH CDR1 Consensus
  misc_feature (4)..(4): X is A or E
RASXNIYSYLA

SEQ ID NO: 24 - protein, 7 aa, Artificial Sequence
  Synthetic: VH CDR2 Consensus
  misc_feature (3)..(3): X is I or D
NAXTLAE

SEQ ID NO: 25 - protein, 454 aa, Artificial Sequence
  Synthetic: 7A3 AQQ HC amino acids
QVQLVQSGAEVKKPGASVKVSCKASGYSFTGYTMHWVRQAPGQGLEWMGLINPYTGGTDYNQKFQGRVTMTVDKSTSTAYMELSSLRSEDTAVYYCARERPGGSQFIYYYPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 214 aa, Artificial Sequence
  Synthetic: 7A3 LC amino acids
DIQMTQSPSSLSASVGDRVTITCRASANIYSYLAWYQQKPGKAPKFLVYNAITLAEGVPSRFSGSGSGTDFTLTISSLQPEDFATYYCQHHYGTPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 454 aa, Artificial Sequence
  Synthetic: 7B1 AQQ HC amino acids
QVQLVQSGAEVKKPGASVKVSCKASGYSFTGYTMHWVRQAPGQGLEWMGLINPYTGGTDYNQKFQGRVTMTVDRSTSTAYMELSSLRSEDTAVYYCARERPGGSQFIYYYALDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 214 aa, Artificial Sequence
  Synthetic: 7B1 LC amino acids
DIQMTQSPSSLSASVGDRVTITCRASENIYSYLAWYQQKPGKAPKFLVYNADTLAEGVPSRFSGSGSGTDFTLTISSLQPEDFATYYCQHHYGTPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 5 aa, Artificial Sequence
  Synthetic: 7H7 VH CDR1
TSWMS

SEQ ID NO: 30 - protein, 17 aa, Artificial Sequence
  Synthetic: 7H7 VH CDR2
NIKQDGSEKYYVDSVKG

SEQ ID NO: 31 - protein, 10 aa, Artificial Sequence
  Synthetic: 7H7 VH CDR3
DRPVAGASAL

SEQ ID NO: 32 - protein, 11 aa, Artificial Sequence
  Synthetic: 7H7 VL CDR1
RASQSISGWLA

SEQ ID NO: 33 - protein, 7 aa, Artificial Sequence
  Synthetic: 7H7 VL CDR2
KASSLES

SEQ ID NO: 34 - protein, 9 aa, Artificial Sequence
  Synthetic: 7H7 VL CDR3
QQYYGSSRT

SEQ ID NO: 35 - protein, 5 aa, Artificial Sequence
  Synthetic: 1B3 VH CDR1
TSWMS

SEQ ID NO: 36 - protein, 17 aa, Artificial Sequence
  Synthetic: 1B3 VH CDR2
NIKQDGSEKYYVDSVKG

SEQ ID NO: 37 - protein, 10 aa, Artificial Sequence
  Synthetic: 1B3 VH CDR3
DRPVAGASAL

SEQ ID NO: 38 - protein, 11 aa, Artificial Sequence
  Synthetic: 1B3 VL CDR1
RASQSISGWLA

SEQ ID NO: 39 - protein, 7 aa, Artificial Sequence
  Synthetic: 1B3 VL CDR2
KASSLES

SEQ ID NO: 40 - protein, 9 aa, Artificial Sequence
  Synthetic: 1B3 VL CDR3
QQYYGSSRT

SEQ ID NO: 41 - protein, 5 aa, Artificial Sequence
  Synthetic: 3B6 VH CDR1
TYWMS

SEQ ID NO: 42 - protein, 17 aa, Artificial Sequence
  Synthetic: 3B6 VH CDR2
NIKQDGSEKYYVDSVKG

SEQ ID NO: 43 - protein, 10 aa, Artificial Sequence
  Synthetic: 3B6 VH CDR3
DRPVAGASAL

SEQ ID NO: 44 - protein, 11 aa, Artificial Sequence
  Synthetic: 3B6 VL CDR1
RASQSISGWLA

SEQ ID NO: 45 - protein, 7 aa, Artificial Sequence
  Synthetic: 3B6 VL CDR2
KASSLES

SEQ ID NO: 46 - protein, 9 aa, Artificial Sequence
  Synthetic: 3B6 VL CDR3
QQYYGSSRT

SEQ ID NO: 47 - protein, 5 aa, Artificial Sequence
  Synthetic: 8B1 VH CDR1
THWMS

SEQ ID NO: 48 - protein, 17 aa, Artificial Sequence
  Synthetic: 8B1 VH CDR2
NIKQDGSEKYYVDSVKG

SEQ ID NO: 49 - protein, 10 aa, Artificial Sequence
  Synthetic: 8B1 VH CDR3
DRPVAGASAL

SEQ ID NO: 50 - protein, 11 aa, Artificial Sequence
  Synthetic: 8B1 VL CDR1
RASQSISGWLA

SEQ ID NO: 51 - protein, 7 aa, Artificial Sequence
  Synthetic: 8B1 VL CDR2
KASSLES

SEQ ID NO: 52 - protein, 9 aa, Artificial Sequence
  Synthetic: 8B1 VL CDR3
QQYYGSSRT

SEQ ID NO: 53 - protein, 5 aa, Artificial Sequence
  Synthetic: 4A3 VH CDR1
SSWMS

SEQ ID NO: 54 - protein, 17 aa, Artificial Sequence
  Synthetic: 4A3 VH CDR2
NIKQDGSEKYYVDSVKG

SEQ ID NO: 55 - protein, 10 aa, Artificial Sequence
  Synthetic: 4A3 VH CDR3
DRPVAGASAL

SEQ ID NO: 56 - protein, 11 aa, Artificial Sequence
  Synthetic: 4A3 VL CDR1
RASQSISGWLA

SEQ ID NO: 57 - protein, 7 aa, Artificial Sequence
  Synthetic: 4A3 VL CDR2
KASSLES

SEQ ID NO: 58 - protein, 9 aa, Artificial Sequence
  Synthetic: 4A3 VL CDR3
QQYYGSSRT

SEQ ID NO: 59 - protein, 5 aa, Artificial Sequence
  Synthetic: 9H9 VH CDR1
TYWMS

SEQ ID NO: 60 - protein, 17 aa, Artificial Sequence
  Synthetic: 9H9 VH CDR2
NIKQDGSEKYYVDSVKG

SEQ ID NO: 61 - protein, 10 aa, Artificial Sequence
  Synthetic: 9H9 VH CDR3
DRPVAGASAL

SEQ ID NO: 62 - protein, 11 aa, Artificial Sequence
  Synthetic: 9H9 VL CDR1
RASQSISGWLA

SEQ ID NO: 63 - protein, 7 aa, Artificial Sequence
  Synthetic: 9H9 VL CDR2
KASSLES

SEQ ID NO: 64 - protein, 9 aa, Artificial Sequence
  Synthetic: 9H9 VL CDR3
QQYYGSSRT

SEQ ID NO: 65-76 - skipped (12 entries)

SEQ ID NO: 77 - protein, 119 aa, Artificial Sequence
  Synthetic: 7H7 VH without signal sequence
EVQLVESGGGLVQPGGSLRLSCAASGGTISTSWMSWVRQAPGKGLEWVANIKQDGSEKYYVDSVKGRFTISRDNAKNSLYLQMNSLRVEDTAIYYCARDRPVAGASALWGQGTLVTVSS

SEQ ID NO: 78 - protein, 107 aa, Artificial Sequence
  Synthetic: 7H7 VL without signal sequence
DIQMTQSPSTLSASVGDRVTITCRASQSISGWLAWYQQKQGKAPKLLIYKASSLESGVPSRFSGSGSGTEFTLTISSLQPDDFATYYCQQYYGSSRTFGQGTNVEIK

SEQ ID NO: 79 - protein, 119 aa, Artificial Sequence
  Synthetic: 1B3 VH without signal sequence
EVQLVESGGGLVQPGGSLRLSCAASGGTISTSWMSWVRQAPGKGLEWVANIKQDGSEKYYVDSVKGRFTISRDNAKNSLYLQMNSLRVEDTAMYYCARDRPVAGASALWGQGTLVTVSS

SEQ ID NO: 80 - protein, 107 aa, Artificial Sequence
  Synthetic: 1B3 VL without signal sequence
DIQMTQSPSTLSASVGDRVTITCRASQSISGWLAWYQQKPGKAPKLLIYKASSLESGVPSRFSGSGSGTEFTLTISSLQPDDFATYYCQQYYGSSRTFGQGTNVEIK

SEQ ID NO: 81 - protein, 119 aa, Artificial Sequence
  Synthetic: 3B6 VH without signal sequence
EVQLVESGGGLVQPGGSLRLSCAASGGTTSTYWMSWVRQAPGKGLEWVANIKQDGSEKYYVDSVKGRFTISRDNAKNSLNLQMNSLRVEDTAIYYCARDRPVAGASALWGQGTLVTVSS

SEQ ID NO: 82 - protein, 107 aa, Artificial Sequence; identical to SEQ ID NO: 80
  Synthetic: 3B6 VL without signal sequence

SEQ ID NO: 83 - protein, 119 aa, Artificial Sequence
  Synthetic: 8B1 VH without signal sequence
EVRLVESGGGLVQPGGSLRLSCAASGDIISTHWMSWVRQAPGKGLEWVANIKQDGSEKYYVDSVKGRFTISRDNAKNSLYLQMNTLRVEDTAIYYCTRDRPVAGASALWGQGTLVTVSS

SEQ ID NO: 84 - protein, 107 aa, Artificial Sequence
  Synthetic: 8B1 VL without signal sequence
DIQMTQSPSTLSASVGDRVTITCRASQSISGWLAWYQQKPGKAPKLLIYKASSLESGVPLRFSGSGSGTEFTLTISSLQPDDFATYYCQQYYGSSRTFGQGTNVEIK

SEQ ID NO: 85 - protein, 119 aa, Artificial Sequence
  Synthetic: 4A3 VH without signal sequence
EVQLVESGGGLVQPGGSLRLSCAASGGIISSSWMSWVRQAPGKGLEWVANIKQDGSEKYYVDSVKGRFTISRDNAKDLLYLQMNSLRVEDTALYYCARDRPVAGASALWGQGTLVTVSS

SEQ ID NO: 86 - protein, 107 aa, Artificial Sequence; identical to SEQ ID NO: 80
  Synthetic: 4A3 VL without signal sequence

SEQ ID NO: 87 - protein, 119 aa, Artificial Sequence
  Synthetic: 9H9 VH without signal sequence
EVQLVESGGGLVQPGGSLRLSCAASGGIISTYWMSWVRQAPGKGLEWVANIKQDGSEKYYVDSVKGRFTISRDNAKNSLYLQMNSLRVEDTAMYYCARDRPVAGASALWGQGTLVTVSS

SEQ ID NO: 88 - protein, 107 aa, Artificial Sequence; identical to SEQ ID NO: 80
  Synthetic: 9H9 VL without signal sequence

SEQ ID NO: 89 - protein, 598 aa, Artificial Sequence
  Synthetic: ILT4 Isoform 1(Q8N423-1)
MTPIVTVLICLGLSLGPRTHVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEEEHPQCLNSQPHARGSSRAIFSVGPVSPNRRWSHRCYGYDLNSPYVWSSPSDLLELLVPGVSKKPSLSVQPGPVVAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 90 - DNA, 1362 bp, Artificial Sequence
  Synthetic: 7A3 AQQ HC DNA
caagtgcagctggtgcagagcggcgctgaggtgaagaagcccggcgccagcgtcaaggtcagctgtaaggccagcggctacagcttcactggctacactatgcactgggtgagacaagcccccggccaaggactggagtggatgggactgatcaacccttacactggcggcactgactacaaccagaaattccaaggaagggtcacaatgactgtggacaagagcacatccactgcctacatggagctgagctctctgaggagcgaggacacagccgtgtactactgtgccagagagagacccggcggcagccagttcatctactactaccctatggactactggggccaaggcacaacagtgactgtgagcagcgctagcacaaagggcccttccgtgtttccactggctcccagctctaagtccaccagcggaggaacagccgctctgggctgtctggtgaaggactatttcccagagcccgtgaccgtgagctggaactctggcgccctgaccagcggagtgcatacatttcctgctgtgctgcagtccagcggcctgtactctctgtcttccgtggtgaccgtgccaagctcttccctgggcacccagacatatatctgcaacgtgaatcacaagccatccaatacaaaggtggacaagaaggtggagcccaagagctgtgataagacccatacatgccccccttgtcctgctccagaggctcagggaggaccatccgtgttcctgtttccacccaagcctaaggacaccctgatgatctctaggaccccagaggtgacctgcgtggtggtggacgtgagccacgaggatcccgaggtgaagttcaactggtacgtggatggcgtggaggtgcataatgccaagacaaagccaagggaggagcagtacaatagcacctatcgggtggtgtctgtgctgacagtgctgcaccaggactggctgaacggcaaggagtacaagtgccaggtgtctaataaggccctgcccgctcctatcgagaagaccatctccaaggccaagggccagcctagggagccacaggtgtacacactgcctccaagccgggacgagctgaccaagaaccaggtgtctctgacatgtctggtgaagggcttctatccctctgatatcgctgtggagtgggagtccaatggccagcctgagaacaattacaagaccacaccccctgtgctggactccgatggcagcttctttctgtattccaagctgaccgtggataagagcaggtggcagcagggcaacgtgttttcttgttccgtgatgcatgaggctctgcacaatcattacacacagaagagcctgtctctgtcccctggc

SEQ ID NO: 91 - DNA, 642 bp, Artificial Sequence
  Synthetic: 7A3 LC DNA
gacatccagatgacacagagcccaagctctctgagcgccagcgtgggagatagggtgacaatcacatgtagggccagcgccaacatctacagctatctggcttggtaccagcagaagcccggcaaggcccctaagtttctggtgtacaacgccatcactctggctgagggcgtgcctagcagatttagcggcagcggaagcggcacagacttcactctgacaatcagctctctgcagccagaggatttcgccacatactactgccagcaccactacggcactcctttcacattcggcggcggcactaagctggagatcaagcgtacggtggccgctccaagcgtgttcatctttcccccttctgacgagcagctgaagtctggcacagcctccgtggtgtgcctgctgaacaacttctaccccagagaggccaaggtgcagtggaaggtggataacgctctgcagtctggcaattcccaggagagcgtgaccgagcaggactctaaggattccacatatagcctgagctctaccctgacactgtctaaggccgattacgagaagcacaaggtgtatgcttgcgaggtgacccatcagggcctgtccagcccagtgacaaagtccttcaatcgcggcgagtgt

SEQ ID NO: 92 - DNA, 1362 bp, Artificial Sequence
  Synthetic: 7B1 AQQ HC DNA
caagtgcagctggtgcagagcggcgctgaggtgaagaagcccggcgccagcgtgaaagtgagctgtaaggcctccggctacagcttcactggctacactatgcactgggtcagacaagcccccggccaaggactggagtggatgggactgatcaacccttacactggcggcactgactacaaccagaagttccaaggaagggtgactatgactgtggataggtccacaagcacagcctacatggagctgtcctctctgagatccgaggacactgccgtgtactactgtgctagggagagacccggcggcagccagttcatctactactacgctctggactactggggccaaggcacaacagtcacagtgagcagcgctagcacaaagggcccttccgtgtttccactggctcccagctctaagtccaccagcggaggaacagccgctctgggctgtctggtgaaggactatttcccagagcccgtgaccgtgagctggaactctggcgccctgaccagcggagtgcatacatttcctgctgtgctgcagtccagcggcctgtactctctgtcttccgtggtgaccgtgccaagctcttccctgggcacccagacatatatctgcaacgtgaatcacaagccatccaatacaaaggtggacaagaaggtggagcccaagagctgtgataagacccatacatgccccccttgtcctgctccagaggctcagggaggaccatccgtgttcctgtttccacccaagcctaaggacaccctgatgatctctaggaccccagaggtgacctgcgtggtggtggacgtgagccacgaggatcccgaggtgaagttcaactggtacgtggatggcgtggaggtgcataatgccaagacaaagccaagggaggagcagtacaatagcacctatcgggtggtgtctgtgctgacagtgctgcaccaggactggctgaacggcaaggagtacaagtgccaggtgtctaataaggccctgcccgctcctatcgagaagaccatctccaaggccaagggccagcctagggagccacaggtgtacacactgcctccaagccgggacgagctgaccaagaaccaggtgtctctgacatgtctggtgaagggcttctatccctctgatatcgctgtggagtgggagtccaatggccagcctgagaacaattacaagaccacaccccctgtgctggactccgatggcagcttctttctgtattccaagctgaccgtggataagagcaggtggcagcagggcaacgtgttttcttgttccgtgatgcatgaggctctgcacaatcattacacacagaagagcctgtctctgtcccctggc

SEQ ID NO: 93 - DNA, 642 bp, Artificial Sequence
  Synthetic: 7B1 LC DNA
gacatccagatgacacagagcccaagctctctgagcgccagcgtgggagatagggtgacaatcacttgtagggccagcgagaacatctacagctatctggcttggtaccagcagaagcccggcaaggccccaaagtttctggtgtacaacgccgacactctcgctgagggcgtcccttctaggttttccggcagcggctccggcactgacttcacactgactatcagctctctgcagccagaggatttcgccacatactactgccagcaccactacggcactcctttcacattcggcggcggcactaagctggagatcaagcgtacggtggccgctccaagcgtgttcatctttcccccttctgacgagcagctgaagtctggcacagcctccgtggtgtgcctgctgaacaacttctaccccagagaggccaaggtgcagtggaaggtggataacgctctgcagtctggcaattcccaggagagcgtgaccgagcaggactctaaggattccacatatagcctgagctctaccctgacactgtctaaggccgattacgagaagcacaaggtgtatgcttgcgaggtgacccatcagggcctgtccagcccagtgacaaagtccttcaatcgcggcgagtgt

SEQ ID NO: 94-96 - skipped (3 entries)

SEQ ID NO: 97 - protein, 125 aa, Artificial Sequence
  Synthetic: 7A3 hu-VH1
EVQLVQSGAEVKKPGASVKVSCKASGYSFTGYTIHWVRQAPGQGLEWIGLINPYTGGTDYNQKFQGRVTLTVDKSTSTAYMELSSLRSEDTAVYYCARERPGGSQFIYYYPMDYWGQGTTVTVSS

SEQ ID NO: 98 - protein, 125 aa, Artificial Sequence
  Synthetic: 7A3 hu-VH3
EVQLVQSGSELKKPGASVKVSCKASGYSFTGYTIHWVRQAPGQGLEWIGLINPYTGGTDYNQGFTGRFVLSVDKSVSTAYLQISSLKAEDTAVYYCARERPGGSQFIYYYPMDYWGQGTTVTVSS

SEQ ID NO: 99 - protein, 125 aa, Artificial Sequence
  Synthetic: 7A3 hu-VH4
QVQLVQSGSELKKPGASVKVSCKASGYSFTGYTMNWVRQAPGQGLEWMGLINPYTGGTDYNQGFTGRFVFSVDKSVSTAYLQISSLKAEDTAVYYCARERPGGSQFIYYYPMDYWGQGTTVTVSS

SEQ ID NO: 100 - protein, 107 aa, Artificial Sequence
  Synthetic: 7A3 hu-VL2
DIQMTQSPSSLSASVGDRVTITCRASANIYSYLAWYQQKPGKAPKFLVYNAITLASGVPSRFSGSGSGTDFTLTISSLQPEDFATYYCQHHYGTPFTFGGGTKLEIK

SEQ ID NO: 101 - protein, 107 aa, Artificial Sequence
  Synthetic: 7A3 hu-VL3
DIVMTQSPATLSLSPGERATLSCRASANIYSYLAWYQQKPGQAPRFLVYNAITLAEGVPARFSGSGSGTDFTLTISSLEPEDFAVYYCQHHYGTPFTFGGGTKLEIK

SEQ ID NO: 102 - protein, 107 aa, Artificial Sequence
  Synthetic: 7A3 hu-VL4
EIVMTQSPATLSLSPGERATLSCRASANIYSYLAWYQQKPGQAPRFLVYNAITRATGIPARFSGSGSGTDFTLTISSLEPEDFAVYYCQHHYGTPFTFGGGTKLEIK

SEQ ID NO: 103 - protein, 125 aa, Artificial Sequence
  Synthetic: 7B1 hu-VH1
EVQLVQSGAEVKKPGASVKVSCKASGYSFTGYTMHWVRQAPGQGLEWIGLINPYTGGTDYNQKFQGRVTLTVDRSTSTAYMELSSLRSEDTAVYYCARERPGGSQFIYYYALDYWGQGTTVTVSS

SEQ ID NO: 104 - protein, 125 aa, Artificial Sequence
  Synthetic: 7B1 hu-VH3
EVQLVQSGSELKKPGASVKVSCKASGYSFTGYTMHWVRQAPGQGLEWIGLINPYTGGTDYNQGFTGRFVLSVDRSVSTAYLQISSLKAEDTAVYYCARERPGGSQFIYYYALDYWGQGTTVTVSS

SEQ ID NO: 105 - protein, 125 aa, Artificial Sequence
  Synthetic: 7B1 hu-VH4
QVQLVQSGSELKKPGASVKVSCKASGYSFTGYTMNWVRQAPGQGLEWMGLINPYTGGTDYNQGFTGRFVFSVDRSVSTAYLQISSLKAEDTAVYYCARERPGGSQFIYYYALDYWGQGTTVTVSS

SEQ ID NO: 106 - protein, 107 aa, Artificial Sequence
  Synthetic: 7B1 hu-VL2
DIQMTQSPSSLSASVGDRVTITCRASENIYSYLAWYQQKPGKAPKFLVYNADTLASGVPSRFSGSGSGTDFTLTISSLQPEDFATYYCQHHYGTPFTFGGGTKLEIK

SEQ ID NO: 107 - protein, 107 aa, Artificial Sequence
  Synthetic: 7B1 hu-VL3
DIVMTQSPATLSLSPGERATLSCRASENIYSYLAWYQQKPGQAPRFLVYNADTLAEGVPARFSGSGSGTDFTLTISSLEPEDFAVYYCQHHYGTPFTFGGGTKLEIK

SEQ ID NO: 108 - protein, 107 aa, Artificial Sequence
  Synthetic: 7B1 hu-VL4
EIVMTQSPATLSLSPGERATLSCRASENIYSYLAWYQQKPGQAPRFLVYNADTRATGIPARFSGSGSGTDFTLTISSLEPEDFAVYYCQHHYGTPFTFGGGTKLEIK

SEQ ID NO: 109 - protein, 144 aa, Artificial Sequence
  Synthetic: 7A3 VH
MGWSWIFLFLLSGTAGVHSEVQLQQSGPELAKPGASMKISCRASGYSFTGYTIHWVKQSHGKNLEWIGLINPYTGGTDYNQKFKGKATLTVDKSSSTAYMDLLSLTSEDSAVYYCARERPGGSQFIYYYPMDYWGQGTSVTVSS

SEQ ID NO: 110 - protein, 127 aa, Artificial Sequence
  Synthetic: 7A3 VL
MSVPTQVLGLLLLWLTGVRCDIQMTQSPASLSVSVGETVTITCRASANIYSYLAWYQQKQGKSPQFLVYNAITLAEGVPSRFSGSGSGTQFSLKINSLQPEDFGSYYCQHHYGTPFTFGSGTKLEIK

SEQ ID NO: 111 - DNA, 432 bp, Artificial Sequence
  Synthetic: 7A3 VH
atgggatggagctggatctttctcttcctcctgtcaggaactgcaggtgtccactctgaggtccagctgcaacagtctggacctgagctggcgaagcctggagcttcaatgaagatatcctgcagggcttctggttactcattcactggctacaccatccactgggtgaagcagagccatggaaagaaccttgagtggattggacttattaatccttacactggtggtactgactacaaccagaagttcaagggcaaggccacattaactgtcgacaagtcatccagcacagcctacatggacctcctcagtctaacatctgaagactctgcagtctactactgtgcaagggagcgccccggggggtcccaatttatatattattatcctatggactactggggtcaaggaacctcggtcaccgtctcctca

SEQ ID NO: 112 - DNA, 381 bp, Artificial Sequence
  Synthetic: 7A3 VL
atgagtgtgcccactcaggtcctggggttgctgctgctgtggcttacaggtgtcagatgtgacatccagatgactcagtctccagcctccctatctgtatctgtgggagaaactgtcaccatcacatgtcgagcaagtgcgaatatttacagttatttagcatggtatcagcagaaacagggaaaatctcctcagttcctggtctataatgcaataaccttagcagaaggtgtgccatcaaggttcagtggcagtggatcaggcacacagttttctctgaagatcaacagcctgcagcctgaagattttgggagttattactgtcaacatcattatggtactccattcacgttcggctcggggacaaagttggagataaaa

SEQ ID NO: 113 - protein, 144 aa, Artificial Sequence
  Synthetic: 7B1 VH
MGWSWIFLFLLSGTAGVHSEVQLQQSGPELAKPGASMKISCKASGYSFTGYTMHWVKQSHGKNLEWIGLINPYTGGTDYNQKFKGKATLTVDRSSSTAYMDLLSLTSEDSAVYYCARERPGGSQFIYYYALDYWGQGTSVTVSS

SEQ ID NO: 114 - protein, 127 aa, Artificial Sequence
  Synthetic: 7B1 VL
MSVPTQVLGLLLLWLTGARCDIQMTQSPASLSVSVGETVTITCRASENIYSYLAWYQQKQGRSPQFLVYNADTLAEGVPSRFSGSGSGTQFSLKINSLQPEDFGSYYCQHHYGTPFTFGSGTKLEIK

SEQ ID NO: 115 - DNA, 432 bp, Artificial Sequence
  Synthetic: 7B1 VH
atgggatggagctggatctttctcttcctcctgtcaggaactgcaggtgtccactctgaggtccagctgcaacagtctggacctgagctggcgaagcctggagcttcaatgaagatatcctgtaaggcttctggttactcattcactggctacaccatgcactgggtgaagcagagccatggaaagaaccttgagtggattggactaattaatccttacactggtggtactgactacaaccagaagttcaagggcaaggccacattaactgtcgacaggtcttccagcacagcctacatggacctcctcagtctgacatctgaggactctgcagtctattactgtgcaagggagcgccccggggggtcccaatttatatattactatgctctggactactggggtcaaggaacctcggtcaccgtctcctca

SEQ ID NO: 116 - DNA, 381 bp, Artificial Sequence
  Synthetic: 7B1 VL
atgagtgtgcccactcaggtcctggggttgctgctgctgtggcttacaggtgccagatgtgacatccagatgactcagtctccagcctccctatctgtatctgtgggagaaactgtcaccatcacatgtcgagcaagtgagaatatttacagttatttagcatggtatcagcagaaacagggaagatctcctcagttcctggtctataatgcagataccttagcggaaggtgtgccatcaaggttcagtggcagtggatcaggcacacagttttctctaaagatcaacagcctacagcctgaagattttgggagttattactgtcaacatcattatggtactccattcacgttcggctcggggacaaagttggagataaaa

SEQ ID NO: 117 - DNA, 414 bp, Artificial Sequence
  Synthetic: 7H7 VH DNA Sequence with signal sequence
  misc_feature (21)..(21): n is a, c, g, or t
atggaattggggctgagctgngttttccttgttgctattttagaaggtgtccagtgtgaggtgcaactggtggagtctgggggaggcttggtccagcctggggggtccctgagactctcctgtgcagcctctggaggcaccattagtacctcttggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaggacacggctatatattactgtgcgagagatcgtccagtggctggtgcgtcggccctctggggccagggaaccctggtcaccgtctcctca

SEQ ID NO: 118 - DNA, 381 bp, Artificial Sequence
  Synthetic: 7H7 VL DNA Sequence with signal sequence
atgagggtccccgctcagctcctggggctcctgctgctctggctcccaggtgccaaatgtgacatccagatgacccagtctccttccaccctgtctgcatctgtaggagacagagtcaccatcacttgccgggccagtcagagtataagtggctggttggcctggtatcagcagaaacaagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtattatggttcttctcggacgttcggccaagggaccaatgtggaaatcaaa

SEQ ID NO: 119 - DNA, 414 bp, Artificial Sequence
  Synthetic: 1B3 VH DNA Sequence with signal sequence
atggaattggggctgagctgggttttccttgttgctattttagaaggtgtccagtgtgaggtgcaactggtggagtctgggggaggcttggtccagcctggggggtccctgagactctcctgtgcagcctctggaggcaccattagtacctcttggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaagacacggctatgtattactgtgcgagagatcgtccagtggctggtgcgtcggccctctggggccagggaaccctggtcaccgtctcctca

SEQ ID NO: 120 - DNA, 381 bp, Artificial Sequence
  Synthetic: 1B3 VL DNA Sequence with signal sequence
atgagggtccccgctcagctcctggggctcctgctgctctggctcccaggtgccaaatgtgacatccagatgacccagtctccttccaccctgtctgcatctgtaggagacagagtcaccatcacttgccgggccagtcagagtattagtggctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtattatggttcttctcggacgttcggccaagggaccaatgtggaaatcaaa

SEQ ID NO: 121 - DNA, 414 bp, Artificial Sequence
  Synthetic: 3B6 VH DNA Sequence with signal sequence
atggaattggggctgagctgggttttccttgttgctattttagaaggtgtccagtgtgaggtgcaactggtggagtctgggggaggcttggtccagcctggggggtccctgagactctcctgtgcagcctctggaggcacaaccagtacctattggatgagctgggtccgccaggctccagggaaggggctggaatgggtggccaacataaagcaagatggaagtgagaaatattatgtggactctgtgaagggccgattcaccatctccagagacaacgccaagaactcactgaatttgcaaatgaacagcctgagagtcgaggacacggctatatattactgtgcgagagatcgtccagtggctggtgcgtcggccctctggggccagggaaccctggtcaccgtctcctca

SEQ ID NO: 122 - DNA, 381 bp, Artificial Sequence; identical to SEQ ID NO: 120
  Synthetic: 3B6 VL DNA Sequence with signal sequence

SEQ ID NO: 123 - DNA, 414 bp, Artificial Sequence
  Synthetic: 8B1 VH DNA Sequence with signal sequence
atggaattggggctgagctgggttttccttgttgctattttagaaggtgtcaagtgtgaggtgcgactggtggagtctgggggaggcttggtccagcctggggggtccctgagactctcctgtgcagcctctggagacataattagtacccattggatgagctgggtccgccaggctccagggaaggggctggaatgggtggccaacataaaacaagatggaagtgagaagtattatgtggactctgtgaagggccgattcaccatctccagagacaacgccaagaactcactgtatttgcaaatgaacaccctgagagtcgaggacacggctatatattactgtacgagagatcgtccagtggctggtgcgtcggccctctggggccagggaaccctggtcaccgtctcctca

SEQ ID NO: 124 - DNA, 381 bp, Artificial Sequence
  Synthetic: 8B1 VL DNA Sequence with signal sequence
atgagggtccccgctcagctcctggggctcctgctgctctggctcccaggtgccaaatgtgacatccagatgacccagtctccttccaccctgtctgcatctgtaggagacagagtcaccatcacttgccgggccagtcagagtattagtggctggttggcctggtatcagcagaaaccagggaaagcccctaagctcctgatctataaggcgtctagtttagaaagtggggtcccattaaggttcagcggcagtggatctgggacagaattcactctcaccatcagcagcctgcagcctgatgattttgcaacttattactgccaacagtattatggttcttctcggacgttcggccaagggaccaatgtggaaatcaaa

SEQ ID NO: 125 - DNA, 414 bp, Artificial Sequence
  Synthetic: 4A3 VH DNA Sequence with signal sequence
atggaattggggctgagctgggttttccttgttgctattttagaaggtgtccagtgtgaggtgcaactggtggagtctgggggaggcttggtccagccgggggggtccctgagactctcctgtgcagcctctggaggcatcattagttcctcttggatgagctgggtccgccaggctccagggaaggggctggaatgggtggccaacataaagcaagatggaagtgagaaatattatgtggactctgtgaagggccgattcaccatctccagagacaacgccaaagacttactgtatttgcaaatgaacagcctgagagtcgaggacacggctttatattactgtgcgagagatcgtccagtggctggtgcgtcggccctctggggccagggaaccctggtcaccgtctcctct

SEQ ID NO: 126 - DNA, 381 bp, Artificial Sequence; identical to SEQ ID NO: 120
  Synthetic: 4A3 VL DNA Sequence with signal sequence

SEQ ID NO: 127 - DNA, 414 bp, Artificial Sequence
  Synthetic: 9H9 VH DNA Sequence with signal sequence
atggaattggggctgagctgggttttccttgttgctattttagaaggtgtccagtgtgaggtgcaactggtggagtctgggggaggcttggtccagcctggggggtccctgagactctcctgtgcagcctctggaggcatcattagtacctattggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaggacacggctatgtattactgtgcgagagatcgtccagtggctggtgcgtcggccctctggggccagggaaccctggtcaccgtctcctca

SEQ ID NO: 128 - DNA, 381 bp, Artificial Sequence; identical to SEQ ID NO: 120
  Synthetic: 9H9 VL DNA Sequence with signal sequence

SEQ ID NO: 129 - DNA, 357 bp, Artificial Sequence
  Synthetic: 7H7 VH DNA Sequence without signal sequence
gaggtgcaactggtggagtctgggggaggcttggtccagcctggggggtccctgagactctcctgtgcagcctctggaggcaccattagtacctcttggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaggacacggctatatattactgtgcgagagatcgtccagtggctggtgcgtcggccctctggggccagggaaccctggtcaccgtctcctca

SEQ ID NO: 130 - DNA, 321 bp, Artificial Sequence
  Synthetic: 7H7 VL DNA Sequence without signal sequence
gacatccagatgacccagtctccttccaccctgtctgcatctgtaggagacagagtcaccatcacttgccgggccagtcagagtataagtggctggttggcctggtatcagcagaaacaagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtattatggttcttctcggacgttcggccaagggaccaatgtggaaatcaaa

SEQ ID NO: 131 - DNA, 357 bp, Artificial Sequence
  Synthetic: 1B3 VH DNA Sequence without signal sequence
gaggtgcaactggtggagtctgggggaggcttggtccagcctggggggtccctgagactctcctgtgcagcctctggaggcaccattagtacctcttggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaagacacggctatgtattactgtgcgagagatcgtccagtggctggtgcgtcggccctctggggccagggaaccctggtcaccgtctcctca

SEQ ID NO: 132 - DNA, 321 bp, Artificial Sequence
  Synthetic: 1B3 VL DNA Sequence without signal sequence
gacatccagatgacccagtctccttccaccctgtctgcatctgtaggagacagagtcaccatcacttgccgggccagtcagagtattagtggctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtattatggttcttctcggacgttcggccaagggaccaatgtggaaatcaaa

SEQ ID NO: 133 - DNA, 357 bp, Artificial Sequence
  Synthetic: 3B6 VH DNA Sequence without signal sequence
gaggtgcaactggtggagtctgggggaggcttggtccagcctggggggtccctgagactctcctgtgcagcctctggaggcacaaccagtacctattggatgagctgggtccgccaggctccagggaaggggctggaatgggtggccaacataaagcaagatggaagtgagaaatattatgtggactctgtgaagggccgattcaccatctccagagacaacgccaagaactcactgaatttgcaaatgaacagcctgagagtcgaggacacggctatatattactgtgcgagagatcgtccagtggctggtgcgtcggccctctggggccagggaaccctggtcaccgtctcctca

SEQ ID NO: 134 - DNA, 321 bp, Artificial Sequence; identical to SEQ ID NO: 132
  Synthetic: 3B6 VL DNA Sequence without signal sequence

SEQ ID NO: 135 - DNA, 357 bp, Artificial Sequence
  Synthetic: 8B1 VH DNA Sequence without signal sequence
gaggtgcgactggtggagtctgggggaggcttggtccagcctggggggtccctgagactctcctgtgcagcctctggagacataattagtacccattggatgagctgggtccgccaggctccagggaaggggctggaatgggtggccaacataaaacaagatggaagtgagaagtattatgtggactctgtgaagggccgattcaccatctccagagacaacgccaagaactcactgtatttgcaaatgaacaccctgagagtcgaggacacggctatatattactgtacgagagatcgtccagtggctggtgcgtcggccctctggggccagggaaccctggtcaccgtctcctca

SEQ ID NO: 136 - DNA, 321 bp, Artificial Sequence
  Synthetic: 8B1 VL DNA Sequence without signal sequence
gacatccagatgacccagtctccttccaccctgtctgcatctgtaggagacagagtcaccatcacttgccgggccagtcagagtattagtggctggttggcctggtatcagcagaaaccagggaaagcccctaagctcctgatctataaggcgtctagtttagaaagtggggtcccattaaggttcagcggcagtggatctgggacagaattcactctcaccatcagcagcctgcagcctgatgattttgcaacttattactgccaacagtattatggttcttctcggacgttcggccaagggaccaatgtggaaatcaaa

SEQ ID NO: 137 - DNA, 357 bp, Artificial Sequence
  Synthetic: 4A3 VH DNA Sequence without signal sequence
gaggtgcaactggtggagtctgggggaggcttggtccagccgggggggtccctgagactctcctgtgcagcctctggaggcatcattagttcctcttggatgagctgggtccgccaggctccagggaaggggctggaatgggtggccaacataaagcaagatggaagtgagaaatattatgtggactctgtgaagggccgattcaccatctccagagacaacgccaaagacttactgtatttgcaaatgaacagcctgagagtcgaggacacggctttatattactgtgcgagagatcgtccagtggctggtgcgtcggccctctggggccagggaaccctggtcaccgtctcctct

SEQ ID NO: 138 - DNA, 321 bp, Artificial Sequence; identical to SEQ ID NO: 132
  Synthetic: 4A3 VL DNA Sequence without signal sequence

SEQ ID NO: 139 - DNA, 357 bp, Artificial Sequence
  Synthetic: 9H9 VH DNA Sequence without signal sequence
gaggtgcaactggtggagtctgggggaggcttggtccagcctggggggtccctgagactctcctgtgcagcctctggaggcatcattagtacctattggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaggacacggctatgtattactgtgcgagagatcgtccagtggctggtgcgtcggccctctggggccagggaaccctggtcaccgtctcctca

SEQ ID NO: 140 - DNA, 321 bp, Artificial Sequence; identical to SEQ ID NO: 132
  Synthetic: 9H9 VL DNA Sequence without signal sequence

SEQ ID NO: 141 - protein, 709 aa, Artificial Sequence
  Synthetic: 9H9-7A3 HL
EVQLVESGGGLVQPGGSLRLSCAASGGIISTYWMSWVRQAPGKGLEWVANIKQDGSEKYYVDSVKGRFTISRDNAKNSLYLQMNSLRVEDTAMYYCARDRPVAGASALWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGGGSQVQLVQSGAEVKKPGASVKVSCKASGYSFTGYTMHWVRQAPGQCLEWMGLINPYTGGTDYNQKFQGRVTMTVDKSTSTAYMELSSLRSEDTAVYYCARERPGGSQFIYYYPMDYWGQGTTVTVSSGGGGSGGGGSGGGGSGGGGSDIQMTQSPSSLSASVGDRVTITCRASANIYSYLAWYQQKPGKAPKFLVYNAITLAEGVPSRFSGSGSGTDFTLTISSLQPEDFATYYCQHHYGTPFTFGCGTKLEIK

SEQ ID NO: 142 - protein, 709 aa, Artificial Sequence
  Synthetic: 9H9-7A3 LH
EVQLVESGGGLVQPGGSLRLSCAASGGIISTYWMSWVRQAPGKGLEWVANIKQDGSEKYYVDSVKGRFTISRDNAKNSLYLQMNSLRVEDTAMYYCARDRPVAGASALWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGGGSDIQMTQSPSSLSASVGDRVTITCRASANIYSYLAWYQQKPGKAPKFLVYNAITLAEGVPSRFSGSGSGTDFTLTISSLQPEDFATYYCQHHYGTPFTFGCGTKLEIKGGGGSGGGGSGGGGSGGGGSQVQLVQSGAEVKKPGASVKVSCKASGYSFTGYTMHWVRQAPGQCLEWMGLINPYTGGTDYNQKFQGRVTMTVDKSTSTAYMELSSLRSEDTAVYYCARERPGGSQFIYYYPMDYWGQGTTVTVSS

SEQ ID NO: 143 - protein, 709 aa, Artificial Sequence
  Synthetic: 9H9-7B1 HL
EVQLVESGGGLVQPGGSLRLSCAASGGIISTYWMSWVRQAPGKGLEWVANIKQDGSEKYYVDSVKGRFTISRDNAKNSLYLQMNSLRVEDTAMYYCARDRPVAGASALWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGGGSQVQLVQSGAEVKKPGASVKVSCKASGYSFTGYTMHWVRQAPGQCLEWMGLINPYTGGTDYNQKFQGRVTMTVDRSTSTAYMELSSLRSEDTAVYYCARERPGGSQFIYYYALDYWGQGTTVTVSSGGGGSGGGGSGGGGSGGGGSDIQMTQSPSSLSASVGDRVTITCRASENIYSYLAWYQQKPGKAPKFLVYNADTLAEGVPSRFSGSGSGTDFTLTISSLQPEDFATYYCQHHYGTPFTFGCGTKLEIK

SEQ ID NO: 144 - protein, 709 aa, Artificial Sequence
  Synthetic: 9H9-7B1 LH
EVQLVESGGGLVQPGGSLRLSCAASGGIISTYWMSWVRQAPGKGLEWVANIKQDGSEKYYVDSVKGRFTISRDNAKNSLYLQMNSLRVEDTAMYYCARDRPVAGASALWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLMISRTPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSSGGGGSDIQMTQSPSSLSASVGDRVTITCRASENIYSYLAWYQQKPGKAPKFLVYNADTLAEGVPSRFSGSGSGTDFTLTISSLQPEDFATYYCQHHYGTPFTFGCGTKLEIKGGGGSGGGGSGGGGSGGGGSQVQLVQSGAEVKKPGASVKVSCKASGYSFTGYTMHWVRQAPGQCLEWMGLINPYTGGTDYNQKFQGRVTMTVDRSTSTAYMELSSLRSEDTAVYYCARERPGGSQFIYYYALDYWGQGTTVTVSS

SEQ ID NO: 145 - protein, 214 aa, Artificial Sequence
  Synthetic: 9H9 (light chain)
DIQMTQSPSTLSASVGDRVTITCRASQSISGWLAWYQQKPGKAPKLLIYKASSLESGVPSRFSGSGSGTEFTLTISSLQPDDFATYYCQQYYGSSRTFGQGTNVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - DNA, 2127 bp, Artificial Sequence
  Synthetic: 9H9 AQQ-7A3 H2L1
gaggtgcaactggtggagtctgggggaggcttggtccagcctggggggtccctgagactctcctgtgcagcctctggaggcatcattagtacctattggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaggacacggctatgtattactgtgcgagagatcgtccagtggctggtgcgtcggccctctggggccagggaaccctggtcaccgtctcctcagctagcacaaagggcccttccgtgtttccactggctcccagctctaagtccaccagcggaggaacagccgctctgggctgtctggtgaaggactatttcccagagcccgtgaccgtgagctggaactctggcgccctgaccagcggagtgcatacatttcctgctgtgctgcagtccagcggcctgtactctctgtcttccgtggtgaccgtgccaagctcttccctgggcacccagacatatatctgcaacgtgaatcacaagccatccaatacaaaggtggacaagaaggtggagcccaagagctgtgataagacccatacatgccccccttgtcctgctccagaggctcagggaggaccatccgtgttcctgtttccacccaagcctaaggacaccctgatgatctctaggaccccagaggtgacctgcgtggtggtggacgtgagccacgaggatcccgaggtgaagttcaactggtacgtggatggcgtggaggtgcataatgccaagacaaagccaagggaggagcagtacaatagcacctatcgggtggtgtctgtgctgacagtgctgcaccaggactggctgaacggcaaggagtacaagtgccaggtgtctaataaggccctgcccgctcctatcgagaagaccatctccaaggccaagggccagcctagggagccacaggtgtacacactgcctccaagccgggacgagctgaccaagaaccaggtgtctctgacatgtctggtgaagggcttctatccctctgatatcgctgtggagtgggagtccaatggccagcctgagaacaattacaagaccacaccccctgtgctggactccgatggcagcttctttctgtattccaagctgaccgtggataagagcaggtggcagcagggcaacgtgttttcttgttccgtgatgcatgaggctctgcacaatcattacacacagaagagcctgtctctgtcccctggcaaaggctcgagcgggggaggaggtagccaagtgcagctggtgcagagcggcgctgaggtgaagaagcccggcgccagcgtcaaggtcagctgtaaggccagcggctacagcttcactggctacactatgcactgggtgagacaagcccccggccaatgtctggagtggatgggactgatcaacccttacactggcggcactgactacaaccagaaattccaaggaagggtcacaatgactgtggacaagagcacatccactgcctacatggagctgagctctctgaggagcgaggacacagccgtgtactactgtgccagagagagacccggcggcagccagttcatctactactaccctatggactactggggccaaggcacaacagtgactgtgagcagcggagggggcggttccggaggaggcggcagcgggggaggaggtagcggcggaggtgggtctgacatccagatgacacagagcccaagctctctgagcgccagcgtgggagatagggtgacaatcacatgtagggccagcgccaacatctacagctatctggcttggtaccagcagaagcccggcaaggcccctaagtttctggtgtacaacgccatcactctggctgagggcgtgcctagcagatttagcggcagcggaagcggcacagacttcactctgacaatcagctctctgcagccagaggatttcgccacatactactgccagcaccactacggcactcctttcacattcggctgcggcactaagctggagatcaag

SEQ ID NO: 147 - DNA, 2127 bp, Artificial Sequence
  Synthetic: 9H9 AQQ-7A3 L1H2
gaggtgcaactggtggagtctgggggaggcttggtccagcctggggggtccctgagactctcctgtgcagcctctggaggcatcattagtacctattggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaggacacggctatgtattactgtgcgagagatcgtccagtggctggtgcgtcggccctctggggccagggaaccctggtcaccgtctcctcagctagcacaaagggcccttccgtgtttccactggctcccagctctaagtccaccagcggaggaacagccgctctgggctgtctggtgaaggactatttcccagagcccgtgaccgtgagctggaactctggcgccctgaccagcggagtgcatacatttcctgctgtgctgcagtccagcggcctgtactctctgtcttccgtggtgaccgtgccaagctcttccctgggcacccagacatatatctgcaacgtgaatcacaagccatccaatacaaaggtggacaagaaggtggagcccaagagctgtgataagacccatacatgccccccttgtcctgctccagaggctcagggaggaccatccgtgttcctgtttccacccaagcctaaggacaccctgatgatctctaggaccccagaggtgacctgcgtggtggtggacgtgagccacgaggatcccgaggtgaagttcaactggtacgtggatggcgtggaggtgcataatgccaagacaaagccaagggaggagcagtacaatagcacctatcgggtggtgtctgtgctgacagtgctgcaccaggactggctgaacggcaaggagtacaagtgccaggtgtctaataaggccctgcccgctcctatcgagaagaccatctccaaggccaagggccagcctagggagccacaggtgtacacactgcctccaagccgggacgagctgaccaagaaccaggtgtctctgacatgtctggtgaagggcttctatccctctgatatcgctgtggagtgggagtccaatggccagcctgagaacaattacaagaccacaccccctgtgctggactccgatggcagcttctttctgtattccaagctgaccgtggataagagcaggtggcagcagggcaacgtgttttcttgttccgtgatgcatgaggctctgcacaatcattacacacagaagagcctgtctctgtcccctggcaaaggctcgagcgggggaggaggtagcgacatccagatgacacagagcccaagctctctgagcgccagcgtgggagatagggtgacaatcacatgtagggccagcgccaacatctacagctatctggcttggtaccagcagaagcccggcaaggcccctaagtttctggtgtacaacgccatcactctggctgagggcgtgcctagcagatttagcggcagcggaagcggcacagacttcactctgacaatcagctctctgcagccagaggatttcgccacatactactgccagcaccactacggcactcctttcacattcggctgcggcactaagctggagatcaagggagggggcggttccggaggaggcggcagcgggggaggaggtagcggcggaggtgggtctcaagtgcagctggtgcagagcggcgctgaggtgaagaagcccggcgccagcgtcaaggtcagctgtaaggccagcggctacagcttcactggctacactatgcactgggtgagacaagcccccggccaatgtctggagtggatgggactgatcaacccttacactggcggcactgactacaaccagaaattccaaggaagggtcacaatgactgtggacaagagcacatccactgcctacatggagctgagctctctgaggagcgaggacacagccgtgtactactgtgccagagagagacccggcggcagccagttcatctactactaccctatggactactggggccaaggcacaacagtgactgtgagcagc

SEQ ID NO: 148 - DNA, 2127 bp, Artificial Sequence
  Synthetic: 9H9 AQQ-7B1 H2L1
gaggtgcaactggtggagtctgggggaggcttggtccagcctggggggtccctgagactctcctgtgcagcctctggaggcatcattagtacctattggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaggacacggctatgtattactgtgcgagagatcgtccagtggctggtgcgtcggccctctggggccagggaaccctggtcaccgtctcctcagctagcacaaagggcccttccgtgtttccactggctcccagctctaagtccaccagcggaggaacagccgctctgggctgtctggtgaaggactatttcccagagcccgtgaccgtgagctggaactctggcgccctgaccagcggagtgcatacatttcctgctgtgctgcagtccagcggcctgtactctctgtcttccgtggtgaccgtgccaagctcttccctgggcacccagacatatatctgcaacgtgaatcacaagccatccaatacaaaggtggacaagaaggtggagcccaagagctgtgataagacccatacatgccccccttgtcctgctccagaggctcagggaggaccatccgtgttcctgtttccacccaagcctaaggacaccctgatgatctctaggaccccagaggtgacctgcgtggtggtggacgtgagccacgaggatcccgaggtgaagttcaactggtacgtggatggcgtggaggtgcataatgccaagacaaagccaagggaggagcagtacaatagcacctatcgggtggtgtctgtgctgacagtgctgcaccaggactggctgaacggcaaggagtacaagtgccaggtgtctaataaggccctgcccgctcctatcgagaagaccatctccaaggccaagggccagcctagggagccacaggtgtacacactgcctccaagccgggacgagctgaccaagaaccaggtgtctctgacatgtctggtgaagggcttctatccctctgatatcgctgtggagtgggagtccaatggccagcctgagaacaattacaagaccacaccccctgtgctggactccgatggcagcttctttctgtattccaagctgaccgtggataagagcaggtggcagcagggcaacgtgttttcttgttccgtgatgcatgaggctctgcacaatcattacacacagaagagcctgtctctgtcccctggcaaaggctcgagcgggggaggaggtagccaagtgcagctggtgcagagcggcgctgaggtgaagaagcccggcgccagcgtgaaagtgagctgtaaggcctccggctacagcttcactggctacactatgcactgggtcagacaagcccccggccaatgcctggagtggatgggactgatcaacccttacactggcggcactgactacaaccagaagttccaaggaagggtgactatgactgtggataggtccacaagcacagcctacatggagctgtcctctctgagatccgaggacactgccgtgtactactgtgctagggagagacccggcggcagccagttcatctactactacgctctggactactggggccaaggcacaacagtcacagtgagcagcggagggggcggttccggaggaggcggcagcgggggaggaggtagcggcggaggtgggtctgacatccagatgacacagagcccaagctctctgagcgccagcgtgggagatagggtgacaatcacttgtagggccagcgagaacatctacagctatctggcttggtaccagcagaagcccggcaaggccccaaagtttctggtgtacaacgccgacactctcgctgagggcgtcccttctaggttttccggcagcggctccggcactgacttcacactgactatcagctctctgcagccagaggatttcgccacatactactgccagcaccactacggcactcctttcacattcggctgcggcactaagctggagatcaag

SEQ ID NO: 149 - DNA, 2127 bp, Artificial Sequence
  Synthetic: 9H9 AQQ-7B1 L1H2
gaggtgcaactggtggagtctgggggaggcttggtccagcctggggggtccctgagactctcctgtgcagcctctggaggcatcattagtacctattggatgagctgggtccgccaggctccagggaaggggctggaatgggtggccaacataaagcaagatggaagtgagaaatattatgtggactctgtgaagggccgattcaccatctccagagacaacgccaagaactcactgtatttgcaaatgaacagcctgagagtcgaggacacggctatgtattactgtgcgagagatcgtccagtggctggtgcgtcggccctctggggccagggaaccctggtcaccgtctcctcagctagcacaaagggcccttccgtgtttccactggctcccagctctaagtccaccagcggaggaacagccgctctgggctgtctggtgaaggactatttcccagagcccgtgaccgtgagctggaactctggcgccctgaccagcggagtgcatacatttcctgctgtgctgcagtccagcggcctgtactctctgtcttccgtggtgaccgtgccaagctcttccctgggcacccagacatatatctgcaacgtgaatcacaagccatccaatacaaaggtggacaagaaggtggagcccaagagctgtgataagacccatacatgccccccttgtcctgctccagaggctcagggaggaccatccgtgttcctgtttccacccaagcctaaggacaccctgatgatctctaggaccccagaggtgacctgcgtggtggtggacgtgagccacgaggatcccgaggtgaagttcaactggtacgtggatggcgtggaggtgcataatgccaagacaaagccaagggaggagcagtacaatagcacctatcgggtggtgtctgtgctgacagtgctgcaccaggactggctgaacggcaaggagtacaagtgccaggtgtctaataaggccctgcccgctcctatcgagaagaccatctccaaggccaagggccagcctagggagccacaggtgtacacactgcctccaagccgggacgagctgaccaagaaccaggtgtctctgacatgtctggtgaagggcttctatccctctgatatcgctgtggagtgggagtccaatggccagcctgagaacaattacaagaccacaccccctgtgctggactccgatggcagcttctttctgtattccaagctgaccgtggataagagcaggtggcagcagggcaacgtgttttcttgttccgtgatgcatgaggctctgcacaatcattacacacagaagagcctgtctctgtcccctggcaaaggctcgagcgggggaggaggtagcgacatccagatgacacagagcccaagctctctgagcgccagcgtgggagatagggtgacaatcacttgtagggccagcgagaacatctacagctatctggcttggtaccagcagaagcccggcaaggccccaaagtttctggtgtacaacgccgacactctcgctgagggcgtcccttctaggttttccggcagcggctccggcactgacttcacactgactatcagctctctgcagccagaggatttcgccacatactactgccagcaccactacggcactcctttcacattcggctgcggcactaagctggagatcaagggagggggcggttccggaggaggcggcagcgggggaggaggtagcggcggaggtgggtctcaagtgcagctggtgcagagcggcgctgaggtgaagaagcccggcgccagcgtgaaagtgagctgtaaggcctccggctacagcttcactggctacactatgcactgggtcagacaagcccccggccaatgcctggagtggatgggactgatcaacccttacactggcggcactgactacaaccagaagttccaaggaagggtgactatgactgtggataggtccacaagcacagcctacatggagctgtcctctctgagatccgaggacactgccgtgtactactgtgctagggagagacccggcggcagccagttcatctactactacgctctggactactggggccaaggcacaacagtcacagtgagcagc

SEQ ID NO: 150 - DNA, 642 bp, Artificial Sequence
  Synthetic: 9H9 LC
gacatccagatgacccagtctccttccaccctgtctgcatctgtaggagacagagtcaccatcacttgccgggccagtcagagtattagtggctggttggcctggtatcagcagaaaccagggaaagcccctaagctcctgatctataaggcgtctagtttagaaagtggggtcccatcaaggttcagcggcagtggatctgggacagagttcactctcaccatcagcagcctgcagcctgatgattttgcaacttattactgccaacagtattatggttcttctcggacgttcggccaagggaccaatgtggaaatcaaacgtacggtggccgctccaagcgtgttcatctttcccccttctgacgagcagctgaagtctggcacagcctccgtggtgtgcctgctgaacaacttctaccccagagaggccaaggtgcagtggaaggtggataacgctctgcagtctggcaattcccaggagagcgtgaccgagcaggactctaaggattccacatatagcctgagctctaccctgacactgtctaaggccgattacgagaagcacaaggtgtatgcttgcgaggtgacccatcagggcctgtccagcccagtgacaaagtccttcaatcgcggcgagtgt

(19) United States
(12) Patent Application Publication
(10) Pub. No.: US 2024/0417459 A1
(43) Pub. Date: Dec. 19, 2024

(54) ANTIBODIES AGAINST ILT4, BISPECIFIC ANTI-ILT4/PD-L1 ANTIBODY AND USES THEREOF

(71) Applicant: Celldex Therapeutics, Inc., Hampton, NJ (US)

(72) Inventors: Tibor KELER, Pipersville, PA (US); Joel GOLDSTEIN, Hopewell, NJ (US); Michael B. MURPHY, Wallingford, CT (US); Thomas O'NEILL, Washington, NJ (US); Laura A. VITALE, Doylestown, PA (US)

(21) Appl. No.: 18/283,742

(22) PCT Filed: Apr. 8, 2022
(86) PCT No.: PCT/US2022/023961

§ 371 Date: Sep. 22, 2023

Related U.S. Application Data
(60) Provisional application No. 63/172,997, filed on Apr. 9, 2021.

(51) Int. Cl. C07K 16/28

(52) U.S. Cl. CPC C07K 16/2803; C07K 16/2818; C07K 16/2827; C07K 2317/24; C07K 2317/31; C07K 2317/622

(57) ABSTRACT

Provided herein are novel ILT4 antibodies, and antigen binding fragments thereof, as well as bispecific and multispecific constructs binding to ILT4 and PD-L1, comprising such antibodies linked to at least one additional binding agent. Methods of inducing or enhancing an immune response, and methods of treating cancer, by administering the antibodies (or fragments), bispecific constructs, or compositions also are described.

This application claims the benefit of U.S. Provisional Patent Application No. 63/172,997, filed Apr. 9, 2021, the disclosure of which is incorporated by reference herein in its entirety.

### I. BACKGROUND OF THE INVENTION

The inhibitory immune checkpoint receptor “immunoglobulin-like transcript 4” (ILT4) is a member of the non-catalytic tyrosine-phosphorylated receptor family which is expressed on immune cells (such as T cells, B cells, NK cells, dendritic cells, macrophages and mast cells). Like other receptors in this family, ILT4 contains a conserved sequence of amino acids in the cytoplasmic domain referred to as an immunoreceptor tyrosine-based inhibitory motif (ITIM). (Veillette et al. (2002) Annual Review of Immunology 20(1):669-707). Binding and activation of ILT4 by its cognate ligands (HLA-G and HLA Class I in myeloid cells) has immunosuppressive effects through multiple mechanisms. ILT4 is also found in tumor cells and stroma cells in the tumor microenvironment of various cancers and has been shown to modulate the biological behaviors of tumor cells, thus promoting their immune escape. (Gao et al. (2018) Biochimica et Biophysica Acta (BBA)—Reviews on Cancer 1869(2):278-285). Accordingly, the expression of ILT4 in several tumor types is associated with poor outcome.

Programmed death-ligand 1 (PD-L1) is a 40 kDa type 1 transmembrane protein associated with suppressing the immune system during particular events such as pregnancy, tissue allografts, autoimmune disease, and other disease states such as hepatitis. Normally the immune system reacts to foreign antigens that are associated with exogenous or endogenous danger signals, which triggers a proliferation of antigen-specific CD8+ T cells and/or CD4+ helper cells. Binding of PD-L1 to the receptor, Programmed cell death protein 1 (PD-1), transmits an inhibitory signal that reduces the proliferation of these T cells and can also induce apoptosis, which is further mediated by a lower regulation of the gene Bcl-2. In addition to the well-established inhibitory role of PD-1 on T cells, its expression is also observed on tumor infiltrating macrophages where it can negatively regulate anti-tumor functions such as phagocytosis (Gordon et al. (2017) Nature 545: 495-9). PD-L1 is abundant in a variety of human cancers (Dong et al. (2002) Nat. Med. 8:787-9). The interaction between PD-1 and PD-L1 results in a decrease in tumor infiltrating lymphocytes, a decrease in T-cell receptor mediated proliferation, and immune evasion by the cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1, and the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66).

Despite advances associated with antibody therapy, there is a need in the art for new and improved therapeutic agents to treat conditions or diseases, e.g., in which stimulation of an immune response is desired. Accordingly, it is an object of the present invention to provide improved methods for treating subjects with such conditions or diseases, such as cancer.

### II. SUMMARY OF THE INVENTION

Provided herein are novel antibodies which bind to human ILT4, and antigen binding fragments thereof (e.g., fragments such as an Fab, Fab′, F(ab′)₂, Fv, or a single chain Fv). Bispecific and multispecific constructs comprising such antibodies (or fragments) linked to at least one additional binding agent (e.g., a ligand, receptor/trap sequences, or an antibody or antigen binding fragment thereof) also are described, e.g., an additional antibody (or fragment) which binds to human PD-L1 and/or human PD-1. As further described herein, the ILT4 antibodies (and fragments) and constructs of the present invention can be used in methods of inducing or enhancing an immune response and methods of treating a disease or condition (e.g., cancer).

In one embodiment, the ILT4 antibody or antigen binding fragment thereof comprises heavy and light chain CDR1, CDR2 and CDR3 domains having the following sequences:
(i) a heavy chain variable region CDR1 amino acid sequence selected from the consensus sequence: G Y T (I,M) H (SEQ ID NO: 21), or conservative sequence modifications thereof;
(ii) a heavy chain variable region CDR2 amino acid sequence as set forth in SEQ ID NO:3, or conservative sequence modifications thereof;
(iii) a heavy chain variable region CDR3 amino acid sequence selected from the consensus sequence: E R P G G S Q F I Y Y Y (P,A) (M,L) D Y (SEQ ID NO:22), or conservative sequence modifications thereof;
(iv) a light chain variable region CDR1 amino acid sequence selected from the consensus sequence: R A S (A,E) N I Y S Y L A (SEQ ID NO: 23), or conservative sequence modifications thereof;
(v) a light chain variable region CDR2 amino acid sequence selected from the consensus sequence: N A (I,D) T L A E (SEQ ID NO: 24), or conservative sequence modifications thereof;
(vi) a light chain variable region CDR3 amino acid sequence as set forth in SEQ ID NO:8, or conservative sequence modifications thereof.

An exemplary ILT4 antibody is antibody 7A3 as described herein. In one embodiment, the ILT4 antibody or antigen binding fragment thereof comprises the heavy and light chain CDRs or variable regions of antibody 7A3. In another embodiment, the antibody or antigen binding fragment thereof comprises the CDR1, CDR2, and CDR3 domains of the heavy chain variable region of antibody 7A3 having the sequence set forth in SEQ ID NO:9, and the CDR1, CDR2 and CDR3 domains of the light chain variable region of antibody 7A3 having the sequence set forth in SEQ ID NO:10. In another embodiment, the antibody or antigen binding thereof comprises heavy chain CDR1, CDR2 and CDR3 domains having the sequences set forth in SEQ ID NOs:1, 3, and 5, respectively, or conservative sequence modifications thereof, and light chain CDR1, CDR2 and CDR3 domains having the sequences set forth in SEQ ID NOs:6, 7 and 8, respectively, or conservative sequence modifications thereof. In another embodiment, the antibody or antigen binding thereof comprises a heavy chain variable region having the amino acid sequence set forth in SEQ ID NO:9. In another embodiment, the antibody or antigen binding thereof comprises a light chain variable region having the amino acid sequence set forth in SEQ ID NO:10. In another embodiment, the antibody or antigen binding fragment thereof comprises heavy and light chain variable regions having the amino acid sequences set forth in SEQ ID NO:9 and SEQ ID NO:10, respectively. In another embodiment, the antibody or antigen binding thereof comprises a heavy chain having the amino acid sequence set forth in SEQ ID NO:25. In another embodiment, the antibody or antigen binding thereof comprises a light chain having the amino acid sequence set forth in SEQ ID NO:26. In another embodiment, the antibody comprises heavy and light chains having the amino acid sequences set forth in SEQ ID NO:25 and SEQ ID NO:26, respectively.

Another exemplary ILT4 antibody is antibody 7B1 as described herein. In one embodiment, the ILT4 antibody or antigen binding fragment thereof comprises the heavy and light chain CDRs or variable regions of antibody 7B1. In another embodiment, the antibody or antigen binding fragment thereof comprises the CDR1, CDR2, and CDR3 domains of the heavy chain variable region of antibody 7B1 having the sequence set forth in SEQ ID NO:19, and the CDR1, CDR2 and CDR3 domains of the light chain variable region of antibody 7B1 having the sequence set forth in SEQ ID NO:20. In another embodiment, the antibody or antigen binding fragment thereof comprises heavy chain CDR1, CDR2 and CDR3 domains having the sequences set forth in SEQ ID NOs:11, 13, and 15, respectively, or conservative sequence modifications thereof, and light chain CDR1, CDR2 and CDR3 domains having the sequences set forth in SEQ ID NOs: 16, 17, and 18, respectively, or conservative sequence modifications thereof. In another embodiment, the antibody or antigen binding fragment thereof comprises a heavy chain variable region having the amino acid sequences set forth in SEQ ID NO:19. In another embodiment, the antibody or antigen binding fragment thereof comprises a light chain variable region having the amino acid sequences set forth in SEQ ID NO:20. In another embodiment, the antibody or antigen binding fragment thereof comprises heavy and light chain variable regions having the amino acid sequences set forth in SEQ ID NO:19 and SEQ ID NO:20, respectively. In another embodiment, the antibody or antigen binding thereof comprises a heavy chain having the amino acid sequence set forth in SEQ ID NO:27. In another embodiment, the antibody or antigen binding thereof comprises a light chain having the amino acid sequence set forth in SEQ ID NO:28. In another embodiment, the antibody comprises heavy and light chains having the amino acid sequences set forth in SEQ ID NO:27 and SEQ ID NO:28, respectively.

In yet other embodiments, the ILT4 antibodies (or antigen binding fragments thereof) of the present invention comprise:
(a) a heavy chain variable region amino acid sequence selected from the group consisting of SEQ ID NO:9, 19, 97, 98, 99, 103, 104, and 105, or a sequence at least 80% identical to any one of the aforementioned sequences; and/or
(b) a light chain variable region amino acid sequence selected from the group consisting of SEQ ID NO:10, 20, 100, 101, 102, 106, 107, and 108, or a sequence at least 80% identical to any one of the aforementioned sequences.

In another embodiment, the ILT4 antibodies of the present invention comprise:
(a) a heavy chain amino acid sequence as set forth in SEQ ID NO:25, 27, or a sequence at least 80% identical to either sequence; and/or
(b) a light chain amino acid sequence as set forth in SEQ ID NO:26, 28, or a sequence at least 80% identical to either sequence.

In another embodiment, the sequences of the ILT4 antibodies or antigen binding fragments thereof (e.g., CDR and/or variable region sequences) can comprise the exact amino acid sequences as the antibodies described herein (e.g., antibodies 7A3 or 7B1). In another embodiment, the antibodies comprise sequences of antibodies 7A3 or 7B1 which include conservative sequence modification, yet still retain the ability of to bind ILT4 effectively. Such sequence modifications may include one or more (e.g., 1, 2, 3, 4, 5, or 6) amino acid additions, deletions, or substitutions, e.g., conservative sequence modifications.

In another embodiment, the antibodies comprise sequences which share at least 80% sequence identity to the sequences of antibodies 7A3 or 7B1. Sequences substantially identical to the ILT4 antibodies or antigen binding fragments described herein (e.g., at least 80%, 85%, 90%, 91%, 92%, 93%, 94%, 95%, 96%, 97%, 98% or 99% identical to the aforementioned sequences), are also encompassed by the invention. In one embodiment, the ILT4 antibody or antigen binding fragment thereof comprises a heavy chain variable region comprising SEQ ID NO:9, SEQ ID NO: 19, or a sequence at least 90% identical thereto (e.g., at least 90%, 91%, 92%, 93%, 94%, 95%, 96%, 97%, 98% or 99% identical to the aforementioned sequences). In another embodiment, the ILT4 antibody or antigen binding fragment thereof comprises a light chain variable region comprising SEQ ID NO:10, SEQ ID NO:20, or a sequence at least 90% identical thereto (e.g., at least 90%, 91%, 92%, 93%, 94%, 95%, 96%, 97%, 98% or 99% identical to the aforementioned sequences). In another embodiment, the ILT4 antibody or antigen binding fragment thereof comprises a heavy chain variable region comprising SEQ ID NO:9 and a light chain variable region comprising SEQ ID NO:10 or sequences at least 90% identical thereto (e.g., at least 90%, 91%, 92%, 93%, 94%, 95%, 96%, 97%, 98% or 99% identical to the aforementioned sequences). In another embodiment, the ILT4 antibody or antigen binding fragment thereof comprises a heavy chain variable region comprising SEQ ID NO:19 and a light chain variable region comprising SEQ ID NO:20 or sequences at least 90% identical thereto (e.g., at least 90%, 91%, 92%, 93%, 94%, 95%, 96%, 97%, 98% or 99% identical to the aforementioned sequences).

In another embodiment, the ILT4 antibody or antigen binding fragment thereof comprises a heavy chain comprising SEQ ID NO:25, SEQ ID NO: 27, or a sequence at least 90% identical thereto (e.g., at least 90%, 91%, 92%, 93%, 94%, 95%, 96%, 97%, 98% or 99% identical to the aforementioned sequences). In another embodiment, the ILT4 antibody or antigen binding fragment thereof comprises a light chain comprising SEQ ID NO:26, SEQ ID NO:28, or a sequence at least 90% identical thereto (e.g., at least 90%, 91%, 92%, 93%, 94%, 95%, 96%, 97%, 98% or 99% identical to the aforementioned sequences). In another embodiment, the ILT4 antibody comprises a heavy chain comprising SEQ ID NO:25 and a light chain comprising SEQ ID NO:26 or sequences at least 90% identical thereto (e.g., at least 90%, 91%, 92%, 93%, 94%, 95%, 96%, 97%, 98% or 99% identical to the aforementioned sequences). In another embodiment, the ILT4 antibody comprises a heavy chain comprising SEQ ID NO:27 and a light chain comprising SEQ ID NO:28 or sequences at least 90% identical thereto (e.g., at least 90%, 91%, 92%, 93%, 94%, 95%, 96%, 97%, 98% or 99% identical to the aforementioned sequences).

ILT4 antibodies or antigen binding fragments thereof that compete for binding with any of the antibodies (or fragments) described herein, or that bind the same epitope as any of the antibodies (or fragments) described herein, are also encompassed by the present invention. For example, in one embodiment, the ILT4 antibody or antigen binding fragment thereof competes for binding to ILT4 with antibody 7A3 and/or antibody 7B1.

In another embodiment, the ILT4 antibodies or antigen binding fragments exhibit one or more of the following properties:
a. blocking ILT4 ligand (e.g., HLA-G ligand) binding to human ILT4;
b. enhancing or increasing cytokine or chemokine release by human macrophages;
c. potentiating the activation effects of LPS and IFNγ on macrophages;
d. promoting M1 macrophage polarization;
e. binding to human ILT4 with an equilibrium dissociation constant Kd of 10⁻⁹ M or less, or alternatively, an equilibrium association constant Ka of 10⁺⁹ M⁻¹ or greater;
f. lack of cross-reactivity with other ILT family members;
g. cross-reactivity with cynomolgus ILT4; and or
h. inhibiting tumor cells that express ILT4.

The present invention also provides bispecific constructs (or multispecific constructs) comprising the ILT4 antibodies or antigen binding fragments thereof linked to at least one additional binding agent (e.g., a ligand or an antibody or antigen binding fragment thereof, e.g., a PD-L1 or PD-1 antibody or antigen binding fragment thereof). In one embodiment, the bispecific construct comprises a PD-L1 antibody or antigen binding fragment thereof which comprises heavy and light chain CDR1, CDR2, and CDR3 amino acid sequences selected from the group consisting of:
(a) heavy chain variable region CDR1, CDR2 and CDR3 amino acid sequences as set forth in SEQ ID NOs: 59, 60, and 61, respectively, and light chain variable region CDR1, CDR2 and CDR3 amino acid sequences as set forth in SEQ ID NOs:62, 63, and 64, respectively;
(b) heavy chain variable region CDR1, CDR2 and CDR3 amino acid sequences as set forth in SEQ ID NOs: 35, 36, and 37, respectively, and light chain variable region CDR1, CDR2 and CDR3 amino acid sequences as set forth in SEQ ID NOs:38, 39, and 40, respectively;
(c) heavy chain variable region CDR1, CDR2 and CDR3 amino acid sequences as set forth in SEQ ID NOs: 41, 42, and 43, respectively, and light chain variable region CDR1, CDR2 and CDR3 amino acid sequences as set forth in SEQ ID NOs:44, 45, and 46, respectively;
(d) heavy chain variable region CDR1, CDR2 and CDR3 amino acid sequences as set forth in SEQ ID NOs: 47, 48, and 49, respectively, and light chain variable region CDR1, CDR2 and CDR3 amino acid sequences as set forth in SEQ ID NOs:50, 51, and 52, respectively;
(e) heavy chain variable region CDR1, CDR2 and CDR3 amino acid sequences as set forth in SEQ ID NOs: 53, 54, and 55, respectively, and light chain variable region CDR1, CDR2 and CDR3 amino acid sequences as set forth in SEQ ID NOs:56, 57, and 58, respectively; and
(f) heavy chain variable region CDR1, CDR2 and CDR3 amino acid sequences as set forth in SEQ ID NOs: 29, 30, and 31, respectively, and light chain variable region CDR1, CDR2 and CDR3 amino acid sequences as set forth in SEQ ID NOs:32, 33, and 34, respectively.

For example, the bispecific construct can be a chemical conjugate, which can be made by chemical conjugation of the ILT4 antibody and the second binding agent, e.g., a ligand or an antibody or antigen binding fragment thereof (such as a PD-L1 antibody or antigen binding fragment thereof). In one embodiment, the PD-L1 antibody or antigen binding fragment thereof further comprises a human IgG1 constant domain. In another embodiment, the ILT4 antibody or antigen binding fragment thereof is linked to the C-terminus of the heavy chain of the PD-L1 antibody or antigen binding fragment thereof. In another embodiment, the ILT4 antigen binding fragment thereof is a scFv, e.g., a scFv further comprising disulfide stabilization modifications with Cys substitutions at VH44 and VL100.

In another embodiment, the ILT4 antibody or antigen binding fragment thereof further comprises a human IgG1 constant domain. In another embodiment, the PD-L1 antibody or antigen binding fragment thereof is linked to the C-terminus of the heavy chain of the ILT4 antibody or antigen binding fragment thereof. In another embodiment, the PD-L1 antigen binding fragment thereof is a scFv.

In a particular embodiment, the bispecific construct comprises a PD-L1 antibody linked to an ILT4 scFv, wherein:
(a) the PD-L1 antibody, or antigen binding fragment thereof, comprises heavy chain variable region CDR1, CDR2 and CDR3 amino acid sequences as set forth in SEQ ID NOs: 59, 60, and 61, respectively, and light chain variable region CDR1, CDR2 and CDR3 amino acid sequences as set forth in SEQ ID NOs:62, 63, and 64, respectively; and
(b) the ILT4 scFv comprises:
   (i) heavy chain variable region CDR1, CDR2 and CDR3 amino acid sequences as set forth in SEQ ID NOs:1, 3, and 5, respectively, and light chain variable region CDR1, CDR2 and CDR3 amino acid sequences as set forth in SEQ ID NOs:6, 7, and 8, respectively; or
   (ii) heavy chain variable region CDR1, CDR2 and CDR3 amino acid sequences as set forth in SEQ ID NOs:11, 13, and 15, respectively, and light chain variable region CDR1, CDR2 and CDR3 amino acid sequences as set forth in SEQ ID NOs:16, 17, and 18, respectively.

In another embodiment, the ILT4 scFv of the bispecific (or multispecific) construct further comprises disulfide stabilization modifications with Cys substitutions at VH44 and VL100.

The present invention also provides compositions comprising any of the bispecific constructs (multispecific constructs), antibodies, or antigen binding fragments thereof, described herein and a pharmaceutically acceptable carrier, as well as kits comprising any of the bispecific constructs (multispecific constructs), antibodies, or antigen binding fragments thereof, described herein and instructions for use.

In a further aspect, isolated nucleic acid molecules encoding the antibodies, or antigen binding fragments thereof, and bispecific or multispecific constructs described herein are also provided, as well as expression vectors comprising such nucleic acids and host cells comprising such expression vectors. In another embodiment, a nucleic acid molecule coding for any of the antibodies, or antigen binding fragments thereof, or bispecific constructs described herein is provided. In another embodiment, the nucleic acid molecule is in the form of an expression vector. In another embodiment, the nucleic acid molecule is in the form of an expression vector which expresses the antibody, or antigen binding fragment thereof, or bispecific construct when administered to a subject in vivo.

In one embodiment, the nucleic acid molecule comprises a nucleotide sequence encoding an antibody heavy and/or light chain variable region, wherein the antibody variable region comprises the amino acid sequence as set forth in SEQ ID NO:9, 10, 19, 20, 97, 98, 99, 100, 101, 102, 103, 104, 105, 106, 107, 108, or an amino acid sequence at least 90% identical thereto (e.g., at least 90%, 91%, 92%, 93%, 94%, 95%, 96%, 97%, 98% or 99% identical to one or more of the aforementioned sequences).

In another embodiment, the nucleic acid molecule comprises a nucleotide sequence encoding an antibody heavy and/or light chain, wherein the antibody chain comprises the amino acid sequence as set forth in SEQ ID NO:25, 26, 27, 28, or an amino acid sequence at least 90% identical thereto (e.g., at least 90%, 91%, 92%, 93%, 94%, 95%, 96%, 97%, 98% or 99% identical to one or more of the aforementioned sequences).

In another embodiment, the nucleic acid molecule comprises a nucleotide sequence encoding heavy and light chain variable regions of an antibody, wherein the heavy and light chain variable regions comprise the amino acid sequences as set forth in SEQ ID NOs:9 and 108 or SEQ ID NOs:19 and 20, respectively, or amino acids sequences at least 90% identical thereto (e.g., at least 90%, 91%, 92%, 93%, 94%, 95%, 96%, 97%, 98% or 99% identical the aforementioned sequences).

In another embodiment, the nucleic acid molecule comprises a nucleotide sequence encoding heavy and light chains of an antibody, wherein the heavy and light chains comprise the amino acid sequences as set forth in SEQ ID NOs:25 and 26 or SEQ ID NOs:27 and 28, respectively, or amino acids sequences at least 90% identical thereto (e.g., at least 90%, 91%, 92%, 93%, 94%, 95%, 96%, 97%, 98% or 99% identical the aforementioned sequences).

In another aspect, methods for inducing or enhancing an immune response (e.g., against an antigen) in a subject comprising administering to the subject any one of the antibodies, or antigen binding fragments thereof, bispecific constructs, multispecific constructs, or the compositions described herein, in an amount effective to induce or enhance an immune response in the subject (e.g., against an antigen).

In a further aspect, methods of for treating a condition or disease in a subject (e.g., cancer) are provided, the method comprising administering to the subject any one of the antibodies, or antigen binding fragments thereof, bispecific constructs, multispecific constructs, or the compositions described herein, in an amount effective to treat the condition or disease.

In another embodiment, methods for treating a tumor (e.g., a tumor expressing ILT4, HLA-G, HLA class I, angiopoietin like 2, Nogo, or an ILT4 ligand) in a subject are provided, the method comprising administering to the subject any one of the antibodies, or antigen binding fragments thereof, bispecific constructs, multispecific constructs, or the compositions described herein, in an amount effective to treat the tumor.

The subject can be, for example, one who suffers from a condition or disease in which stimulation of an immune response is desired. In one embodiment, the condition or disease in which stimulation of an immune response is desired is cancer. The method of inducing or enhancing an immune response (e.g., against an antigen) in a subject can further comprise administering the antigen to the subject. Preferred antigens to be co-administered with the antibodies, or antigen binding fragments thereof, bispecific constructs, multispecific constructs, or the compositions of described herein are tumor antigens.

### III. BRIEF DESCRIPTION OF THE DRAWINGS

FIGS. 1A and 1B are tables showing the antigen binding kinetics for antibody 7A3 and its constructs (FIG. 1A) and antibody 7B1 and its constructs (FIG. 1B).

FIGS. 2A, 2B, and 2C are graphs showing representative traces of the antigen binding kinetic data for antibody 7A3 (FIG. 2A), antibody 7B1 (FIG. 2B), and control antibody (FIG. 2C).

FIGS. 3A and 3B are graphs showing binding of chimeric and humanized monoclonal antibodies to human ILT4 using ELISA; antibody 7A3 (FIG. 3A) and antibody 7B1 (FIG. 3B).

FIGS. 4A and 4B are graphs showing binding to HEK293 cells expressing human ILT4 for antibody 7A3 and its constructs (FIG. 4A) and antibody 7B1 and its constructs (FIG. 4B).

FIGS. 5A and 5B are graphs showing macrophage TNF-α production for antibody 7A3 and its constructs (FIG. 5A) and antibody 7B1 and its constructs (FIG. 5B).

FIGS. 6A-6F are graphs showing the induction of TNF-α and MIP1-γ production by humanized antibodies 7A3 VH6-L17 and 7B1 VH10-L21 in macrophages; untreated (FIGS. 6A and 6D), with LPS (FIGS. 6B and E), and with IFN-γ (FIGS. 6C and 6F).

FIGS. 7A, 7B, and 7C are graphs showing relative gene expression in humanized antibodies 7A3 VH6-L17 and 7B1 VH10-L21; FIG. 7A (CD86), FIG. 7B (CD54), and FIG. 7C (iNOS).

FIGS. 8A (antibody 7A3) and 8B (antibody 7B1) are graphs showing cross-reactivity of humanized monoclonal antibodies 7A3 VH6-L17 and 7B1 VH10-L21 to cells expressing ILT family members.

FIGS. 9A (monocytes), 9B (macrophages), and 9C (dendritic cells) are graphs showing binding of humanized monoclonal antibodies 7A3 VH6-L17 and 7B1 VH10-L21 to myeloid cells.

FIGS. 10A (PD-L1×ILT4) and 10B (PD-1×ILT4) are schematics showing the depiction of the bispecific constructs.

FIG. 11 is a table showing the antigen binding kinetics to human ILT4 for the bispecific antibody constructs.

FIGS. 12A (9H9-7A3 HL and 9H9-7A3 LH) and 12B (9H9-7B1 HL and 9H9-7B1 LH) are graphs showing humanized bispecific antibody binding characteristics to human PD-L1 with ELISA.

FIGS. 13A (9H9-7A3 HL and 9H9-7A3 LH) and 13B (9H9-7B1 HL and 9H9-7B1 LH) are graphs showing humanized bispecific antibody binding characteristics to HEK293 cells expressing human PD-L1.

FIGS. 14A (9H9-7A3 HL and 9H9-7A3 LH) and 14B (9H9-7B1 HL and 9H9-7B1 LH) are graphs showing humanized bispecific antibody binding characteristics to HEK293 cells expressing human ILT4.

FIGS. 15A (9H9-7A3 HL and 9H9-7A3 LH) and 15B (9H9-7B1 HL and 9H9-7B1 LH) are graphs showing bifunctional binding characteristics of humanized antibodies to HEK293 cells expressing human ILT4 and PD-L1.

FIGS. 16A (9H9-7A3 HL and 9H9-7A3 LH) and 16B (9H9-7B1 HL and 9H9-7B1 LH) are graphs showing T cell PD-1/PD-L1 blockade by humanized bispecific antibodies.

FIGS. 17A (9H9-7A3 HL and 9H9-7A3 LH) and 17B (9H9-7B1 HL and 9H9-7B1 LH) are graphs showing induction of TNF-α production by humanized bispecific antibodies in macrophages.

FIGS. 18A (9H9-7A3 HL and 9H9-7A3 LH) and 18B (9H9-7B1 HL and 9H9-7B1 LH) are graphs showing inhibition of HLA-G binding to ILT4 by humanized bispecific antibodies.

### IV. DETAILED DESCRIPTION OF THE INVENTION

In order that the present invention may be more readily understood, certain terms are first defined. Additional definitions are set forth throughout the detailed description.

### A. Definitions

The term “immunoglobulin-like transcript 4” or “ILT4” as used herein refers to an inhibitory immune checkpoint receptor and a member of the non-catalytic tyrosine-phosphorylated receptor family. ILT4 is also referred to as leukocyte immunoglobulin like receptor B2 (LILRB2), LIR2, MIR10, and CD85d. ILT4 is expressed on immune cells where it binds to MHC class I molecules on antigen-presenting cells and transduces a negative signal that inhibits stimulation of an immune response, e.g., by controlling inflammatory responses and cytotoxicity to focus the immune response and limit autoreactivity. Multiple isoforms of human ILT4 have been identified. Isoform 1 (Accession No. Q8N423-1; SEQ ID NO: 89) represents the canonical sequence, consisting of 598 amino acid residues. ILT4 antibodies (or antigen binding fragments thereof) of the invention may cross-react with ILT4 from species other than human. Alternatively, the ILT4 antibodies or antigen binding fragments thereof may be specific for human ILT4 and may not exhibit any cross-reactivity with other species. ILT4 or any variants and isoforms thereof, may either be isolated from cells or tissues which naturally express them or be recombinantly produced using well-known techniques in the art and/or those described herein.

Ligands which bind ILT4 are known in the art and include, among others, HLA-G, HLA class I, angiopoietin like 2, b-amyloid, SEMA4A, CD1c/d, CSPs, and myelin inhibitors such as Nogo66, MAG, OMgp.

The terms “human leukocyte antigen G” or “HLA-G” (also known as “histocompatibility antigen, class I, G”), refers to a ligand for ILT4. HLA-G belongs to the HLA nonclassical class I heavy chain paralogues. This class I molecule is a heterodimer consisting of a heavy chain and a light chain (beta-2 microglobulin). The heavy chain is anchored in the membrane. HLA-G is expressed on fetal derived placental cells. The heavy chain is approximately 45 kDa and its gene contains 8 exons.

As used herein, the terms “Programmed cell death 1 ligand 1”, “PD-L1”, “PDCD1 ligand 1”, “Programmed death ligand 1”, “B7 homolog 1”, “B7-H1” and “ILT44” are used interchangeably, and include variants, isoforms, species homologs of human PD-L1, and analogs having at least one common epitope with PD-L1. The complete PD-L1 sequence can be found under GenBank Accession No. NP_001254635 as set forth in SEQ ID NO:176.

Programmed death-ligand 1 (PD-L1) is a 40 kDa type 1 transmembrane protein that has been speculated to play a major role in suppressing the immune system during particular events such as pregnancy, tissue allografts, autoimmune disease, and other disease states such as hepatitis. Normally the immune system reacts to foreign antigens that are associated with exogenous or endogenous danger signals, which triggers a proliferation of antigen-specific CD8+ T cells and/or CD4+ helper cells. The binding of PD-L1 to PD-1 transmits an inhibitory signal that reduces the proliferation of these T cells and can also induce apoptosis, which is further mediated by a lower regulation of the gene Bcl-2. As used herein, the terms “Programmed Death 1,” “Programmed Cell Death 1,” “Protein PD-1,” “PD-1,” PD1,” “PDCD1,” “hPD-1” and “hPD-I” are used interchangeably, and include variants, isoforms, species homologs of human PD-1, and analogs having at least one common epitope with PD-1. The complete PD-1 sequence can be found under GenBank Accession No. NP_005009 as set forth in SEQ ID NO:175.

PD-L1 is abundant in a variety of human cancers (Dong et al. (2002) Nat. Med. 8:787-9). The interaction between PD-1 and PD-L1 results in a decrease in tumor infiltrating lymphocytes, a decrease in T-cell receptor mediated proliferation, and immune evasion by the cancerous cells (Dong et al. (2003) J. Mol. Med. 81:281-7; Blank et al. (2005) Cancer Immunol. Immunother. 54:307-314; Konishi et al. (2004) Clin. Cancer Res. 10:5094-100). Immune suppression can be reversed by inhibiting the local interaction of PD-1 with PD-L1, and the effect is additive when the interaction of PD-1 with PD-L2 is blocked as well (Iwai et al. (2002) Proc. Nat'l. Acad. Sci. USA 99:12293-7; Brown et al. (2003) J. Immunol. 170:1257-66).

As used herein, the term “subject” includes any human or non-human animal. For example, the methods and compositions of the present invention can be used to treat a subject with an immune disorder. The term “non-human animal” includes all vertebrates, e.g., mammals and non-mammals, such as non-human primates, sheep, dog, cow, chickens, amphibians, reptiles, etc.

The term “antibody” as referred to herein refers to a glycoprotein comprising at least two heavy (H) chains and two light (L) chains inter-connected by disulfide bonds, or an antigen binding fragment thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen binding fragment” of an antibody (or simply “antibody fragment”), as used herein, refers to one or more fragments or portions of an antibody that retain the ability to specifically bind to an antigen (e.g., human ILT4). Such “fragments” are, for example between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen binding function of an antibody can be performed by fragments of a full-length antibody. Examples of binding fragments encompassed within the term “antigen binding fragment” of an antibody include (i) a Fab fragment, a monovalent fragment consisting of the V_{L}, V_{H}, CL and CH1 domains; (ii) a F(ab′)₂ fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) or (vii) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (sFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen binding fragment” of an antibody. These antibody fragments are obtained using conventional techniques known to those with skill in the art, and the fragments are screened for utility in the same manner as are intact antibodies. Antigen binding fragments can be produced by recombinant DNA techniques, or by enzymatic or chemical cleavage of intact immunoglobulins.

As used herein, the term “binding domain” refers to the portion of a protein or antibody which comprises the amino acid residues that interact with an antigen. Binding domains include, but are not limited to, antibodies (e.g., full length antibodies), as well as antigen binding fragments thereof. The binding domain confers on the binding agent its specificity and affinity for the antigen. The term also covers any protein having a binding domain which is homologous or largely homologous to an immunoglobulin-binding domain. Such proteins may be derived from natural sources, or partly or wholly synthetically produced.

The term “monoclonal antibody,” as used herein, refers to an antibody that displays a single binding specificity and affinity for a particular epitope. Accordingly, the term “human monoclonal antibody” refers to an antibody which displays a single binding specificity and which has variable and optional constant regions derived from human germline immunoglobulin sequences. In one embodiment, human monoclonal antibodies are produced by a hybridoma that includes a B cell obtained from a transgenic non-human animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see, e.g., Lonberg (2005) Nature Biotech. 23(9):1117-1125), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen may not have sequence identity with the original nucleic acid molecules, but instead will be substantially identical or similar (i.e., have at least 80% identity).

The term “human antibody” includes antibodies having variable and constant regions (if present) of human germline immunoglobulin sequences. Human antibodies of the invention can include amino acid residues not encoded by human germline immunoglobulin sequences (e.g., mutations introduced by random or site-specific mutagenesis in vitro or by somatic mutation in vivo) (see, Lonberg, N. et al. (1994) Nature 368(6474): 856-859); Lonberg, N. (1994) Handbook of Experimental Pharmacology 113:49-101; Lonberg, N. and Huszar, D. (1995) Intern. Rev. Immunol. Vol. 13: 65-93, and Harding, F. and Lonberg, N. (1995) Ann. N. Y. Acad. Sci 764:536-546). However, the term “human antibody” does not include antibodies in which CDR sequences derived from the germline of another mammalian species, such as a mouse, have been grafted onto human framework sequences (i.e., chimeric and humanized antibodies).

A “humanized” antibody refers to an antibody in which some, most, or all of the amino acids outside the CDR domains of a non-human antibody are replaced with corresponding amino acids derived from human immunoglobulins. In one embodiment of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

An “isolated antibody,” as used herein, is intended to refer to an antibody which is substantially free of other antibodies having different antigenic specificities (e.g., an isolated antibody that specifically binds to human ILT4 is substantially free of antibodies that specifically bind antigens other than human ILT4; an isolated antibody that specifically binds to human PD-L1 is substantially free of antibodies that specifically bind antigens other than human PD-L1). An isolated antibody that specifically binds to an epitope may, however, have cross-reactivity to the same antigen from different species. In addition, an isolated antibody is typically substantially free of other cellular material and/or chemicals.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody (i.e., epitope mapping) are well known in the art and include, for example, immunoblotting and immunoprecipitation assays, wherein overlapping or contiguous peptides from the antigen (e.g., ILT4 or PD-L1) are tested for reactivity with the given antibody (e.g., an ILT4 or PD-L1 antibody. Methods of determining spatial conformation of epitopes include techniques in the art and those described herein, for example, x-ray crystallography and 2-dimensional nuclear magnetic resonance (see, e.g., Epitope Mapping Protocols in Methods in Molecular Biology, Vol. 66, G. E. Morris, Ed. (1996)).

The term “antibody that binds the same epitope” as another antibody is intended to encompass antibodies that interact with, i.e., bind to, the same structural region on human ILT4 as a reference ILT4 antibody. The “same epitope” to which the antibodies bind may be a linear epitope or a conformational epitope formed by tertiary folding of the antigen.

The term “competing antibody” refers to an antibody that competes for binding to human ILT4 with a reference ILT4 antibody, i.e., competitively inhibits binding of the reference ILT4 antibody to ILT4. A “competing antibody” may bind the same epitope on ILT4 as the reference ILT4 antibody, may bind to an overlapping epitope or may sterically hinder the binding of the reference ILT4 antibody to ILT4.

Antibodies that recognize the same epitope or compete for binding can be identified using routine techniques. Such techniques include, for example, an immunoassay, which shows the ability of one antibody to block the binding of another antibody to a target antigen, i.e., a competitive binding assay. Competitive binding is determined in an assay in which the immunoglobulin under test inhibits specific binding of a reference antibody to a common antigen, such as ILT4. Numerous types of competitive binding assays are known, for example: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using I-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)). Typically, such an assay involves the use of purified antigen bound to a solid surface or cells bearing either of these, an unlabeled test immunoglobulin and a labeled reference immunoglobulin. Competitive inhibition is measured by determining the amount of label bound to the solid surface or cells in the presence of the test immunoglobulin. Usually the test immunoglobulin is present in excess. Usually, when a competing antibody is present in excess, it will inhibit specific binding of a reference antibody to a common antigen by at least 50-55%, 55-60%, 60-65%, 65-70% 70-75% or more.

Other techniques include, for example, epitope mapping methods, such as, x-ray analyses of crystals of antigen:antibody complexes which provides atomic resolution of the epitope. Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. The peptides are then regarded as leads for the definition of the epitope corresponding to the antibody used to screen the peptide library. For epitope mapping, computational algorithms have also been developed which have been shown to map conformational discontinuous epitopes.

As used herein, the terms “specific binding,” “selective binding,” “selectively binds,” and “specifically binds,” refer to antibody binding to an epitope on a predetermined antigen. Typically, the antibody binds with an equilibrium dissociation constant (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE 2000 instrument (e.g., using recombinant human ILT4 as the analyte and the antibody as the ligand) and binds to the predetermined antigen with an affinity that is at least two-fold greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.”

The term “K_{D},” as used herein, is intended to refer to the dissociation equilibrium constant of a particular antibody-antigen interaction. Typically, the human antibodies of the invention bind to ILT4 with a dissociation equilibrium constant (K_{D}) of approximately 10⁻⁸ M or less, such as less than 10⁻⁹ M or 10⁻¹⁰ M or even lower when determined by surface plasmon resonance (SPR) technology in a BIACORE 2000 instrument (e.g., using recombinant human ILT4 as the analyte and the antibody as the ligand).

The term “kd” as used herein, is intended to refer to the off rate constant for the dissociation of an antibody from the antibody/antigen complex.

The term “ka” as used herein, is intended to refer to the on rate constant for the association of an antibody with the antigen.

The term “EC50,” as used herein, refers to the concentration of an antibody or an antigen binding fragment thereof, which induces a response, either in an in vitro or an in vivo assay, which is 50% of the maximal response, i.e., halfway between the maximal response and the baseline.

As used herein, “isotype” refers to the antibody class (e.g., IgM or IgG1) that is encoded by heavy chain constant region genes. In one embodiment, a human monoclonal antibody of the invention is of the IgG1 isotype. In another embodiment, a human monoclonal antibody of the invention is of the IgG2 isotype.

As used herein, the terms “inhibits” or “blocks” (e.g., referring to inhibition/blocking of binding of HLA-G ligand to ILT4 and/or PD1 to PD-L1 ligand) are used interchangeably and encompass both partial and complete inhibition/blocking. The inhibition/blocking preferably reduces or alters the normal level or type of activity that occurs when binding occurs without inhibition or blocking. Inhibition and blocking are also intended to include any measurable decrease in the binding affinity of HLA-G when in contact with an ILT4 antibody as compared to HLA-G not in contact with an ILT4 antibody, e.g., inhibits binding of HLA-G by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In one embodiment, the ILT4 antibody inhibits binding of HLA-G by at least about 70%. In another embodiment, the ILT4 antibody inhibits binding of HLA-G by at least 80%. Inhibition and blocking are also intended to include any measurable decrease in the binding affinity of PD1 when in contact with an PD-L1 antibody as compared to PD1 not in contact with an PD-L1 antibody, e.g., inhibits binding of PD1 by at least about 10%, 15%, 20%, 25%, 30%, 35%, 40%, 45%, 50%, 55%, 60%, 65%, 70%, 75%, 80%, 85%, 90%, 95%, 96%, 97%, 98%, 99%, or 100%. In one embodiment, the PD-L1 antibody inhibits binding of PD1 by at least about 70%. In another embodiment, the PD-L1 antibody inhibits binding of PD1 by at least 80%.

The term “cross-reacts,” as used herein, refers to the ability of an ILT4 antibody or antigen binding fragment thereof or a PD-L1 antibody or antigen binding fragment thereof of the invention to bind to ILT4 or PD-L1, respectively, from a different species. For example, a ILT4 antibody or antigen binding fragment thereof of the invention that binds human ILT4 may also bind another species of ILT4. Similarly, an PD-L1 antibody or antigen binding fragment thereof of the invention that binds human PD-L1 may also bind another species of PD-L1. As used herein, cross-reactivity is measured by detecting a specific reactivity with purified antigen in binding assays (e.g., SPR, ELISA) or binding to, or otherwise functionally interacting with, cells physiologically expressing ILT4. Methods for determining cross-reactivity include standard binding assays as described herein, for example, by Biacore™ surface plasmon resonance (SPR) analysis using a Biacore™ 2000 SPR instrument (Biacore AB, Uppsala, Sweden), or flow cytometric techniques.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

The term “nucleic acid molecule,” as used herein, is intended to include DNA molecules and RNA molecules. A nucleic acid molecule may be single-stranded or double-stranded, but preferably is double-stranded DNA.

The term “isolated nucleic acid molecule,” as used herein in reference to nucleic acids encoding binding domains, antibodies, or antibody portions (e.g., V_{H}, V_{L}, CDR3) that bind to ILT4 and/or PD-L1, is intended to refer to a nucleic acid molecule in which the nucleotide sequences encoding the antibodies, or antibody portions are free of other nucleotide sequences encoding the antibodies, or antibody portions that bind antigens other than ILT4 and/or PD-L1, which other sequences may naturally flank the nucleic acid in human genomic DNA.

The nucleic acids may be present in whole cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

The nucleic acid molecules of the present invention, while often in a native sequence (except for modified restriction sites and the like), from either cDNA, genomic or mixtures thereof may be mutated, in accordance with standard techniques to provide gene sequences. For coding sequences, these mutations, may affect amino acid sequence as desired. In particular, DNA sequences substantially identical to or derived from native V, D, J, constant, switches and other such sequences described herein are contemplated (where “derived” indicates that a sequence is identical or modified from another sequence).

A nucleic acid is “operably linked” or “operatively linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

The present invention also encompasses “conservative sequence modifications” of any of the sequences described herein, i.e., nucleotide and amino acid sequence modifications which do not abrogate the binding of the VH and VL sequences encoded by the nucleotide sequence or containing the amino acid sequence, to the antigen. Such conservative sequence modifications include conservative nucleotide and amino acid substitutions, as well as, nucleotide and amino acid additions and deletions. For example, modifications can be introduced into the sequences by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an ILT4 antibody is preferably replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)).

In certain embodiments, conservative amino acid sequence modifications refer to at most 1, 2, 3, 4 or 5 conservative amino acid substitutions to the CDR sequences described herein. For example, each such CDR may contain up to 5 conservative amino acid substitutions, e.g., up to (i.e., not more than) 4 conservative amino acid substitutions, e.g., up to (i.e., not more than) 3 conservative amino acid substitutions, e.g., up to (i.e., not more than) 2 conservative amino acid substitutions, or no more than 1 conservative amino acid substitution.

Alternatively, in another embodiment, mutations can be introduced randomly along all or part of an ILT4 or PD-L1 or PD-1 antibody or antigen binding fragment thereof coding sequence, such as by saturation mutagenesis, and the resulting modified ILT4 or PD-L1 or PD-1 antibodies can be screened for binding activity.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98% to 99.5% of the nucleotides. Alternatively, substantial homology exists when the segments will hybridize under selective hybridization conditions, to the complement of the strand.

For amino acids, the term “substantial homology” indicates that two amino acid sequences, or designated sequences thereof, when optimally aligned and compared, are identical, with appropriate amino acid insertions or deletions, in at least about 80% of the amino acids, usually at least about 90% to 95%, and more preferably at least about 98% to 99% or 99.5% of the amino acids.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences of the present invention can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences identical to the nucleic acid molecules of the invention. BLAST protein searches can be performed with the XBLAST program, score=50, wordlength=3 to obtain amino acid sequences identical to the protein molecules of the invention. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

### B. ILT4 Antibodies and Antigen Binding Fragments Thereof

Provided herein are novel ILT4 antibodies (or antigen binding fragments thereof), e.g., humanized antibodies, which are characterized by particular functional features or properties. For example, antibodies (or fragments) of the present invention exhibit one or more of the following properties:
a. blocking ILT4 ligand (e.g., HLA-G ligand) binding to human ILT4;
b. enhancing or increasing cytokine or chemokine release by human macrophages;
c. potentiating the activation effects of LPS and IFNγ on macrophages;
d. promoting M1 macrophage polarization;
e. binding to human ILT4 with an equilibrium dissociation constant Kd of 10⁻⁹ M or less, or alternatively, an equilibrium association constant Ka of 10⁺⁹ M⁻¹ or greater;
f. lack of cross-reactivity with other ILT family members;
g. cross-reactivity with cynomolgus ILT4; and/or
h. inhibiting tumor cells that express ILT4.

An exemplary ILT4 antibody is antibody 7A3 as described herein. In one embodiment, the ILT4 antibody or antigen binding fragment thereof comprises the heavy and light chain CDRs or variable regions of antibody 7A3. In another embodiment, the antibody or antigen binding fragment comprises the CDR1, CDR2, and CDR3 domains of the heavy chain variable region of antibody 7A3 having the sequence set forth in SEQ ID NO:9, and the CDR1, CDR2 and CDR3 domains of the light chain variable region of antibody 7A3 having the sequence set forth in SEQ ID NO:10. In another embodiment, the antibody or antigen binding fragment thereof comprises heavy chain CDR1, CDR2 and CDR3 domains having the sequences set forth in SEQ ID NOs:1, 3, and 5, respectively, or conservative sequence modifications thereof, and light chain CDR1, CDR2 and CDR3 domains having the sequences set forth in SEQ ID NOs:6, 7, and 8, respectively, or conservative sequence modifications thereof. Alternatively, the antibody or antigen binding fragment thereof comprises heavy chain CDR1, CDR2 and CDR3 domains having the sequences set forth in SEQ ID NOs:2, 4, and 5, respectively, or conservative sequence modifications thereof, and light chain CDR1, CDR2 and CDR3 domains having the sequences set forth in SEQ ID NOs:6, 7, and 8, respectively, or conservative sequence modifications thereof. In another embodiment, the antibody or antigen binding fragment thereof comprises a heavy chain variable region having the amino acid sequence set forth in SEQ ID NO:9. Alternatively, the antibody or antigen binding fragment comprises a heavy chain variable region having the amino acid sequence set forth in SEQ ID NO:97, 98, or 99. In another embodiment, the antibody or antigen binding fragment thereof comprises a light chain variable region having the amino acid sequence set forth in SEQ ID NO:10. Alternatively, the antibody or antigen binding fragment comprises a light chain variable region having the amino acid sequence set forth in SEQ ID NO:100, 101, or 102. In another embodiment, the antibody or antigen binding fragment thereof comprises a heavy chain variable region and a light chain variable region, wherein:
(a) the heavy chain variable region comprises an amino acid sequence selected from the group consisting of SEQ ID NOs: 9, 97, 98, and 99, and
(b) the light chain variable region comprises an amino acid sequence selected from the group consisting of SEQ ID NOs:10, 100, 101, and 102.
   For example, the antibody or antigen binding fragment comprises heavy and light chain variable regions having the amino acid sequences set forth in SEQ ID NO:9 and 10.

Another exemplary ILT4 antibody is antibody 7B1 as described herein. In one embodiment, the ILT4 antibody or antigen binding fragment thereof comprises the heavy and light chain CDRs or variable regions of antibody 7B1. In another embodiment, the antibody or antigen binding fragment comprises the CDR1, CDR2, and CDR3 domains of the heavy chain variable region of antibody 7B1 having the sequence set forth in SEQ ID NO:19, and the CDR1, CDR2 and CDR3 domains of the light chain variable region of antibody 7B1 having the sequence set forth in SEQ ID NO:20. In another embodiment, the antibody or antigen binding fragment thereof comprises heavy chain CDR1, CDR2 and CDR3 domains having the sequences set forth in SEQ ID NOs:11, 13, and 15, respectively, or conservative sequence modifications thereof, and light chain CDR1, CDR2 and CDR3 domains having the sequences set forth in SEQ ID NOs:16, 17, and 18, respectively, or conservative sequence modifications thereof. Alternatively, the antibody or antigen binding fragment thereof comprises heavy chain CDR1, CDR2 and CDR3 domains having the sequences set forth in SEQ ID NOs:12, 14, and 15, respectively, or conservative sequence modifications thereof, and light chain CDR1, CDR2 and CDR3 domains having the sequences set forth in SEQ ID NOs:16, 17, and 18, respectively, or conservative sequence modifications thereof. In another embodiment, the antibody or antigen binding fragment thereof comprises a heavy chain variable region having the amino acid sequence set forth in SEQ ID NO:19. Alternatively, the antibody or antigen binding fragment comprises a heavy chain variable region having the amino acid sequence set forth in SEQ ID NO:103, 104, or 105. In another embodiment, the antibody or antigen binding fragment thereof comprises a light chain variable region having the amino acid sequence set forth in SEQ ID NO:20. Alternatively, the antibody or antigen binding fragment comprises a light chain variable region having the amino acid sequence set forth in SEQ ID NO:106, 107, or 108. In another embodiment, the antibody or antigen binding fragment thereof comprises a heavy chain variable region and a light chain variable region, wherein:
(a) the heavy chain variable region comprises an amino acid sequence selected from the group consisting of SEQ ID NOs: 19, 103, 104, and 105, and (b) the light chain variable region comprises an amino acid sequence selected from the group consisting of SEQ ID NOs:20, 106, 107, and 108.

For example, the antibody or antigen binding fragment comprises heavy and light chain variable regions having the amino acid sequences set forth in SEQ ID NO:19 and 20.

The antibody sequences can also be consensus sequences of several antibodies. For example, in one embodiment, the ILT4 antibody or antigen binding fragment comprises a heavy chain variable region CDR1 comprising an amino acid sequence selected from the consensus sequence: G Y T (I,M) H (SEQ ID NO: 21). In another embodiment, the ILT4 antibody or antigen binding fragment comprises a heavy chain variable region CDR2 comprising SEQ ID NO:3. In another embodiment, the ILT4 antibody or antigen binding fragment comprises a heavy chain variable region CDR3 comprising an amino acid sequence selected from the consensus sequence: E R P G G S Q F I Y Y Y (P,A) (M,L) D Y (SEQ ID NO:22). In another embodiment, the ILT4 antigen binding fragment comprises a light chain variable region CDR1 comprising an amino acid sequence selected from the consensus sequence: R A S (A,E) N I Y S Y L A (SEQ ID NO: 23). In another embodiment, the ILT4 antibody or antigen binding fragment comprises a light chain variable region CDR2 comprising an amino acid sequence selected from the consensus sequence: N A (I,D) T L A E (SEQ ID NO: 24). In another embodiment, the ILT4 antibody or antigen binding fragment comprises a light chain variable region CDR3 comprising SEQ ID NO:8.

Given that each of the described antibodies can bind to human ILT4, the V_{H} and V_{L} sequences described herein can be “mixed and matched” to create various ILT4 antibodies or antigen binding fragments thereof. The binding of such “mixed and matched” antibodies to human ILT4 can be tested using the binding assays known in the art and described in the Examples (e.g., ELISAs). For example, ILT4 antibodies and antigen binding fragments thereof of the present invention include combinations of heavy and light chain variable region sequences as set forth in Table 1.

TABLE 1
VH and VL Sequence Combinations (SEQ ID NOs)

| | VH [symbol] | | | | | | | |
|---|---|---|---|---|---|---|---|---|
| VL ▾ | 9 | 97 | 98 | 99 | 19 | 103 | 104 | 105 |
| 10 | 9 × 10 | 97 × 10 | 98 × 10 | 99 × 10 | 19 × 10 | 103 × 10 | 104 × 10 | 105 × 10 |
| 100 | 9 × 100 | 97 × 100 | 98 × 100 | 99 × 100 | 19 × 100 | 103 × 100 | 104 × 100 | 105 × 100 |
| 101 | 9 × 101 | 97 × 101 | 98 × 101 | 99 × 101 | 19 × 101 | 103 × 101 | 104 × 101 | 105 × 101 |
| 102 | 9 × 102 | 97 × 102 | 98 × 102 | 99 × 102 | 19 × 102 | 103 × 102 | 104 × 102 | 105 × 102 |
| 20 | 9 × 20 | 97 × 20 | 98 × 20 | 99 × 20 | 19 × 20 | 103 × 20 | 104 × 20 | 105 × 20 |
| 106 | 9 × 106 | 97 × 106 | 98 × 106 | 99 × 106 | 19 × 106 | 103 × 106 | 104 × 106 | 105 × 106 |
| 107 | 9 × 107 | 97 × 107 | 98 × 107 | 99 × 107 | 19 × 107 | 103 × 107 | 104 × 107 | 105 × 107 |
| 108 | 9 × 108 | 97 × 108 | 98 × 108 | 99 × 108 | 19 × 108 | 103 × 108 | 104 × 108 | 105 × 108 |

Sequences substantially identical to the ILT4 antibodies and antigen binding fragments thereof described herein (e.g., sequences at least 80%, 81%, 82%, 83%, 84%, 85%, 86%, 87%, 88%, 89%, 90%, 91%, 92%, 93%, 94%, 95%, 96%, 97%, 98% or 99% identical to the aforementioned sequences) are also provided. For example, in one embodiment, the ILT4 antibody or antigen binding fragment thereof comprises a heavy chain variable region comprising SEQ ID NO:9, 97, 98, 99, 19, 103, 104, 105, or a sequence at least 80% identical thereto (e.g., at least 80%, 81%, 82%, 83%, 84%, 85%, 86%, 87%, 88%, 89%, 90%, 91%, 92%, 93%, 94%, 95% 96%, 97%, 98%, or 99% identical to the aforementioned sequences). In another embodiment, the ILT4 antibody or antigen binding fragment thereof comprises a light chain variable region comprising SEQ ID NO:10, 100, 101, 102, 20, 106, 107, 108, or a sequence at least 80% identical thereto (e.g., at least 80%, 81%, 82%, 83%, 84%, 85%, 86%, 87%, 88%, 89%, 90%, 91%, 92%, 93%, 94%, 95%, 96%, 97%, 98% or 99% identical to the aforementioned sequences). In another embodiment, the ILT4 antibody (or antigen binding fragment thereof) comprises a (a) heavy chain variable region comprising SEQ ID NO:9, 97, 98, 99, 19, 103, 104, 105, or a sequence at least 80% identical thereto (e.g., at least 80%, 81%, 82%, 83%, 84%, 85%, 86%, 87%, 88%, 89%, 90%, 91%, 92%, 93%, 94%, 95%, 96%, 97%, 98% or 99% identical to the aforementioned sequences) and (b) light chain variable region comprising SEQ ID NO: 10, 100, 101, 102, 20, 106, 107, or 108, or a sequence at least 80% identical thereto (e.g., at least 80%, 81%, 82%, 83%, 84%, 85%, 86%, 87%, 88%, 89%, 90%, 91%, 92%, 93%, 94%, 95%, 96%, 97%, 98% or 99% identical to the aforementioned sequences). For example, the ILT4 antibody or antigen binding fragment thereof comprises SEQ ID NO: 9, or a sequence at least 80% identical thereto (e.g., at least 80%, 81%, 82%, 83%, 84%, 85%, 86%, 87%, 88%, 89%, 90%, 91%, 92%, 93%, 94%, 95%, 96%, 97%, 98% or 99% identical thereto) and SEQ ID NO:19, or a sequence at least 80% identical thereto (e.g., at least 80%, 81%, 82%, 83%, 84%, 85%, 86%, 87%, 88%, 89%, 90%, 91%, 92%, 93%, 94%, 95%, 96%, 97%, 98% or 99% identical thereto). Alternatively, the ILT4 antibody or antigen binding fragment thereof comprises SEQ ID NO:10, or a sequence at least 80% identical thereto (e.g., at least 80%, 81%, 82%, 83%, 84%, 85%, 86%, 87%, 88%, 89%, 90%, 91%, 92%, 93%, 94%, 95%, 96%, 97%, 98% or 99% identical thereto) and SEQ ID NO:20, or a sequence at least 80% identical thereto (e.g., at least 80%, 81%, 82%, 83%, 84%, 85%, 86%, 87%, 88%, 89%, 90%, 91%, 92%, 93%, 94%, 95%, 96%, 97%, 98% or 99% identical thereto).

Other exemplary antibodies include ILT4 antibodies and antigen binding fragments thereof that compete for binding with any of the ILT4 antibody or fragments thereof described herein, or that bind the same epitope as any of the ILT4 antibody or fragments thereof described herein. For example, in one embodiment, the ILT4 antibody or antigen binding fragment thereof competes for binding to ILT4 with antibody 7A3 (or an antibody having the heavy and light chain CDRs and/or heavy and light chain variable region sequences corresponding to antibody 7A3). In another embodiment, the ILT4 antibody or antigen binding fragment thereof competes for binding to ILT4 with antibody 7B1 (or an antibody having the heavy and light chain CDRs and/or heavy and light chain variable region sequences corresponding to antibody 7B1). In another embodiment, the ILT4 antibody or antigen binding fragment thereof binds to the same epitope on ILT4 as antibody 7A3 (or an antibody having the heavy and light chain CDRs and/or heavy and light chain variable region sequences corresponding to antibody 7A3). In another embodiment, the ILT4 antibody or antigen binding fragment thereof binds to the same epitope on ILT4 as antibody 7B1 (or an antibody having the heavy and light chain CDRs and/or heavy and light chain variable region sequences corresponding to antibody 7B1).

In one embodiment, the ILT4 antibody, or antigen binding fragment thereof, comprises heavy chain variable region CDR1, CDR2 and CDR3 as set forth in SEQ ID NOs:1, 3, and 5, respectively, and light chain variable region CDR1, CDR2 and CDR3 as set forth in SEQ ID NOs:6, 7, and 8, respectively. Alternatively, the ILT4 antibody, or antigen binding fragment thereof, comprises heavy chain variable region CDR1, CDR2 and CDR3 as set forth in SEQ ID NOs:2, 4, and 5, respectively, and light chain variable region CDR1, CDR2 and CDR3 as set forth in SEQ ID NOs:6, 7, and 8, respectively. In another embodiment, the ILT4 antibody, or antigen binding fragment thereof, comprises a heavy chain variable region comprising SEQ ID NO:9 and a light chain variable region comprising SEQ ID NO:19 or sequences at least 80% identical to the aforementioned sequences (e.g., at least 80%, 81%, 82%, 83%, 84%, 85%, 86%, 87%, 88%, 89%, 90%, 91%, 92%, 93%, 94%, 95%, 96%, 97%, 98% or 99% identical).

In another embodiment, the ILT4 antibody, or antigen binding fragment thereof, comprises heavy chain variable region CDR1, CDR2 and CDR3 as set forth in SEQ ID NOs:11, 13, and 15, respectively, and light chain variable region CDR1, CDR2 and CDR3 as set forth in SEQ ID NOs:16, 17, and 18, respectively. Alternatively, the ILT4 antibody, or antigen binding fragment thereof, comprises heavy chain variable region CDR1, CDR2 and CDR3 as set forth in SEQ ID NOs:12, 14, and 15, respectively, and light chain variable region CDR1, CDR2 and CDR3 as set forth in SEQ ID NOs:16, 17, and 18, respectively. In another embodiment, the ILT4 antibody, or antigen binding fragment thereof, comprises a heavy chain variable region comprising SEQ ID NO:10 and a light chain variable region comprising SEQ ID NO:20 or sequences at least 80% identical to the aforementioned sequences (e.g., at least 80%, 81%, 82%, 83%, 84%, 85%, 86%, 87%, 88%, 89%, 90%, 91%, 92%, 93%, 94%, 95%, 96%, 97%, 98% or 99% identical).

### C. PD-L1 Antibodies and Antigen Binding Fragments Thereof

Provided herein are PD-L1 antibodies and antigen binding fragments thereof for use with the ILT4 antibodies or antigen binding fragments of the present invention, e.g., in bispecific and multispecific constructs, as well as methods of treatment.

An exemplary PD-L1 antibody is antibody 7H7 as described herein. In one embodiment, the PD-L1 antibody or antigen binding fragment thereof comprises the heavy and light chain CDRs or variable regions of antibody 7H7. In another embodiment, the antibody or antigen binding fragment thereof comprises the CDR1, CDR2, and CDR3 domains of the heavy chain variable region of antibody 7H7 having the sequence set forth in SEQ ID NO:77, and the CDR1, CDR2 and CDR3 domains of the light chain variable region of antibody 7H7 having the sequence set forth in SEQ ID NO:78. In another embodiment, the antibody or antigen binding fragment thereof comprises heavy chain CDR1, CDR2 and CDR3 domains having the sequences set forth in SEQ ID NOs:29, 30, and 31, respectively, or conservative sequence modifications thereof, and light chain CDR1, CDR2 and CDR3 domains having the sequences set forth in SEQ ID NOs:32, 33, and 34, respectively, or conservative sequence modifications thereof. In another embodiment, the antibody or antigen binding fragment thereof comprises a heavy chain variable region having the amino acid sequence set forth in SEQ ID NO:77. In another embodiment, the antibody or antigen binding fragment thereof comprises a heavy chain variable region having the amino acid sequence set forth in SEQ ID NO:77. In another embodiment, the antibody or antigen binding fragment thereof comprises heavy and light chain variable regions having the amino acid sequences set forth in SEQ ID NO:77 and SEQ ID NO:78, respectively.

Another exemplary PD-L1 antibody is antibody 1B3 as described herein. In one embodiment, the PD-L1 antibody or antigen binding fragment thereof comprises the heavy and light chain CDRs or variable regions of antibody 1B3. In another embodiment, the antibody or antigen binding fragment thereof comprises the CDR1, CDR2, and CDR3 domains of the heavy chain variable region of antibody 1B3 having the sequence set forth in SEQ ID NO:79, and the CDR1, CDR2 and CDR3 domains of the light chain variable region of antibody 1B3 having the sequence set forth in SEQ ID NO:80. In another embodiment, the antibody or antigen binding fragment thereof comprises heavy chain CDR1, CDR2 and CDR3 domains having the sequences set forth in SEQ ID NOs:35, 36, and 37, respectively, or conservative sequence modifications thereof, and light chain CDR1, CDR2 and CDR3 domains having the sequences set forth in SEQ ID NOs:38, 39, and 40, respectively, or conservative sequence modifications thereof. In another embodiment, the antibody or antigen binding fragment thereof comprises a heavy chain variable region having the amino acid sequences set forth in SEQ ID NO:79. In another embodiment, the antibody or antigen binding fragment thereof comprises a light chain variable region having the amino acid sequences set forth in SEQ ID NO:80. In another embodiment, the antibody or antigen binding fragment thereof comprises heavy and light chain variable regions having the amino acid sequences set forth in SEQ ID NO:79 and SEQ ID NO:80, respectively.

Another exemplary PD-L1 antibody is antibody 3B6 as described herein. In one embodiment, the PD-L1 antibody or antigen binding fragment thereof comprises the heavy and light chain CDRs or variable regions of antibody 3B6. In another embodiment, the antibody or antigen binding fragment thereof comprises the CDR1, CDR2, and CDR3 domains of the heavy chain variable region of antibody 3B6 having the sequence set forth in SEQ ID NO:81, and the CDR1, CDR2 and CDR3 domains of the light chain variable region of antibody 3B6 having the sequence set forth in SEQ ID NO:82. In another embodiment, the antibody or antigen binding fragment thereof comprises heavy chain CDR1, CDR2 and CDR3 domains having the sequences set forth in SEQ ID NOs:41, 42, and 43, respectively, or conservative sequence modifications thereof, and light chain CDR1, CDR2 and CDR3 domains having the sequences set forth in SEQ ID NOs:44, 45, and 46, respectively, or conservative sequence modifications thereof. In another embodiment, the antibody or antigen binding fragment thereof comprises a heavy chain variable region having the amino acid sequence set forth in SEQ ID NO:81. In another embodiment, the antibody or antigen binding fragment thereof comprises a light chain variable region having the amino acid sequence set forth in SEQ ID NO:82. In another embodiment, the antibody or antigen binding fragment n thereof comprises heavy and light chain variable regions having the amino acid sequences set forth in SEQ ID NO:81 and SEQ ID NO:82, respectively.

Another exemplary PD-L1 antibody is antibody 8B1 as described herein. In one embodiment, the PD-L1 antibody or antigen binding fragment thereof comprises the heavy and light chain CDRs or variable regions of antibody 8B1. In another embodiment, the antibody or antigen binding fragment thereof comprises the CDR1, CDR2, and CDR3 domains of the heavy chain variable region of antibody 8B1 having the sequence set forth in SEQ ID NO:83, and the CDR1, CDR2 and CDR3 domains of the light chain variable region of antibody 8B1 having the sequence set forth in SEQ ID NO:84. In another embodiment, the antibody or antigen binding fragment thereof comprises heavy chain CDR1, CDR2 and CDR3 domains having the sequences set forth in SEQ ID NOs:47, 48, and 49, respectively, or conservative sequence modifications thereof, and light chain CDR1, CDR2 and CDR3 domains having the sequences set forth in SEQ ID NOs:50, 51, and 52, respectively, or conservative sequence modifications thereof. In another embodiment, the antibody or antigen binding fragment thereof comprises a heavy chain variable region having the amino acid sequences set forth in SEQ ID NO:83. In another embodiment, the antibody or antigen binding fragment thereof comprises a light chain variable region having the amino acid sequences set forth in SEQ ID NO:84. In another embodiment, the antibody or antigen binding fragment thereof comprises heavy and light chain variable regions having the amino acid sequences set forth in SEQ ID NO:83 and SEQ ID NO:84, respectively.

Another exemplary PD-L1 antibody is antibody 4A3 as described herein. In one embodiment, the PD-L1 antibody or antigen binding fragment thereof comprises the heavy and light chain CDRs or variable regions of antibody 4A3. In another embodiment, the antibody or antigen binding fragment thereof comprises the CDR1, CDR2, and CDR3 domains of the heavy chain variable region of antibody 4A3 having the sequence set forth in SEQ ID NO:85, and the CDR1, CDR2 and CDR3 domains of the light chain variable region of antibody 4A3 having the sequence set forth in SEQ ID NO:86. In another embodiment, the antibody or antigen binding fragment thereof comprises heavy chain CDR1, CDR2 and CDR3 domains having the sequences set forth in SEQ ID NOs:53, 54, and 55, respectively, or conservative sequence modifications thereof, and light chain CDR1, CDR2 and CDR3 domains having the sequences set forth in SEQ ID NOs:56, 57, and 58, respectively, or conservative sequence modifications thereof. In another embodiment, the antibody or antigen binding fragment thereof comprises a heavy chain variable region having the amino acid sequence set forth in SEQ ID NO:85. In another embodiment, the antibody or antigen binding fragment thereof comprises a light chain variable region having the amino acid sequence set forth in SEQ ID NO:86. In another embodiment, the antibody or antigen binding fragment thereof comprises heavy and light chain variable regions having the amino acid sequences set forth in SEQ ID NO:85 and SEQ ID NO:86, respectively.

Another exemplary PD-L1 antibody is antibody 9H9 as described herein. In one embodiment, the PD-L1 antibody or antigen binding fragment thereof comprises the heavy and light chain CDRs or variable regions of antibody 9H9. In another embodiment, the antibody or antigen binding fragment thereof comprises the CDR1, CDR2, and CDR3 domains of the heavy chain variable region of antibody 9H9 having the sequence set forth in SEQ ID NO:87, and the CDR1, CDR2 and CDR3 domains of the light chain variable region of antibody 9H9 having the sequence set forth in SEQ ID NO:88. In another embodiment, the antibody or antigen binding fragment thereof comprises heavy chain CDR1, CDR2 and CDR3 domains having the sequences set forth in SEQ ID NOs:59, 60, and 61, respectively, or conservative sequence modifications thereof, and light chain CDR1, CDR2 and CDR3 domains having the sequences set forth in SEQ ID NOs:62, 63, and 64, respectively, or conservative sequence modifications thereof. In another embodiment, the antibody or antigen binding fragment thereof comprises a heavy chain variable region having the amino acid sequence set forth in SEQ ID NO:87. In another embodiment, the antibody or antigen binding fragment thereof comprises a light chain variable region having the amino acid sequence set forth in SEQ ID NO:88. In another embodiment, the antibody or antigen binding fragment thereof comprises heavy and light chain variable regions having the amino acid sequences set forth in SEQ ID NO:87 and SEQ ID NO:88, respectively.

The antibody sequences can also be consensus sequences of several antibodies. For example, in one embodiment, the PD-L1 antigen binding fragment comprises a heavy chain variable region CDR1 comprising an amino acid sequence selected from the consensus sequence: (T,S)(S,Y,H)WMS (SEQ ID NO:167). In another embodiment, the PD-L1 antigen binding fragment comprises a heavy chain variable region CDR2 comprising SEQ ID NO:168. In another embodiment, the PD-L1 antigen binding fragment comprises a heavy chain variable region CDR3 comprising SEQ ID NO:169. In another embodiment, the PD-L1 antigen binding fragment comprises a light chain variable region CDR1 comprising SEQ ID NO:170. In another embodiment, the PD-L1 antigen binding fragment comprises a light chain variable region CDR2 comprising SEQ ID NO:171. In another embodiment, the PD-L1 antigen binding fragment comprises a light chain variable region CDR3 comprising SEQ ID NO:172.

Sequences substantially identical to the PD-L1 antibodies and antigen binding fragments thereof described herein (e.g., at least 80%, 85%, 90%, 91%, 92%, 93%, 94%, 95%, 96%, 97%, 98% or 99% identical to the aforementioned sequences), are also encompassed by the invention. In one embodiment, the PD-L1 antigen binding fragment comprises a heavy chain variable region comprising SEQ ID NO:77, SEQ ID NO:79, SEQ ID NO:81, SEQ ID NO:83, SEQ ID NO:85, SEQ ID NO:87, or a sequence at least 90% identical thereto (e.g., at least 90%, 91%, 92%, 93%, 94%, 95%, 96%, 97%, 98% or 99% identical to the aforementioned sequences). In another embodiment, the PD-L1 antigen binding fragment comprises a light chain variable region comprising SEQ ID NO:78, SEQ ID NO:80, SEQ ID NO:82, SEQ ID NO:84, SEQ ID NO:86, SEQ ID NO:88 or a sequence at least 90% identical thereto (e.g., at least 90%, 91%, 92%, 93%, 94%, 95%, 96%, 97%, 98% or 99% identical to the aforementioned sequences). In another embodiment, the PD-L1 antigen binding fragment comprises a heavy chain variable region comprising SEQ ID NO:77 and a light chain variable region comprising SEQ ID NO:78 or sequences at least 90% identical thereto (e.g., at least 90%, 91%, 92%, 93%, 94%, 95%, 96%, 97%, 98% or 99% identical to the aforementioned sequences). In another embodiment, the PD-L1 antigen binding fragment comprises a heavy chain variable region comprising SEQ ID NO:79 and a light chain variable region comprising SEQ ID NO:80 or sequences at least 90% identical thereto (e.g., at least 90%, 91%, 92%, 93%, 94%, 95%, 96%, 97%, 98% or 99% identical to the aforementioned sequences). In another embodiment, the PD-L1 antigen binding fragment comprises a heavy chain variable region comprising SEQ ID NO:81 and a light chain variable region comprising SEQ ID NO:82 or sequences at least 90% identical thereto (e.g., at least 90%, 91%, 92%, 93%, 94%, 95%, 96%, 97%, 98% or 99% identical to the aforementioned sequences). In another embodiment, the PD-L1 antigen binding fragment comprises a heavy chain variable region comprising SEQ ID NO:83 and a light chain variable region comprising SEQ ID NO:84 or sequences at least 90% identical thereto (e.g., at least 90%, 91%, 92%, 93%, 94%, 95%, 96%, 97%, 98% or 99% identical to the aforementioned sequences). In another embodiment, the PD-L1 antigen binding fragment comprises a heavy chain variable region comprising SEQ ID NO:85 and a light chain variable region comprising SEQ ID NO:86 or sequences at least 90% identical thereto (e.g., at least 90%, 91%, 92%, 93%, 94%, 95%, 96%, 97%, 98% or 99% identical to the aforementioned sequences). In another embodiment, the PD-L1 antigen binding fragment comprises a heavy chain variable region comprising SEQ ID NO:87 and a light chain variable region comprising SEQ ID NO:88 or sequences at least 90% identical thereto (e.g., at least 90%, 91%, 92%, 93%, 94%, 95%, 96%, 97%, 98% or 99% identical to the aforementioned sequences).

Other exemplary antibodies include PD-L1 antibodies and antigen binding fragments thereof that compete for binding with any of the PD-L1 antibodies or antigen binding fragments thereof as described herein, or that bind the same epitope as any of the PD-L1 antibodies or antigen binding fragments thereof as described herein. In one embodiment, the PD-L1 antibody or antigen binding fragment thereof competes for binding to PD-L1 with antibody 7H7 (or an antibody having the heavy and light chain CDRs and/or heavy and light chain variable region sequences corresponding to antibody 7H7). In another embodiment, the PD-L1 antibody or antigen binding fragment thereof binds to the same epitope on PD-L1 as antibody 7H7 (or an antibody having the heavy and light chain CDRs and/or heavy and light chain variable region sequences corresponding to antibody 7H7).

In another embodiment, the PD-L1 antibody or antigen binding fragment thereof competes for binding to PD-L1 with antibody 1B3 (or an antibody having the heavy and light chain CDRs and/or heavy and light chain variable region sequences corresponding to antibody 1B3). In another embodiment, the PD-L1 antibody or antigen binding fragment thereof binds to the same epitope on PD-L1 as antibody 1B3 (or an antibody having the heavy and light chain CDRs and/or heavy and light chain variable region sequences corresponding to antibody 1B3).

In another embodiment, the PD-L1 antibody or antigen binding fragment thereof competes for binding to PD-L1 with antibody 3B6 (or an antibody having the heavy and light chain CDRs and/or heavy and light chain variable region sequences corresponding to antibody 3B6). In another embodiment, the PD-L1 antibody or antigen binding fragment thereof binds to the same epitope on PD-L1 as antibody 3B6 (or an antibody having the heavy and light chain CDRs and/or heavy and light chain variable region sequences corresponding to antibody 3B6).

In another embodiment, the PD-L1 antibody or antigen binding fragment thereof competes for binding to PD-L1 with antibody 8B1 (or an antibody having the heavy and light chain CDRs and/or heavy and light chain variable region sequences corresponding to antibody 8B1). In another embodiment, the PD-L1 antibody or antigen binding fragment thereof binds to the same epitope on PD-L1 as antibody 8B1 (or an antibody having the heavy and light chain CDRs and/or heavy and light chain variable region sequences corresponding to antibody 8B1).

In another embodiment, the PD-L1 antibody or antigen binding fragment thereof competes for binding to PD-L1 with antibody 4A3 (or an antibody having the heavy and light chain CDRs and/or heavy and light chain variable region sequences corresponding to antibody 4A3). In another embodiment, the PD-L1 antibody or antigen binding fragment thereof binds to the same epitope on PD-L1 as antibody 4A3 (or an antibody having the heavy and light chain CDRs and/or heavy and light chain variable region sequences corresponding to antibody 4A3).

In another embodiment, the PD-L1 antibody or antigen binding fragment thereof competes for binding to PD-L1 with antibody 9H9 (or an antibody having the heavy and light chain CDRs and/or heavy and light chain variable region sequences corresponding to antibody 9H9). In another embodiment, the PD-L1 antibody or antigen binding fragment thereof binds to the same epitope on PD-L1 as antibody 9H9 (or an antibody having the heavy and light chain CDRs and/or heavy and light chain variable region sequences corresponding to antibody 9H9).

In another embodiment, the PD-L1 antigen binding fragment is an PD-L1 antibody, or antigen binding fragment thereof. In one embodiment, the PD-L1 antibody, or antigen binding fragment thereof, comprises heavy chain variable region CDR1, CDR2 and CDR3 as set forth in SEQ ID NOs:29, 30, and 31, respectively, and light chain variable region CDR1, CDR2 and CDR3 as set forth in SEQ ID NOs:32, 33, and 34, respectively. In another embodiment, the PD-L1 antibody, or antigen binding fragment thereof, comprises a heavy chain variable region comprising SEQ ID NO:77 and a light chain variable region comprising SEQ ID NO:78 or sequences at least 90% identical thereto (e.g., at least 90%, 91%, 92%, 93%, 94%, 95%, 96%, 97%, 98% or 99% identical to the aforementioned sequences). In another embodiment, the PD-L1 antibody, or antigen binding fragment thereof, comprises heavy chain variable region CDR1, CDR2 and CDR3 as set forth in SEQ ID NOs:35, 36, and 37, respectively, and light chain variable region CDR1, CDR2 and CDR3 as set forth in SEQ ID NOs:38, 39, and 40, respectively. In another embodiment, the PD-L1 antibody, or antigen binding fragment thereof, comprises a heavy chain variable region comprising SEQ ID NO:79 and a light chain variable region comprising SEQ ID NO:80 or sequences at least 90% identical thereto (e.g., at least 90%, 91%, 92%, 93%, 94%, 95%, 96%, 97%, 98% or 99% identical to the aforementioned sequences). In another embodiment, the PD-L1 antibody, or antigen binding fragment thereof, comprises heavy chain variable region CDR1, CDR2 and CDR3 as set forth in SEQ ID NOs:41, 42, and 43, respectively, and light chain variable region CDR1, CDR2 and CDR3 as set forth in SEQ ID NOs:44, 45, and 46, respectively. In another embodiment, the PD-L1 antibody, or antigen binding fragment thereof, comprises a heavy chain variable region comprising SEQ ID NO:81 and a light chain variable region comprising SEQ ID NO:82 or sequences at least 90% identical thereto (e.g., at least 90%, 91%, 92%, 93%, 94%, 95%, 96%, 97%, 98% or 99% identical to the aforementioned sequences). In another embodiment, the PD-L1 antibody, or antigen binding fragment thereof, comprises heavy chain variable region CDR1, CDR2 and CDR3 as set forth in SEQ ID NOs:47, 48, and 49, respectively, and light chain variable region CDR1, CDR2 and CDR3 as set forth in SEQ ID NOs:50, 51, and 52, respectively. In another embodiment, the PD-L1 antibody, or antigen binding fragment thereof, comprises a heavy chain variable region comprising SEQ ID NO:83 and a light chain variable region comprising SEQ ID NO:84 or sequences at least 90% identical thereto (e.g., at least 90%, 91%, 92%, 93%, 94%, 95%, 96%, 97%, 98% or 99% identical to the aforementioned sequences). In another embodiment, the PD-L1 antibody, or antigen binding fragment thereof, comprises heavy chain variable region CDR1, CDR2 and CDR3 as set forth in SEQ ID NOs:53, 54, and 55, respectively, and light chain variable region CDR1, CDR2 and CDR3 as set forth in SEQ ID NOs:56, 57, and 58, respectively. In another embodiment, the PD-L1 antibody, or antigen binding fragment thereof, comprises a heavy chain variable region comprising SEQ ID NO:85 and a light chain variable region comprising SEQ ID NO:86 or sequences at least 90% identical thereto (e.g., at least 90%, 91%, 92%, 93%, 94%, 95%, 96%, 97%, 98% or 99% identical to the aforementioned sequences). In another embodiment, the PD-L1 antibody, or antigen binding fragment thereof, comprises heavy chain variable region CDR1, CDR2 and CDR3 as set forth in SEQ ID NOs:59, 60, and 61, respectively, and light chain variable region CDR1, CDR2 and CDR3 as set forth in SEQ ID NOs:62, 63, and 64, respectively. In another embodiment, the PD-L1 antibody, or antigen binding fragment thereof, comprises a heavy chain variable region comprising SEQ ID NO:87 and a light chain variable region comprising SEQ ID NO:88 or sequences at least 90% identical thereto (e.g., at least 90%, 91%, 92%, 93%, 94%, 95%, 96%, 97%, 98% or 99% identical to the aforementioned sequences).

In another embodiment, the PD-L1 antibody, or antigen binding fragment thereof, has one or more of the following functional features: (a) blocks binding of PD1 to PD-L1 (e.g., partially or completely), (b) induces NFAT pathway activation, and/or (c) induces a mixed lymphocyte reaction.

### D. Binding Agents

Additional binding agents (e.g., ligands, receptor/trap sequences, or antibodies and antigen binding fragments thereof) for use with the ILT4 antibodies or antigen binding fragments of the present invention include, e.g., binding agents which bind to an immune checkpoint molecule (such as PD-1, PD-L1 CTLA-4, LAG-3, TIGIT, TIM-3, VISTA, AXL, ILT2, or ILT3), an immune costimulatory molecule (such as CD27, CD40, 4-1BB, OX40, or GITR), or a tumor antigen (such as HER2, EGFR, ErB3, or CD24). Exemplary binding agents include antibodies or antigen binding fragments thereof which bind to human PD-1, e.g., a PD-1 antagonist. An exemplary PD-1 antibody is nivolumab (referred to as 5C4 in WO 2006/121168; also known as BMS-936558, MDX-1106 or ONO-4538). Particular exemplary binding agents include PD-L1 and PD-1 antibodies (or antigen binding fragments thereof) such as durvalumab, pembrolizumab (Keytruda®), cemiplimab (Libtaylo®), avelumab (Bavencio®), durvalumab (Imfinzi@), and atezolizumab (Tecentriq®).

### E. Bispecific and Multispecific Constructs

Also provided herein are bispecific constructs comprising an ILT4 antibody or antigen binding fragment thereof linked to a second binding agent, e.g., a second binding agent that binds to an immune checkpoint molecule (such as PD-1, PD-L1 CTLA-4, LAG-3 TIGIT, TIM-3, VISTA, AXL, ILT2, or ILT3), an immune costimulatory molecule (such as CD27, CD40, 4-1BB, OX40, or GITR), or a tumor antigen (such as HER2, EGFR, ErB3, or CD24), for example, a bispecific construct comprising an ILT4 antibody (or antigen binding fragment thereof) linked to a PD-L1 or PD-1 antibody (or antigen binding fragment thereof). Such bispecific constructs linked to one or more additional binding agent to form multispecific constructs also are described.

A “bispecific” or “bifunctional” construct is an artificial hybrid having two different binding domain (e.g., heavy/light chain) pairs and two different binding sites. Bispecific constructs can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

For chemical conjugation, suitable reagents and methods are known in the art for coupling two or more moieties, in particular two or more antibodies, or fragments thereof, together. A variety of coupling or crosslinking agents are commercially available and can be used to conjugate the ILT4 antibody or antigen binding fragment thereof and PD-L1 or PD-1 antibody or antigen binding fragment thereof. Non-limiting examples include Sulfo-SMCC, protein A, carboiimide, dimaleimide, dithio-bis-nitrobenzoic acid (DTNB), and N-succinimidyl-3-(2-pyridyldithio) propionate (SPDP). Sulfo-SMCC, SPDP and DTNB are preferred agents, with Sulfo-SMCC being particularly preferred. Other suitable procedures for crosslinking components (e.g., antibodies or antigen binding fragments thereof) with cross-linking agents are known in the art. See e.g., Karpovsky, B. et al., (1984) J. Exp. Med. 160:1686; Liu, M. A. et al., (1985) Proc. Natl. Acad. Sci USA 82:8648; Segal, D. M. and Perez, P., U.S. Pat. No. 4,676,980; and Brennan, M. (1986) Biotechniques 4:424.

For genetic engineering, nucleic acid molecules encoding the ILT4 antibody or antigen binding fragment thereof can be inserted into an appropriate expression vector using standard recombinant DNA techniques. A nucleic acid molecule(s) encoding the PD-L1 or PD-1 antibody or antigen binding fragment thereof also can be inserted into the same expression vector, such that it is operatively linked (e.g., in-frame cloning) to the ILT4 antibody or antigen binding fragment thereof, thereby resulting in an expression vector that encodes a fusion protein that is the bispecific construct. Preferably, the PD-L1 or PD-1 antibody or antigen binding fragment thereof is operatively linked to the C-terminal region of the heavy chain of the ILT4 antibody or antigen binding fragment thereof. Other suitable expression vectors and cloning strategies for preparing the bispecific constructs described herein are known in the art.

For expression of the bispecific constructs in host cells, the coding regions of the antibodies or antigen binding fragments thereof are combined with cloned promoter, leader sequence, translation initiation, leader sequence, constant region, 3′ untranslated, polyadenylation, and transcription termination, sequences to form expression vector constructs. These constructs can be used to express, for example, full length human IgG₁κ or IgG₄κ antibodies. Fully human, humanized and chimeric antibodies used in the bispecific constructs described herein also include IgG2, IgG3, IgE, IgA, IgM, and IgD antibodies. Similar plasmids can be constructed for expression of other heavy chain isotypes, or for expression of antibodies comprising lambda light chains.

Following preparation of an expression vector encoding the bispecific construct, the bispecific construct can be expressed recombinantly in a host cell using standard transfection methods. For example, in one embodiment, nucleic acid encoding the bispecific construct can be ligated into an expression vector, such as a eukaryotic expression plasmid, such as used by GS gene expression system disclosed in WO 87/04462, WO 89/01036 and EP 338 841 or other expression systems well known in the art. The purified plasmid with the cloned bispecific construct gene can be introduced in eukaryotic host cells, such as CHO-cells or NSO-cells or alternatively other eukaryotic cells like a plant derived cells, fungi or yeast cells. The method used to introduce these genes could be methods described in the art, such as electroporation, lipofectin, lipofectamine or other. After introducing the expression vector in the host cells, cells expressing the bispecific construct can be identified and selected. These cells represent the transfectomas that can then be amplified for their expression level and upscaled to produce bispecific constructs. Alternatively, these cloned bispecific constructs can be expressed in other expression systems, such as E. coli or in complete organisms or can be synthetically expressed. Recombinant bispecific constructs can be isolated and purified from these culture supernatants and/or cells.

A bispecific construct of the invention, whether prepared by chemical conjugation or by genetic engineering, can be isolated and purified using one or more methodologies for protein purification well established in the art. Preferred methods for isolation and purification include, but are not limited to, gel filtration chromatography, affinity chromatography, anion-exchange chromatography and the like. A particularly preferred method is gel filtration chromatography, e.g., using a Superdex 200 column. Isolated and purified bispecific constructs can be evaluated using standard methods such as SDS-PAGE analysis.

Accordingly, in one embodiment, the ILT4 antibody or antigen binding fragment thereof is genetically fused to a PD-L1 or PD-1 antibody or antigen binding fragment thereof. In another embodiment, the ILT4 antibody or antigen binding fragment thereof and the PD-L1 antibody or antigen binding fragment thereof are chemically conjugated. In one embodiment, the PD-L1 or PD-1 antibody or antigen binding fragment thereof further comprises a human IgG1 constant domain. In another embodiment, the ILT4 antibody or antigen binding fragment thereof is linked to the C-terminus of the heavy chain of the PD-L1 or PD-1 antibody or antigen binding fragment thereof. In another embodiment, the ILT4 antigen binding fragment thereof is a scFv. In another embodiment, the ILT4 antibody or antigen binding fragment thereof further comprises a human IgG1 constant domain. In another embodiment, the PD-L1 or PD-1 antibody or antigen binding fragment thereof is linked to the C-terminus of the heavy chain of the ILT4 antibody or antigen binding fragment thereof. In another embodiment, the PD-L1 or PD-1 antigen binding fragment thereof is a scFv.

In another aspect, the bispecific and multispecific constructs of the present invention comprise a modified human Fc domain (e.g., a modified IgG1 Fc domain), for example, (a) a modified human IgG1 Fc domain which comprises non-naturally occurring amino acids 234A, 235Q and 322Q as numbered by the EU index as set forth in Kabat, (b) a modified human IgG1 Fc domain which comprises non-naturally occurring amino acids 252Y, 254T and 256E as numbered by the EU index as set forth in Kabat, and/or (c) a modified human IgG1 Fc domain which comprises non-naturally occurring amino acids 234A, 235Q and 322Q as numbered by the EU index as set forth in Kabat.

Exemplary bispecific constructs are set forth below in Tables 2 and 3, wherein the antibody or antigen binding fragments thereof are defined by CDR sequences (Table 2) or variable region sequences (Table 3).

TABLE 2
Exemplary Bispecific Constructs (CDRs)

| Bispecific | ILT4 Heavy Chain | ILT4 Light Chain | PD-L1 Heavy Chain | PD-L1 Light Chain |
|---|---|---|---|---|
| Construct | Variable CDRs | Variable CDRs | Variable CDRs | Variable CDRs |
| Consensus | CDR1: SEQ ID NO: 21 | CDR1: SEQ ID NO: 23 | CDR1: SEQ ID NO: 29 | CDR1: SEQ ID NO: 32 |
| ILT4 × 7H7 | CDR2: SEQ ID NO: 2 | CDR2: SEQ ID NO: 24 | CDR2: SEQ ID NO: 30 | CDR2: SEQ ID NO: 33 |
| | CDR3: SEQ ID NO: 22 | CDR3: SEQ ID NO: 8 | CDR3: SEQ ID NO: 31 | CDR3: SEQ ID NO: 34 |
| Consensus | CDR1: SEQ ID NO: 21 | CDR1: SEQ ID NO: 23 | CDR1: SEQ ID NO: 35 | CDR1: SEQ ID NO: 38 |
| ILT4 × 1B3 | CDR2: SEQ ID NO: 2 | CDR2: SEQ ID NO: 24 | CDR2: SEQ ID NO: 36 | CDR2: SEQ ID NO: 39 |
| | CDR3: SEQ ID NO: 22 | CDR3: SEQ ID NO: 8 | CDR3: SEQ ID NO: 37 | CDR3: SEQ ID NO: 40 |
| Consensus | CDR1: SEQ ID NO: 21 | CDR1: SEQ ID NO: 23 | CDR1: SEQ ID NO: 41 | CDR1: SEQ ID NO: 44 |
| ILT4 × 3B6 | CDR2: SEQ ID NO: 2 | CDR2: SEQ ID NO: 24 | CDR2: SEQ ID NO: 42 | CDR2: SEQ ID NO: 45 |
| | CDR3: SEQ ID NO: 22 | CDR3: SEQ ID NO: 8 | CDR3: SEQ ID NO: 43 | CDR3: SEQ ID NO: 46 |
| Consensus | CDR1: SEQ ID NO: 21 | CDR1: SEQ ID NO: 23 | CDR1: SEQ ID NO: 47 | CDR1: SEQ ID NO: 50 |
| ILT4 × 8B1 | CDR2: SEQ ID NO: 2 | CDR2: SEQ ID NO: 24 | CDR2: SEQ ID NO: 48 | CDR2: SEQ ID NO: 51 |
| | CDR3: SEQ ID NO: 22 | CDR3: SEQ ID NO: 8 | CDR3: SEQ ID NO: 49 | CDR3: SEQ ID NO: 52 |
| Consensus | CDR1: SEQ ID NO: 21 | CDR1: SEQ ID NO: 23 | CDR1: SEQ ID NO: 53 | CDR1: SEQ ID NO: 56 |
| ILT4 × 4A3 | CDR2: SEQ ID NO: 2 | CDR2: SEQ ID NO: 24 | CDR2: SEQ ID NO: 54 | CDR2: SEQ ID NO: 57 |
| | CDR3: SEQ ID NO: 22 | CDR3: SEQ ID NO: 8 | CDR3: SEQ ID NO: 55 | CDR3: SEQ ID NO: 58 |
| Consensus | CDR1: SEQ ID NO: 21 | CDR1: SEQ ID NO: 23 | CDR1: SEQ ID NO: 59 | CDR1: SEQ ID NO: 62 |
| ILT4 × 9H9 | CDR2: SEQ ID NO: 2 | CDR2: SEQ ID NO: 24 | CDR2: SEQ ID NO: 60 | CDR2: SEQ ID NO: 63 |
| | CDR3: SEQ ID NO: 22 | CDR3: SEQ ID NO: 8 | CDR3: SEQ ID NO: 61 | CDR3: SEQ ID NO: 64 |
| 7A3 × 7H7 | CDR1: SEQ ID NO: 1 | CDR1: SEQ ID NO: 6 | CDR1: SEQ ID NO: 29 | CDR1: SEQ ID NO: 32 |
| | CDR2: SEQ ID NO: 3 | CDR2: SEQ ID NO: 7 | CDR2: SEQ ID NO: 30 | CDR2: SEQ ID NO: 33 |
| | CDR3: SEQ ID NO: 5 | CDR3: SEQ ID NO: 8 | CDR3: SEQ ID NO: 31 | CDR3: SEQ ID NO: 34 |
| 7A3 × 1B3 | CDR1: SEQ ID NO: 1 | CDR1: SEQ ID NO: 6 | CDR1: SEQ ID NO: 35 | CDR1: SEQ ID NO: 38 |
| | CDR2: SEQ ID NO: 3 | CDR2: SEQ ID NO: 7 | CDR2: SEQ ID NO: 36 | CDR2: SEQ ID NO: 39 |
| | CDR3: SEQ ID NO: 5 | CDR3: SEQ ID NO: 8 | CDR3: SEQ ID NO: 37 | CDR3: SEQ ID NO: 40 |
| 7A3 × 3B6 | CDR1: SEQ ID NO: 1 | CDR1: SEQ ID NO: 6 | CDR1: SEQ ID NO: 41 | CDR1: SEQ ID NO: 44 |
| | CDR2: SEQ ID NO: 3 | CDR2: SEQ ID NO: 7 | CDR2: SEQ ID NO: 42 | CDR2: SEQ ID NO: 45 |
| | CDR3: SEQ ID NO: 5 | CDR3: SEQ ID NO: 8 | CDR3: SEQ ID NO: 43 | CDR3: SEQ ID NO: 46 |
| 7A3 × 8B1 | CDR1: SEQ ID NO: 1 | CDR1: SEQ ID NO: 6 | CDR1: SEQ ID NO: 47 | CDR1: SEQ ID NO: 50 |
| | CDR2: SEQ ID NO: 3 | CDR2: SEQ ID NO: 7 | CDR2: SEQ ID NO: 48 | CDR2: SEQ ID NO: 51 |
| | CDR3: SEQ ID NO: 5 | CDR3: SEQ ID NO: 8 | CDR3: SEQ ID NO: 49 | CDR3: SEQ ID NO: 52 |
| 7A3 × 4A3 | CDR1: SEQ ID NO: 1 | CDR1: SEQ ID NO: 6 | CDR1: SEQ ID NO: 53 | CDR1: SEQ ID NO: 56 |
| | CDR2: SEQ ID NO: 3 | CDR2: SEQ ID NO: 7 | CDR2: SEQ ID NO: 54 | CDR2: SEQ ID NO: 57 |
| | CDR3: SEQ ID NO: 5 | CDR3: SEQ ID NO: 8 | CDR3: SEQ ID NO: 55 | CDR3: SEQ ID NO: 58 |
| 7A3 × 9H9 | CDR1: SEQ ID NO: 1 | CDR1: SEQ ID NO: 6 | CDR1: SEQ ID NO: 59 | CDR1: SEQ ID NO: 62 |
| | CDR2: SEQ ID NO: 3 | CDR2: SEQ ID NO: 7 | CDR2: SEQ ID NO: 60 | CDR2: SEQ ID NO: 63 |
| | CDR3: SEQ ID NO: 5 | CDR3: SEQ ID NO: 8 | CDR3: SEQ ID NO: 61 | CDR3: SEQ ID NO: 64 |
| 7B1 × 7H7 | CDR1: SEQ ID NO: 11 | CDR1: SEQ ID NO: 16 | CDR1: SEQ ID NO: 29 | CDR1: SEQ ID NO: 32 |
| | CDR2: SEQ ID NO: 13 | CDR2: SEQ ID NO: 17 | CDR2: SEQ ID NO: 30 | CDR2: SEQ ID NO: 33 |
| | CDR3: SEQ ID NO: 15 | CDR3: SEQ ID NO: 18 | CDR3: SEQ ID NO: 31 | CDR3: SEQ ID NO: 34 |
| 7B1 × 1B3 | CDR1: SEQ ID NO: 11 | CDR1: SEQ ID NO: 16 | CDR1: SEQ ID NO: 35 | CDR1: SEQ ID NO: 38 |
| | CDR2: SEQ ID NO: 13 | CDR2: SEQ ID NO: 17 | CDR2: SEQ ID NO: 36 | CDR2: SEQ ID NO: 39 |
| | CDR3: SEQ ID NO: 15 | CDR3: SEQ ID NO: 18 | CDR3: SEQ ID NO: 37 | CDR3: SEQ ID NO: 40 |
| 7B1 × 3B6 | CDR1: SEQ ID NO: 11 | CDR1: SEQ ID NO: 16 | CDR1: SEQ ID NO: 41 | CDR1: SEQ ID NO: 44 |
| | CDR2: SEQ ID NO: 13 | CDR2: SEQ ID NO: 17 | CDR2: SEQ ID NO: 42 | CDR2: SEQ ID NO: 45 |
| | CDR3: SEQ ID NO: 15 | CDR3: SEQ ID NO: 18 | CDR3: SEQ ID NO: 43 | CDR3: SEQ ID NO: 46 |
| 7B1 × 8B1 | CDR1: SEQ ID NO: 11 | CDR1: SEQ ID NO: 16 | CDR1: SEQ ID NO: 47 | CDR1: SEQ ID NO: 50 |
| | CDR2: SEQ ID NO: 13 | CDR2: SEQ ID NO: 17 | CDR2: SEQ ID NO: 48 | CDR2: SEQ ID NO: 51 |
| | CDR3: SEQ ID NO: 15 | CDR3: SEQ ID NO: 18 | CDR3: SEQ ID NO: 49 | CDR3: SEQ ID NO: 52 |
| 7B1 × 4A3 | CDR1: SEQ ID NO: 11 | CDR1: SEQ ID NO: 16 | CDR1: SEQ ID NO: 53 | CDR1: SEQ ID NO: 56 |
| | CDR2: SEQ ID NO: 13 | CDR2: SEQ ID NO: 17 | CDR2: SEQ ID NO: 54 | CDR2: SEQ ID NO: 57 |
| | CDR3: SEQ ID NO: 15 | CDR3: SEQ ID NO: 18 | CDR3: SEQ ID NO: 55 | CDR3: SEQ ID NO: 58 |
| 7B1 × 9H9 | CDR1: SEQ ID NO: 11 | CDR1: SEQ ID NO: 16 | CDR1: SEQ ID NO: 59 | CDR1: SEQ ID NO: 62 |
| | CDR2: SEQ ID NO: 13 | CDR2: SEQ ID NO: 17 | CDR2: SEQ ID NO: 60 | CDR2: SEQ ID NO: 63 |
| | CDR3: SEQ ID NO: 15 | CDR3: SEQ ID NO: 18 | CDR3: SEQ ID NO: 61 | CDR3: SEQ ID NO: 64 |

TABLE 3
Exemplary Bispecific Constructs (VRs)

| | ILT4 | ILT4 | PD-L1 | PD-L1 |
|---|---|---|---|---|
| Bispecific | Heavy Chain | Light Chain | Heavy Chain | Light Chain |
| Constructs | Variable Region | Variable Region | Variable Region | Variable Region |
| 7A3 × 7H7 | SEQ ID NO: 9 | SEQ ID NO: 10 | SEQ ID NO: 77 | SEQ ID NO: 78 |
| 7A3 × 1B3 | SEQ ID NO: 9 | SEQ ID NO: 10 | SEQ ID NO: 79 | SEQ ID NO: 80 |
| 7A3 × 3B6 | SEQ ID NO: 9 | SEQ ID NO: 10 | SEQ ID NO: 81 | SEQ ID NO: 82 |
| 7A3 × 8B1 | SEQ ID NO: 9 | SEQ ID NO: 10 | SEQ ID NO: 83 | SEQ ID NO: 84 |
| 7A3 × 4A3 | SEQ ID NO: 9 | SEQ ID NO: 10 | SEQ ID NO: 85 | SEQ ID NO: 86 |
| 7A3 × 9H9 | SEQ ID NO: 9 | SEQ ID NO: 10 | SEQ ID NO: 87 | SEQ ID NO: 88 |
| 7B1 × 7H7 | SEQ ID NO: 19 | SEQ ID NO: 20 | SEQ ID NO: 77 | SEQ ID NO: 78 |
| 7B1 × 1B3 | SEQ ID NO: 19 | SEQ ID NO: 20 | SEQ ID NO: 79 | SEQ ID NO: 80 |
| 7B1 × 3B6 | SEQ ID NO: 19 | SEQ ID NO: 20 | SEQ ID NO: 81 | SEQ ID NO: 82 |
| 7B1 × 8B1 | SEQ ID NO: 19 | SEQ ID NO: 20 | SEQ ID NO: 83 | SEQ ID NO: 84 |
| 7B1 × 4A3 | SEQ ID NO: 19 | SEQ ID NO: 20 | SEQ ID NO: 85 | SEQ ID NO: 86 |
| 7B1 × 9H9 | SEQ ID NO: 19 | SEQ ID NO: 20 | SEQ ID NO: 87 | SEQ ID NO: 88 |
| | SEQ ID NO: 9, 10, | SEQ ID NO: 10, 20, | SEQ ID NO: 77 | SEQ ID NO: 78 |
| | 97, 98, 99, 103, | 100, 101, 102, 106, | | |
| | 104, or 105 | 107, or 108 | | |
| | SEQ ID NO: 9, 10, | SEQ ID NO: 10, 20, | SEQ ID NO: 79 | SEQ ID NO: 80 |
| | 97, 98, 99, 103, | 100, 101, 102, 106, | | |
| | 104, or 105 | 107, or 108 | | |
| | SEQ ID NO: 9, 10, | SEQ ID NO: 10, 20, | SEQ ID NO: 81 | SEQ ID NO: 82 |
| | 97, 98, 99, 103, | 100, 101, 102, 106, | | |
| | 104, or 105 | 107, or 108 | | |
| | SEQ ID NO: 9, 10, | SEQ ID NO: 10, 20, | SEQ ID NO: 83 | SEQ ID NO: 84 |
| | 97, 98, 99, 103, | 100, 101, 102, 106, | | |
| | 104, or 105 | 107, or 108 | | |
| | SEQ ID NO: 9, 10, | SEQ ID NO: 10, 20, | SEQ ID NO: 85 | SEQ ID NO: 86 |
| | 97, 98, 99, 103, | 100, 101, 102, 106, | | |
| | 104, or 105 | 107, or 108 | | |
| | SEQ ID NO: 9, 10, | SEQ ID NO: 10, 20, | SEQ ID NO: 87 | SEQ ID NO: 88 |
| | 97, 98, 99, 103, | 100, 101, 102, 106, | | |
| | 104, or 105 | 107, or 108 | | |

Bispecific and multispecific constructs comprising sequences substantially identical to the aforementioned ILT4 and PD-L1 sequences (i.e., CDR and variable region sequences) also are provided herein (e.g., sequences having conservative sequence modifications and/or sequences at least 80%, 81%, 82%, 83%, 84%, 85%, 86%, 87%, 88%, 89%, 90%, 91%, 92%, 93%, 94%, 95%, 96%, 97%, 98% or 99% identical to the aforementioned sequences).

In another embodiment, the bispecific and multispecific constructs exhibit one or more of the following properties:
a. blocking ILT4 ligand (e.g., HLA-G ligand) binding to human ILT4;
b. enhancing or increasing cytokine or chemokine release by human macrophages;
c. potentiating the activation effects of LPS and IFNγ on macrophages;
d. promoting M1 macrophage polarization;
e. binding to human ILT4 with an equilibrium dissociation constant Kd of 10⁻⁹ M or less, or alternatively, an equilibrium association constant Ka of 10⁺⁹ M⁻¹ or greater;
f. lack of cross-reactivity with other ILT family members;
g. cross-reactivity with cynomolgus ILT4; and/or
h. inhibiting tumor cells that express ILT4.

### F. Compositions

Also provided herein are compositions, e.g., a composition comprising one or a combination of any of the antibodies, or antigen binding fragments thereof, the bispecific constructs, or the multispecific constructs described herein, formulated together with a carrier (e.g., a pharmaceutically acceptable carrier).

As used herein, the terms “carrier” and “pharmaceutically acceptable carrier” includes any and all solvents, salts, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound (i.e., any of the antibodies, or antigen binding fragments thereof, the bispecific constructs, or the multispecific constructs described herein), may be coated in a material to protect the compound from the action of acids and other natural conditions that may inactivate the compound.

Examples of adjuvants which may be used with the antibodies, or antigen binding fragments thereof, the bispecific constructs, or the multispecific constructs described here include, but are not limited to: Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatised polysaccharides; polyphosphazenes; biodegradable microspheres; cytokines, such as GM-CSF, interleukin-2, -7, -12, and other like factors; 3D-MPL; CpG oligonucleotide; and monophosphoryl lipid A, for example 3-de-O-acylated monophosphoryl lipid A.

MPL adjuvants are available from Corixa Corporation (Seattle, Wash; see, for example, U.S. Pat. Nos. 4,436,727; 4,877,611; 4,866,034 and 4,912,094). CpG-containing oligonucleotides (in which the CpG dinucleotide is unmethylated) are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996.

Further alternative adjuvants include, for example, saponins, such as Quil A, or derivatives thereof, including QS21 and QS7 (Aquila Biopharmaceuticals Inc., Framingham, Mass.); Escin; Digitonin; or Gypsophila or Chenopodium quinoa saponins; Montanide ISA 720 (Seppic, France); SAF (Chiron, California, United States); ISCOMS (CSL), MF-59 (Chiron); the SBAS series of adjuvants (e.g., SBAS-2 or SBAS-4, available from SmithKline Beecham, Rixensart, Belgium); Detox (Enhanzyn™) (Corixa, Hamilton, Mont.); RC-529 (Corixa, Hamilton, Mont.) and other aminoalkyl glucosaminide 4-phosphates (AGPs); polyoxyethylene ether adjuvants such as those described in WO 99/52549A1; synthetic imidazoquinolines such as imiquimod [S-26308, R-837], (Harrison, et al., Vaccine 19: 1820-1826, 2001; and resiquimod [S-28463, R-848] (Vasilakos, et al., Cellular immunology 204: 64-74, 2000; Schiff bases of carbonyls and amines that are constitutively expressed on antigen presenting cell and T-cell surfaces, such as tucaresol (Rhodes, J. et al., Nature 377: 71-75, 1995); cytokine, chemokine and co-stimulatory molecules as either protein or peptide, including for example pro-inflammatory cytokines such as Interferon, GM-CSF, IL-1 alpha, IL-1 beta, TGF-alpha and TGF-beta, Th1 inducers such as interferon gamma, IL-2, IL-12, IL-15, IL-18 and IL-21, Th2 inducers such as IL-4, IL-5, IL-6, IL-10 and IL-13 and other chemokine and co-stimulatory genes such as MCP-1, MIP-1 alpha, MIP-1 beta, RANTES, TCA-3, CD80, CD86 and CD40L; immunostimulatory agents targeting ligands such as CTLA-4 and L-selectin, apoptosis stimulating proteins and peptides such as Fas; synthetic lipid based adjuvants, such as vaxfectin, (Reyes et al., Vaccine 19: 3778-3786, 2001) squalene, alpha-tocopherol, polysorbate 80, DOPC and cholesterol; endotoxin, [LPS], (Beutler, B., Current Opinion in Microbiology 3: 23-30, 2000); ligands that trigger Toll receptors to produce Th1-inducing cytokines, such as synthetic Mycobacterial lipoproteins, Mycobacterial protein p19, peptidoglycan, teichoic acid and lipid A; and CT (cholera toxin, subunits A and B) and LT (heat labile enterotoxin from E. coli, subunits A and B), heat shock protein family (HSPs), and LLO (listeriolysin 0; WO 01/72329). These and various further Toll-like Receptor (TLR) agonists are described for example in Kanzler et al, Nature Medicine, May 2007, Vol 13, No 5.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A composition of the present invention can be administered by a variety of methods known in the art. As will be appreciated by the skilled artisan, the route and/or mode of administration will vary depending upon the desired results. The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

To administer a compound of the invention by certain routes of administration, it may be necessary to coat the compound with, or co-administer the compound with, a material to prevent its inactivation. For example, the compound may be administered to a subject in an appropriate carrier, for example, liposomes, or a diluent. Acceptable diluents include saline and aqueous buffer solutions. Liposomes include water-in-oil-in-water CGF emulsions as well as conventional liposomes (Strejan et al. (1984) J. Neuroimmunol. 7:27).

Carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. For example, the antibodies of the invention may be administered once or twice weekly by subcutaneous or intramuscular injection or once or twice monthly by subcutaneous or intramuscular injection.

It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic compositions, formulations of the present invention include those suitable for oral, nasal, topical (including buccal and sublingual), rectal, vaginal and/or parenteral administration. The formulations may conveniently be presented in unit dosage form and may be prepared by any methods known in the art of pharmacy. The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.001 percent to about ninety percent of active ingredient, preferably from about 0.005 percent to about 70 percent, most preferably from about 0.01 percent to about 30 percent.

Formulations of the present invention which are suitable for vaginal administration also include pessaries, tampons, creams, gels, pastes, foams or spray formulations containing such carriers as are known in the art to be appropriate. Dosage forms for the topical or transdermal administration of compositions of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

When the compounds of the present invention are administered as pharmaceuticals, to humans and animals, they can be given alone or as a pharmaceutical composition containing, for example, 0.001 to 90% (more preferably, 0.005 to 70%, such as 0.01 to 30%) of active ingredient in combination with a pharmaceutically acceptable carrier.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of the present invention may be varied so as to obtain an amount of the active ingredient that is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. In general, a suitable daily dose of a composition of the invention will be that amount of the compound that is the lowest dose effective to produce a therapeutic effect. Such an effective dose will generally depend upon the factors described above. It is preferred that administration be intravenous, intramuscular, intraperitoneal, or subcutaneous, preferably administered proximal to the site of the target. If desired, the effective daily dose of a therapeutic composition may be administered as two, three, four, five, six or more sub-doses administered separately at appropriate intervals throughout the day, optionally, in unit dosage forms. While it is possible for a compound of the present invention to be administered alone, it is preferable to administer the compound as a pharmaceutical formulation (composition).

Therapeutic compositions can be administered with medical devices known in the art. For example, in a preferred embodiment, a therapeutic composition of the invention can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known to those skilled in the art.

In certain embodiments, the antibodies of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties that are selectively transported into specific cells or organs, thus enhance targeted drug delivery (see, e.g., V. V. Ranade (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134), different species of which may comprise the formulations of the inventions, as well as components of the invented molecules; p 120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273. In one embodiment of the invention, the therapeutic compounds of the invention are formulated in liposomes; in a more preferred embodiment, the liposomes include a targeting moiety. In a most preferred embodiment, the therapeutic compounds in the liposomes are delivered by bolus injection to a site proximal to the tumor or infection. The composition must be fluid to the extent that easy syringability exists. It must be stable under the conditions of manufacture and storage and must be preserved against the contaminating action of microorganisms such as bacteria and fungi.

The ability of a compound to inhibit cancer can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject. One of ordinary skill in the art would be able to determine such amounts based on such factors as the subject's size, the severity of the subject's symptoms, and the particular composition or route of administration selected.

The composition must be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound may be orally administered, for example, with an inert diluent or an assimilable edible carrier.

### G. Nucleic Acids

Also provided herein are isolated nucleic acid molecules encoding the antibodies, or antigen binding fragments thereof, bispecific constructs, and multispecific constructs, as well as expression vectors comprising such nucleic acids and host cells comprising such expression vectors. In one embodiment, a nucleic acid molecule coding for any of the antibodies, or antigen fragments thereof, bispecific constructs, or multispecific constructs described herein is provided. In another embodiment, the nucleic acid molecule is in the form of an expression vector. In another embodiment, the nucleic acid molecule is in the form of an expression vector which expresses the antibody, or antigen fragment thereof, bispecific construct, or the multispecific construct when administered to a subject in vivo.

In one embodiment, the nucleic acid molecule comprises a nucleotide sequence encoding an antibody variable region, wherein the antibody variable region comprises the amino acid sequence depicted in SEQ ID NO:9, 10, 19, 20, 97, 98, 99, 100, 101, 102, 103, 104, 105, 106, 107, 108, or an amino acid sequence at least 90% identical thereto (e.g., at least 90%, 91%, 92%, 93%, 94%, 95%, 96%, 97%, 98% or 99% identical to one or more of the aforementioned sequences).

In one embodiment, the nucleic acid molecule comprises a nucleotide sequence encoding an antibody chain, wherein the chain comprises the amino acid sequence depicted in SEQ ID NO:25, 26, 27, 28, or an amino acid sequence at least 90% identical thereto (e.g., at least 90%, 91%, 92%, 93%, 94%, 95%, 96%, 97%, 98% or 99% identical to one or more of the aforementioned sequences).

In another embodiment, the nucleic acid molecule comprises a nucleotide sequence encoding heavy and light chain variable regions of an antibody, wherein the heavy and light chain variable regions respectively comprise the amino acid sequences depicted in SEQ ID NOs:9 and 10, SEQ ID NOs:19 and 20, or amino acids sequences at least 90% identical to the aforementioned sequences (e.g., at least 90%, 91%, 92%, 93%, 94%, 95%, 96%, 97%, 98% or 99% identical).

In another embodiment, the nucleic acid molecule comprises a nucleotide sequence encoding heavy and light chains of an antibody, wherein the heavy and light chains respectively comprise the amino acid sequences depicted in SEQ ID NOs:25 and 26, SEQ ID NOs:27 and 28, or amino acids sequences at least 90% identical to the aforementioned sequences (e.g., at least 90%, 91%, 92%, 93%, 94%, 95%, 96%, 97%, 98% or 99% identical).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

### H. Combination Therapies

Any of the antibodies, antigen binding fragments thereof, bispecific constructs, and/or multispecific constructs described herein, can be administered in combination with an additional therapy, i.e., combined with other agents. The term “coadministered” as used herein includes any or all of simultaneous, separate, or sequential administration of the antibodies, antigen binding fragments thereof, bispecific constructs, or multispecific constructs described herein with adjuvants and other agents, including administration as part of a dosing regimen. For example, the combination therapy can include administering any of the antibodies, antigen binding fragments thereof, bispecific constructs, and/or multispecific constructs described herein with at least one or more additional therapeutic agents, such as anti-inflammatory agents, DMARDs (disease-modifying anti-rheumatic drugs), immunosuppressive agents, chemotherapeutics, radiation therapy, other antibodies, cytotoxins and/or drugs, as well as adjuvants, immunostimulatory agents and/or immunosuppressive agents.

Chemotherapeutic agents suitable for coadministration with the antibodies, antigen binding fragments thereof, bispecific constructs, and/or multispecific constructs described herein in the treatment of tumors include, for example: taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Further agents include, for example, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine) and temozolomide.

Agents that delete or inhibit immunosuppressive activities, for example, by immune cells (for example regulatory T-cells, NKT cells, macrophages, myeloid-derived suppressor cells, immature or suppressive dendritic cells) or suppressive factors produced by the tumor or host cells in the local microenvironment of the tumor (for example, TGFβ, indoleamine 2,3 dioxygenase—IDO), may also be administered with the binding domains, antibodies, antigen binding fragments thereof, bispecific constructs, and/or multispecific constructs described herein. Such agents include antibodies and small molecule drugs such as IDO inhibitors such as 1 methyl tryptophan or derivatives.

Suitable agents for coadministration with the antibodies, antigen binding fragments thereof, bispecific constructs, and/or multispecific constructs described herein for treatment of such immune disorders include for example, immunosuppressive agents such as rapamycin, cyclosporin and FK506; anti-TNF agents such as etanercept, adalimumab and infliximab; and steroids. Examples of specific natural and synthetic steroids include, for example: aldosterone, beclomethasone, betamethasone, budesonide, cloprednol, cortisone, cortivazol, deoxycortone, desonide, desoximetasone, dexamethasone, difluorocortolone, fluclorolone, flumethasone, flunisolide, fluocinolone, fluocinonide, fluocortin butyl, fluorocortisone, fluorocortolone, fluorometholone, flurandrenolone, fluticasone, halcinonide, hydrocortisone, icomethasone, meprednisone, methylprednisolone, paramethasone, prednisolone, prednisone, tixocortol and triamcinolone.

Suitable agents for coadministration with the antibodies, antigen binding fragments thereof, bispecific constructs, and/or multispecific constructs described herein for inducement or enhancement of an immune response include, for example, adjuvants and/or immunostimulatory agents, non-limiting examples of which have been disclosed hereinbefore. In one embodiment, the immunostimulatory agent is a TLR3 agonist, such as Poly IC.

As used herein, the term “immunostimulatory agent” includes, but is not limited to, compounds capable of stimulating antigen presenting cells (APCs), such as dendritic cells (DCs) and macrophages. For example, suitable immunostimulatory agents for use in the present invention are capable of stimulating APCs, so that the maturation process of the APCs is accelerated, the proliferation of APCs is increased, and/or the recruitment or release of co-stimulatory molecules (e.g., CD80, CD86, ICAM-1, MHC molecules and CCR7) and pro-inflammatory cytokines (e.g., IL-1β, IL-6, IL-12, IL-15, and IFN-γ) is upregulated. Suitable immunostimulatory agents are also capable of increasing T cell proliferation. Such immunostimulatory agents include, but are not be limited to, CD40 ligand; FLT 3 ligand; cytokines, such as IFN-α, IFN-β, IFN-γ and IL-2; colony-stimulating factors, such as G-CSF (granulocyte colony-stimulating factor) and GM-CSF (granulocyte-macrophage colony-stimulating factor); an CTLA-4 antibody, PD-1 antibody, 41BB antibody, or OX-40 antibody; LPS (endotoxin); ssRNA; dsRNA; Bacille Calmette-Guerin (BCG); Levamisole hydrochloride; and intravenous immune globulins. In one embodiment an immunostimulatory agent may be a Toll-like Receptor (TLR) agonist. For example the immunostimulatory agent may be a TLR3 agonist such as double-stranded inosine:cytosine polynucleotide (Poly I:C, for example available as Ampligen™ from Hemispherx Bipharma, PA, US or Poly IC:LC from Oncovir) or Poly A:U; a TLR4 agonist such as monophosphoryl lipid A (MPL) or RC-529 (for example as available from GSK, UK); a TLR5 agonist such as flagellin; a TLR7 or TLR8 agonist such as an imidazoquinoline TLR7 or TLR 8 agonist, for example imiquimod (eg Aldara™) or resiquimod and related imidazoquinoline agents (for example as available from 3M Corporation); or a TLR 9 agonist such as a deoxynucleotide with unmethylated CpG motifs (so-called “CpGs”, for example as available from Coley Pharmaceutical). Such immunostimulatory agents may be administered simultaneously, separately or sequentially with the antibodies, antigen binding fragments thereof, bispecific constructs, and/or multispecific constructs described herein.

### I. USES AND METHODS OF THE INVENTION

Also provided herein are methods of methods of inducing or enhancing an immune response, and methods of treating cancer by administering the bispecific constructs, multispecific constructs, antibodies, or antigen binding fragments thereof, or compositions described herein to a patient in need thereof.

The terms “inducing an immune response” and “enhancing an immune response” are used interchangeably and refer the stimulation of an immune response (i.e., either passive or adaptive) to a particular antigen.

The terms “treat,” “treating,” and “treatment,” as used herein, refer to therapeutic or preventative measures described herein. The methods of “treatment” employ administration to a subject, in need of such treatment, a bispecific construct, multispecific construct, antibody, antigen binding fragment thereof, or composition as described herein, for example, a subject in need of an enhanced immune response against a particular antigen or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

The term “effective dose” or “effective dosage” is defined as an amount sufficient to achieve or at least partially achieve the desired effect. The term “therapeutically effective dose” is defined as an amount sufficient to cure or at least partially arrest the disease and its complications in a patient already suffering from the disease. Amounts effective for this use will depend upon the severity of the disorder being treated and the general state of the patient's own immune system.

The term “patient” includes human and other mammalian subjects that receive either prophylactic or therapeutic treatment.

As used herein, the term “inhibits growth” (e.g., referring to cells) is intended to include any measurable decrease in the growth of a cell, e.g., the inhibition of growth of a cell by at least about 10%, 20%, 30%, 40%, 50%, 60%, 70%, 80%, 90%, 99%, or 100%.

In another aspect, methods for inducing or enhancing an immune response (e.g., against an antigen) in a subject comprising administering to the subject any one of the antibodies, or antigen binding fragments thereof, bispecific constructs, multispecific constructs, or the compositions described herein, in an amount effective to induce or enhance an immune response in the subject (e.g., against an antigen).

In another aspect, methods of for treating cancer in a subject are provided, the method comprising administering to the subject any one of the antibodies, or antigen binding fragments thereof, bispecific constructs, multispecific constructs, or the compositions described herein, in an amount effective to treat the condition or disease.

In another aspect, methods for treating cancer in a subject are provided, wherein the method comprises administering to the subject any one of the ILT4 antibodies, or antigen binding fragments thereof, described herein in combination with another antibody or antigen binding fragment thereof, e.g., any one of the PD-L1 or PD-1 antibodies, or antigen binding fragments thereof, described herein.

In one embodiment, the ILT4 antibody, or antigen binding fragment thereof, and the PD-L1 or PD-1 antibody, or antigen binding fragment thereof, are administered separately. In one embodiment, the ILT4 antibody, or antigen binding fragment thereof, and the PD-L1 or PD-1 antibody, or antigen binding fragment thereof, are administered sequentially. For example, the ILT4 antibody, or antigen binding fragment thereof, can be administered first followed by (e.g., immediately followed by) administration of the PD-L1 or PD-1 antibody, or antigen binding fragment thereof, or vice versa. In another embodiment, the ILT4 antibody, or antigen binding fragment thereof, and the PD-L1 or PD-1 antibody, or antigen binding fragment thereof, are administered together. In another embodiment, the ILT4 antibody, or antigen binding fragment thereof, and the PD-L1 or PD-1 antibody, or antigen binding fragment thereof, are administered simultaneously. In another embodiment, the ILT4 antibody, or antigen binding fragment thereof, and the PD-L1 or PD-1 antibody, or antigen binding fragment thereof, are simultaneously administered in a single formulation. Alternatively, the ILT4 antibody, or antigen binding fragment thereof, and the PD-L1 or PD-1 antibody, or antigen binding fragment thereof, are formulated for separate administration and are administered concurrently or sequentially. Such concurrent or sequential administration preferably results in both antibodies being simultaneously present in treated patients.

In certain embodiments, administration of any of the ILT4 antibodies, or antigen binding fragment thereof, described herein in combination with any of the PD-L1 or PD-1 antibodies, or antigen binding fragments thereof, described herein results in synergistic effects (e.g., in enhancing immune responses in vivo) as compared to use of either antibody alone.

The subject can be, for example, one who suffers from a condition or disease in which stimulation of an immune response is desired. In one embodiment, the condition or disease is cancer. Types of cancers include, but are not limited to, leukemia, acute lymphocytic leukemia, acute myelocytic leukemia, myeloblasts promyelocyte myelomonocytic monocytic erythroleukemia, chronic leukemia, chronic myelocytic (granulocytic) leukemia, chronic lymphocytic leukemia, mantle cell lymphoma, primary central nervous system lymphoma, Burkitt's lymphoma and marginal zone B cell lymphoma, Polycythemia vera Lymphoma, Hodgkin's disease, non-Hodgkin's disease, multiple myeloma, Waldenstrom's macroglobulinemia, heavy chain disease, solid tumors, sarcomas, and carcinomas, fibrosarcoma, myxosarcoma, liposarcoma, chrondrosarcoma, osteogenic sarcoma, osteosarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon sarcoma, colorectal carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular tumor, lung carcinoma, small cell lung carcinoma, non small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, retinoblastoma, nasopharyngeal carcinoma, esophageal carcinoma, basal cell carcinoma, biliary tract cancer, bladder cancer, bone cancer, brain and central nervous system (CNS) cancer, cervical cancer, choriocarcinoma, colorectal cancers, connective tissue cancer, cancer of the digestive system, endometrial cancer, esophageal cancer, eye cancer, head and neck cancer, gastric cancer, intraepithelial neoplasm, kidney cancer, larynx cancer, liver cancer, lung cancer (small cell, large cell), melanoma, neuroblastoma; oral cavity cancer (for example lip, tongue, mouth and pharynx), ovarian cancer, pancreatic cancer, retinoblastoma, rhabdomyosarcoma, rectal cancer; cancer of the respiratory system, sarcoma, skin cancer, stomach cancer, testicular cancer, thyroid cancer, uterine cancer, and cancer of the urinary system. Particular cancers include ILT4-expressing tumors selected from the group consisting of chronic lymphocytic leukemia, mantle cell lymphoma, primary central nervous system lymphoma, Burkitt's lymphoma and marginal zone B cell lymphoma. Other disease indications include bacterial, fungal, viral and parasitic infectious diseases.

The methods of inducing or enhancing an immune response (e.g., against an antigen) in a subject described herein can further comprise administering the antigen to the subject. As used herein, the term “antigen” refers to any natural or synthetic immunogenic substance, such as a protein, peptide, hapten, polysaccharide and/or lipid. The bispecific construct, multispecific construct, antibody, antigen binding fragment thereof, or composition described herein and antigen can be administered at the same time or, alternatively, the bispecific construct, multispecific construct, antibody, antigen binding fragment thereof, or composition can be administered before or after the antigen is administered.

In one embodiment, a bispecific construct, multispecific construct, antibody, antigen binding fragment thereof, or composition described herein is administered in combination with a vaccine, to enhance the immune response against the vaccine antigen, for example a tumor antigen (to thereby enhance the immune response against the tumor) or an antigen from an infectious disease pathogen (to thereby enhance the immune response against the infectious disease pathogen). Accordingly, in one embodiment, a vaccine antigen can comprise, for example, an antigen or antigenic composition capable of eliciting an immune response against a tumor or against an infectious disease pathogen such as a virus, a bacteria, a parasite or a fungus. The antigen or antigens be derived from tumors, such as the various tumor antigens previously disclosed herein. Alternatively, the antigen or antigens can be derived from pathogens such as viruses, bacteria, parasites and/or fungi.

Preferred antigens to be co-administered with the antibodies, or antigen binding fragments thereof, bispecific constructs, multispecific constructs, or the compositions of described herein include tumor antigens and vaccine antigens (e.g., bacterial, viral or other pathogen antigens against which protective immunity is desired to be raised in a subject for purposes of vaccination). Additional examples of suitable pathogen antigens include tumor-associated antigens (TAAs), including but not limited to, sequences comprising all or part of the sequences of EGFR, EGFRvIII, gp100 or Pmel17, HER2/neu, mesothelin, CEA, MART1, MAGE-A1, MAGE-A2, MAGE-A3, MAGE-A4, MUC-1, GPNMB, HMW-MAA, TIM1, ROR1, CD19 and germ cell derived tumor antigens.

Other suitable antigens include viral antigens for the prevention or treatment of viral diseases. Examples of viral antigens include, but are not limited to, HIV-1 env, HBsAg, HPV, FAS, HSV-1, HSV-2, p17, ORF2 and ORF3 antigens. In addition, viral antigens or antigenic determinants can be derived from, for example: Cytomegalovirus (especially Human, such as gB or derivatives thereof); Epstein Barr virus (such as gp350); flaviviruses (e.g. Yellow Fever Virus, Dengue Virus, Tick-borne encephalitis virus, Japanese Encephalitis Virus); hepatitis virus such as hepatitis B virus (for example Hepatitis B Surface antigen such as the PreS1, PreS2 and S antigens described in EP-A-414 374; EP-A-0304 578, and EP-A-198474), hepatitis A virus, hepatitis C virus and hepatitis E virus; HIV-1, (such as tat, nef, gp120 or gp160); human herpes viruses, such as gD or derivatives thereof or Immediate Early protein such as ICP27 from HSV1 or HSV2; human papilloma viruses (for example HPV6, 11, 16, 18); Influenza virus (whole live or inactivated virus, split influenza virus, grown in eggs or MDCK cells, or Vero cells or whole flu virosomes (as described by Gluck, Vaccine, 1992,10, 915-920) or purified or recombinant proteins thereof, such as NP, NA, HA, or M proteins); measles virus; mumps virus; parainfluenza virus; rabies virus; Respiratory Syncytial virus (such as F and G proteins); rotavirus (including live attenuated viruses); smallpox virus; Varicella Zoster Virus (such as gpI, II and IE63); and the HPV viruses responsible for cervical cancer (for example the early proteins E6 or E7 in fusion with a protein D carrier to form Protein D-E6 or E7 fusions from HPV 16, or combinations thereof; or combinations of E6 or E7 with L2 (see for example WO 96/26277).

Examples of bacterial antigens include, but are not limited to, Toxoplasma gondii or Treponema pallidum. The bacterial antigens can be in the treatment or prevention of various bacterial diseases such as Anthrax, Botulism, Tetanus, Chlamydia, Cholera, Diphtheria, Lyme Disease, Syphilis and Tuberculosis. Bacterial antigens or antigenic determinants can be derived from, for example: Bacillus spp., including B. anthracis (e.g., botulinum toxin); Bordetella spp, including B. pertussis (for example pertactin, pertussis toxin, filamenteous hemagglutinin, adenylate cyclase, fimbriae); Borrelia spp., including B. burgdorferi (eg OspA, OspC, DbpA, DbpB), B. garinii (eg OspA, OspC, DbpA, DbpB), B. afzelii (eg OspA, OspC, DbpA, DbpB), B. andersonii (eg OspA, OspC, DbpA, DbpB), B. hermsii; Campylobacter spp, including C. jejuni (for example toxins, adhesins and invasins) and C. coli; Chlamydia spp., including C. trachomatis (eg MOMP, heparin-binding proteins), C. pneumonie (eg MOMP, heparin-binding proteins), C. psittaci; Clostridium spp., including C. tetani (such as tetanus toxin), C. botulinum (for example botulinum toxin), C. difficile (eg clostridium toxins A or B); Corynebacterium spp., including C. diphtheriae (eg diphtheria toxin); Ehrlichia spp., including E. equi and the agent of the Human Granulocytic Ehrlichiosis; Rickettsia spp, including R. rickettsii; Enterococcus spp., including E. faecalis, E. faecium; Escherichia spp, including enterotoxic E. coli (for example colonization factors, heat-labile toxin or derivatives thereof, or heat-stable toxin), enterohemorragic E. coli, enteropathogenic E. coli (for example shiga toxin-like toxin); Haemophilus spp., including H. influenzae type B (eg PRP), non-typable H. influenzae, for example OMP26, high molecular weight adhesins, P5, P6, protein D and lipoprotein D, and fimbrin and fimbrin derived peptides (see for example U.S. Pat. No. 5,843,464); Helicobacter spp, including H. pylori (for example urease, catalase, vacuolating toxin); Pseudomonas spp, including P. aeruginosa; Legionella spp, including L. pneumophila; Leptospira spp., including L. interrogans; Listeria spp., including L. monocytogenes; Moraxella spp, including M catarrhalis, also known as Branhamella catarrhalis (for example high and low molecular weight adhesins and invasins); Morexella Catarrhalis (including outer membrane vesicles thereof, and OMP106 (see for example WO97/41731)); Mycobacterium spp., including M. tuberculosis (for example ESAT6, Antigen 85A, —B or —C), M. bovis, M. leprae, M. avium, M. paratuberculosis, M. smegmatis; Neisseria spp, including N. gonorrhea and N. meningitidis (for example capsular polysaccharides and conjugates thereof, transferrin-binding proteins, lactoferrin binding proteins, PilC, adhesins); Neisseria mengitidis B (including outer membrane vesicles thereof, and NspA (see for example WO 96/29412); Salmonella spp, including S. typhi, S. paratyphi, S. choleraesuis, S. enteritidis; Shigella spp, including S. sonnei, S. dysenteriae, S. flexnerii; Staphylococcus spp., including S. aureus, S. epidermidis; Streptococcus spp, including S. pneumonie (e.g., capsular polysaccharides and conjugates thereof, PsaA, PspA, streptolysin, choline-binding proteins) and the protein antigen Pneumolysin (Biochem Biophys Acta, 1989,67,1007; Rubins et al., Microbial Pathogenesis, 25,337-342), and mutant detoxified derivatives thereof (see for example WO 90/06951; WO 99/03884); Treponema spp., including T. pallidum (eg the outer membrane proteins), T. denticola, T. hyodysenteriae; Vibrio spp, including V. cholera (for example cholera toxin); and Yersinia spp, including Y. enterocolitica (for example a Yop protein), Y. pestis, Y. pseudotuberculosis.

Parasitic/fungal antigens or antigenic determinants can be derived from, for example: Babesia spp., including B. microti; Candida spp., including C. albicans; Cryptococcus spp., including C. neoformans; Entamoeba spp., including E. histolytica; Giardia spp., including; G. lamblia; Leshmania spp., including L. major; Plasmodium. faciparum (MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.); Pneumocystis spp., including P. carinii; Schisostoma spp., including S. mansoni; Trichomonas spp., including T. vaginalis; Toxoplasma spp., including T. gondii (for example SAG2, SAG3, Tg34); Trypanosoma spp., including T. cruzi.

It will be appreciated that in accordance with this aspect of the present invention antigens and antigenic determinants can be used in many different forms. For example, antigens or antigenic determinants can be present as isolated proteins or peptides (for example in so-called “subunit vaccines”) or, for example, as cell-associated or virus-associated antigens or antigenic determinants (for example in either live or killed pathogen strains). Live pathogens will preferably be attenuated in known manner. Alternatively, antigens or antigenic determinants may be generated in situ in the subject by use of a polynucleotide coding for an antigen or antigenic determinant (as in so-called “DNA vaccination”), although it will be appreciated that the polynucleotides which can be used with this approach are not limited to DNA, and may also include RNA and modified polynucleotides as discussed above.

In one embodiment, a vaccine antigen can also be targeted, for example to particular cell types or to particular tissues. For example, the vaccine antigen can be targeted to Antigen Presenting Cells (APCs), for example by use of agents such as antibodies targeted to APC-surface receptors such as DEC-205, for example as discussed in WO 2009/061996 (Celldex Therapeutics, Inc), or the Mannose Receptor (CD206) for example as discussed in WO 03040169 (Medarex, Inc.).

### J. Kits

Also provided are kits (e.g., diagnostic kits) comprising one or more ILT4 antibody or antigen binding fragment thereof, bispecific constructs, multispecific constructs, or compositions as described herein, optionally with instructions for use. Kits may also include informative pamphlets, for example, pamphlets informing one how to use reagents to practice a method disclosed herein. The term “pamphlet” includes any writing, marketing materials or recorded material supplied on or with the kit, or which otherwise accompanies the kit.

The present invention is further illustrated by the following examples, which should not be construed as further limiting. The contents of figures and all references, patents and published patent applications cited throughout this application are expressly incorporated herein by reference.

### V. EXAMPLES

### Example 1: Generation of Murine Antibodies

Murine ILT4 monoclonal antibodies were generated by immunizing BALB/c mice with a soluble human ILT4 antigen. The antigen used was a soluble fusion protein comprising an ILT4 extracellular domain with a HIS tag (R&D Systems® or AcroBiosystems®). The antigen (i.e., 5-20 micrograms soluble recombinant ILT4 antigen in PBS) was mixed at 1:1 ratio with MPL plus TDM adjuvant system (Sigma®). Mice were injected with 200 microliters of the prepared antigen into the peritoneal cavity approximately every 14 days. Animals that developed anti-ILT4 titers were given an intravenous injection of 1-10 micrograms soluble recombinant ILT4 antigen three to four days prior to fusion. Mouse spleens were harvested, and the isolated splenocytes used for hybridoma preparation.

The P3x63Ag8.653 murine myeloma cell line (ATCC CRL 1580) was used for fusions which was cultured in RPMI 1640 (Invitrogen®) containing 10% FBS. Additional media supplements were added to the hybridoma growth media, which included: up to 10% Hybridoma Cloning Supplement (Sigma), 10% FBS (Sigma), L-glutamine (Gibco®) 0.1% gentamycin (Gibco), 2-mercaptoethanol (Gibco), with HAT (Sigma; 1.0×10⁴ M hypoxanthine, 4.0×10⁻⁷ M aminopterin, 1.6×10⁻⁵ M thymidine media.

Spleen cells were mixed with the P3×63Ag8.653 myeloma cells in a 6:1 ratio and pelleted by centrifugation. Polyethylene glycol was added dropwise with careful mixing to facilitate fusion. Hybridomas were cultured for two to three weeks until visible colonies become established. Supernatant was harvested and used for initial screening for mouse IgG via ELISA using a human soluble ILT4 fusion protein and a mouse Fc specific detection. IgG positive supernatants were then assayed for ILT4 specificity via flow cytometry. The hybridomas were also screened for cross-reactivity with cynomolgus macaque ILT4 and all were positive for binding.

Hybridoma cells were expanded and cell pellets were frozen for RNA isolation and sequencing. The V_{H} and V_{L} coding regions of human monoclonal antibodies were identified using RNA from the corresponding hybridomas. RNA was reverse transcribed to cDNA, the V coding regions were amplified by PCR and the PCR product was sequenced, inserted into human IgG4 vector, transiently expressed as IgG4 chimeric antibodies and purified by protein A column chromatography which led to the isolation of two antibodies of particular interest, the variable region sequences of which were designated 7A3 (SEQ ID NOs: 9 and 10) and 7B1 (SEQ ID NOs: 19 and 20)).

### Example 2: Generation of Humanized Antibodies

A computer model of the parental heavy and light chain variable region domains (i.e., VH and VL domains) of antibodies 7A3 and 7B1 from Example 1 was produced and used to guide the humanization process. The original mouse variable sequences of antibodies 7A3 and 7B1 were aligned to all human germline sequences. The original mouse and closest matching germline sequences were analyzed for sequence liabilities and the most appropriate germline frameworks selected. Complementarity determining regions (CDRs) from the parent antibody were grafted onto an appropriate number of human frameworks and back mutations were introduced as necessary.

Four heavy chain and four light chain humanized variants were designed for antibody 7A3. The 7A3 heavy chain variants were designated: 7A3-H1 (SEQ ID NO:97), 7A3-H2 (SEQ ID NO:9), 7A3-H3 (SEQ ID NO:98) and 7A3-H4 (SEQ ID NO: 99). The 7A3 light chain variants were designated: 7A3-L1 (SEQ ID NO:10), 7A3-L2 (SEQ ID NO:100), 7A3-L3 (SEQ ID NO:101) and 7A3-L4 (SEQ ID NO:102).

Four heavy chain and four light chain humanized variants were designed for antibody 7B1. The 7B1 heavy chain variants were designated: 7B1-H1 (SEQ ID NO:103), 7B1-H2 (SEQ ID NO:19), 7B1-H3 (SEQ ID NO:104) and 7B1-H4 (SEQ ID NO:105). The 7B1 light chain variants were designated: 7B1-L1 (SEQ ID NO:20), 7B1-L2 (SEQ ID NO:106), 7B1-L3 (SEQ ID NO:107) and 7B1-L4 (SEQ ID NO:108). Pairing of these variable domain sequences is shown in Table 4. Antibodies 7A3 VH6-L17 and 7B1 VH10-L21 were protein A purified, their Fc domains were mutated (AQQ), and selected for further investigation as described below.

TABLE 4
Heavy and Light Chain Pairings

| Heavy Chain | Light Chain | Resulting Antibody |
|---|---|---|
| 7A3-H2 | 7A3-L1 | 7A3 VH6-L17 |
| 7A3-H2 | 7A3-L2 | 7A3 VH6-L18 |
| 7A3-H2 | 7A3-L4 | 7A3 VH6-L20 |
| 7B1-H2 | 7B1-L1 | 7B1 VH10-L21 |
| 7B1-H2 | 7B1-L2 | 7B1 VH10-L22 |
| 7B1-H2 | 7B1-L4 | 7B1 VH10-L24 |

### Example 3: Determination of Affinity and Rate Constants of Humanized Monoclonal Antibodies by Bio-Layer Interferometry (BLI)

Binding affinity and binding kinetics of various humanized ILT4 antibodies were examined by bio-layer interferometry (BLI) using an Octet™ QK^{e} instrument (ForteBio Sartorius®, Fremont, CA) according to the manufacturer's guidelines.

Purified chimeric antibodies (7A3-huG4 and 7B1-huG4) and humanized antibodies (7A3 VH6-L17, 7A3 VH6-L18, 7A3 VH6-L20, 7B1 VH10-L21, 7B1 VH10-L22 and 7B1 VH10-L24) were captured on Anti-Human Fc Capture (AHC) biosensors (Fortebio Product No. 18-5060). Each antibody was prepared in dilution buffer (10mMPO4+150 mM NaCl+1 mg/mL BSA+0.05% Tween 20, pH 7.2) to 0.5 μg/mL and loaded on freshly hydrated and pre-conditioned AHC biosensors for 300 seconds at 30° C. and 1000 rpm plate shake speed. For one assay, eight biosensors were loaded with the same antibody.

Binding was determined by exposing seven of the antibody loaded biosensors to analyte: soluble human ILT4-HIS (His-tagged ILT4 extracellular domain). Affinity measurements were determined using 2-fold serial dilutions of analyte ranging from 25 to 0.4 nM in dilution buffer at 30° C. and 1000 rpm plate shake speed. Association of the antibody loaded biosensors in analyte wells was carried out for 300 seconds, the biosensors were then moved to dilution buffer wells for 1500 seconds for dissociation measurements.

Corresponding controls were conducted in each case by keeping the remaining biosensor with captured antibody in dilution buffer well for association and dissociation steps. The data for the control biosensor was used to subtract background and account for biosensor drift and antibody dissociation from the biosensors.

Fortebio's Data Analysis Software version 10.0.3.1 (ForteBio Sartorius, Fremont, CA) was used in each case to derive kinetic parameters from the concentration series of analyte in dilution buffer binding to captured antibody. The association and dissociation curves were fitted to a 1:1 binding model using the data analysis software according to the manufacturer's guidelines.

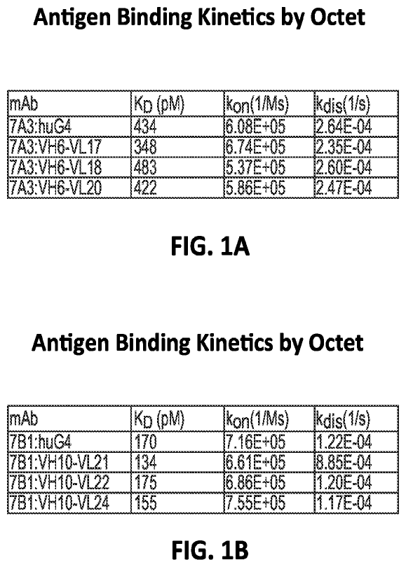

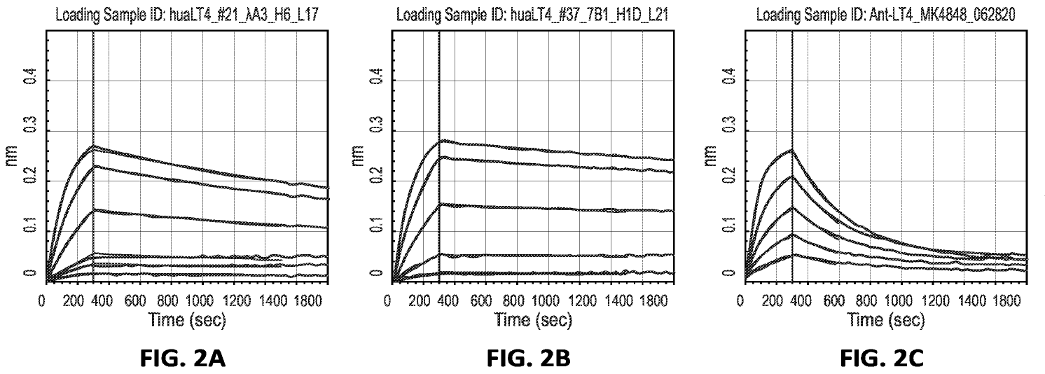

The affinity and kinetic parameters (with background subtracted) as determined are shown in FIGS. 1A and 1B, where kₒₙ=rate of association, k_{dis}=rate of dissociation, and K_{D}=affinity constant, determined by the ratio k_{dis}/kₒₙ. Representative traces are shown in FIG. 2.

### Example 4: Binding of Chimeric and Humanized Monoclonal Antibodies to Human ILT4 Using ELISA

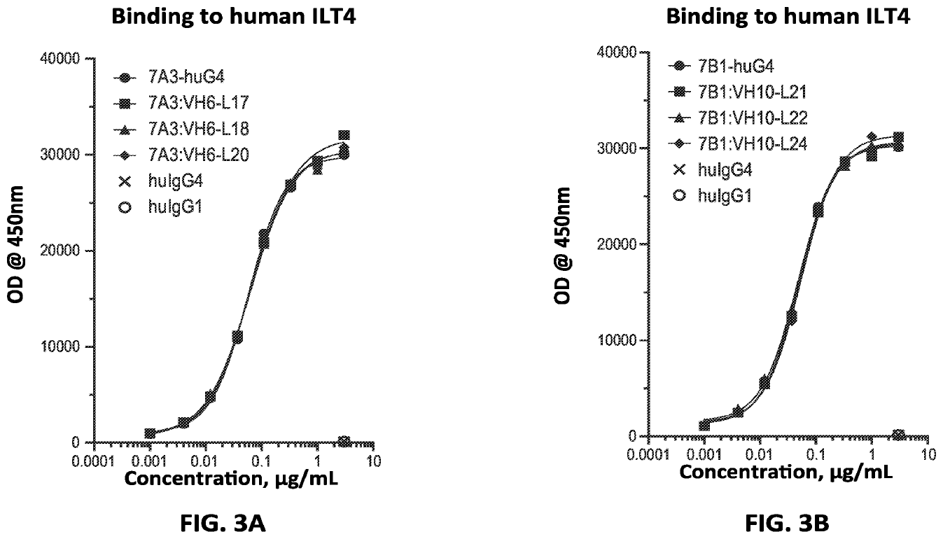

Microtiter plates were coated with recombinant human ILT4-kappa in PBS, and then blocked with 5% bovine serum albumin in PBS. Protein A purified chimeric monoclonal antibodies (7A3-huG4, 7B1-huG4), several of their humanized versions (7A3 VH6-L17, 7A3 VH6-L18, 7A3 VH6-L20, 7B1 VH10-L21, 7B1 VH10-L22 and 7B1 VH10-L24) and isotype controls were added at various concentrations and incubated at 37° C. The plates were washed with PBS/Tween and then incubated with a goat-anti-human IgG Fc-specific polyclonal reagent conjugated to horseradish peroxidase at 37° C. After washing, the plates were developed with HRP substrate, and analyzed at OD 450 using a microtiter plate reader. Representative binding curves are shown in FIGS. 3A and 3B.

### Example 5: Binding of Chimeric and Humanized Monoclonal Antibodies to Cells Expressing Human ILT4

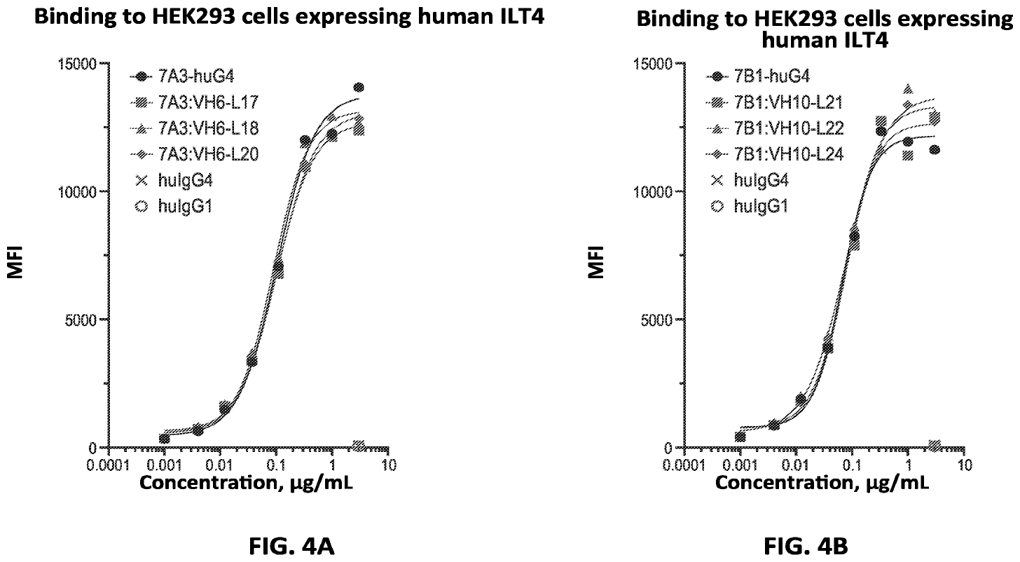

Antibodies were tested for binding to human HEK293 cell lines expressing human ILT4 on their surface. Protein A purified chimeric monoclonal antibodies (7A3-huG4, 7B1-huG4), various humanized versions (7A3 VH6-L17, 7A3 VH6-L18, 7A3 VH6-L20, 7B1 VH10-L21, 7B1 VH10-L22 and 7B1 VH10-L24) and isotype controls were incubated with HEK293 cells expressing human ILT4 at room temperature on a plate shaker. After 20 minutes, the cells were washed with PBS containing 0.1% BSA and 0.05% NaN₃ (PBA) and the bound antibodies were detected by incubating the cells with a PE labeled goat anti-human IgG Fc-specific probe. The excess probe was washed from the cells with PBA and the cell associated fluorescence was determined by analysis using a FACSCanto II™ instrument (BD Biosciences, NJ, USA) according to the manufacturer's directions. Representatives binding curves are shown in FIGS. 4A and 4B.

### Example 6: Induction of TNF-α Production by Chimeric and Humanized Monoclonal Antibodies in Macrophages

Macrophages were derived from human monocytes as follows: PBMCs were added to T175 cm² flasks and monocytes allowed to adhere for approximately 2 hours at 37° C., 6% CO₂. The non-adherent cells were removed and the monocytes cultured for 7 days in RPMI containing 10% FBS and 50 ng/mL M-CSF (R&D Systems®).

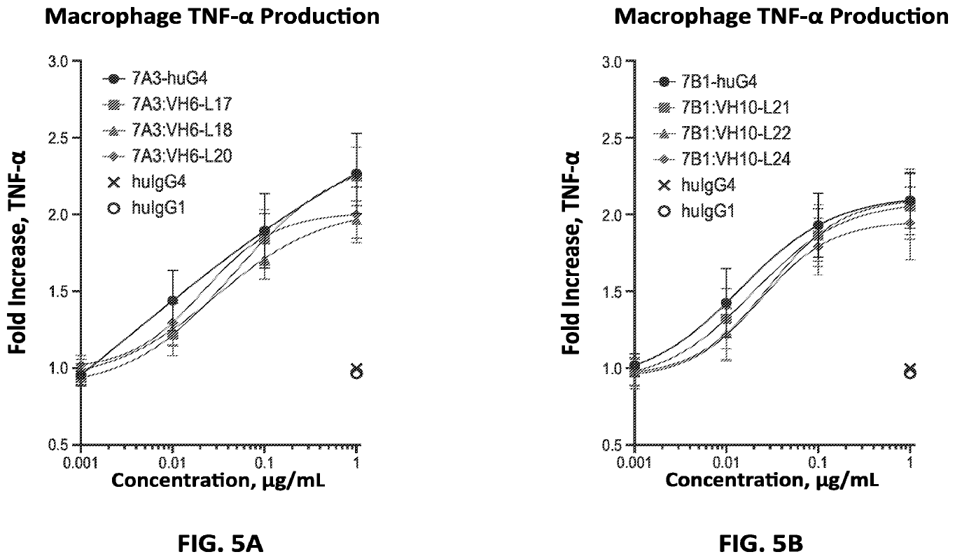

The cells were then incubated in the presence of protein A purified chimeric monoclonal antibodies (7A3-huG4, 7B1-huG4), their humanized versions (7A3 VH6-L17, 7A3 VH6-L18, 7A3 VH6-L20, 7B1 VH10-L21, 7B1 VH10-L22 and 7B1 VH10-L24) and isotype controls and of 50 ng/mL LPS (Invivogen) at 37° C., 6% CO₂. After 24 hours, the cells were harvested and the supernatant was collected and stored for cytokine analysis. Induction of TNF-α was evaluated in the supernatants collected by ELISA (R&D Systems). FIGS. 5A and 5B show the increase in TNF-α production with the various ILT4 antibodies.

### Example 7: Induction of TNF-α and MIP1-γ Production by Humanized Monoclonal Antibodies in Macrophages

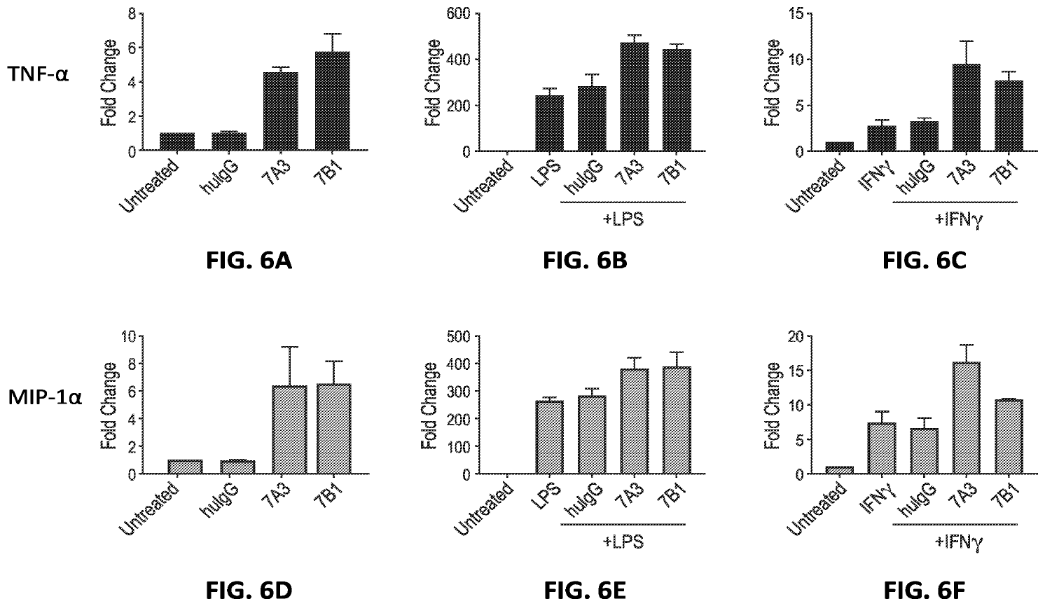

Human PBMCs were differentiated with MCSF (100 ng/mL) for 7 days. Following differentiation, human macrophages were plated at 1.5×10⁶ cells/well and allowed to adhere overnight. The following day, media was removed, and cells were treated with monoclonal antibody (100 nM) with or without LPS (10 ng/mL), or IFNγ (10 ng/mL) for 24 hrs. Following treatment, conditioned supernatants were removed and stored at −80° C. until ready to run ELISAs for human TNF-α and MIP1-α (R&D Systems) following manufacturer's protocols. Experiments were performed in triplicate. Results are shown in FIGS. 6A-6F.

### Example 8: Gene Expression Analysis of Humanized Monoclonal Antibodies in Macrophages

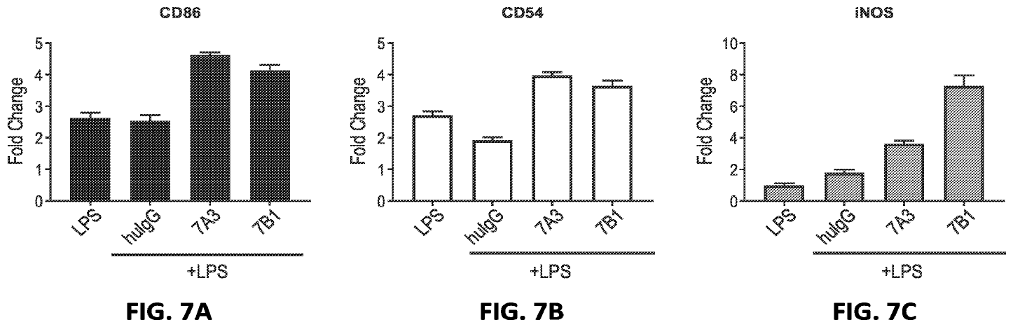

Human PBMCs were differentiated with MCSF (100 ng/mL) for 7 days. After differentiation human macrophages were plated at 1.5×10⁶ cells/well and allowed to adhere overnight. The following day, media was removed, and cells were treated with monoclonal antibody (100 nM) in the presence of LPS (10 ng/mL) for 24 hrs. Following treatment, cells were lysed with RLT buffer and RNA extracted using RNEasy MiniKit Plus (Qiagen®) following manufacturer's protocol. cDNA synthesis was performed using Superscript IV VILO Mastermix following manufacturer's protocols with 1 μg of input total RNA. Gene expression was measured (HPRT, CD86, iNOS, CD54) by Quantitative Real Time PCR using SYBR Green Mastermix (Applied Biosystems®) and plates run on the 7900HT Fast Real-Time PCR System (ThermoFisher®). Relative gene expression was measured using the 2-ΔΔCt method and HPRT as a house-keeping gene. Experiments were performed in duplicate. Results are shown in FIGS. 7A, 7B, and 7C.

### Example 9: Cross-Reactivity of Humanized Monoclonal Antibodies: Binding to Cells Expressing ILT Family Members

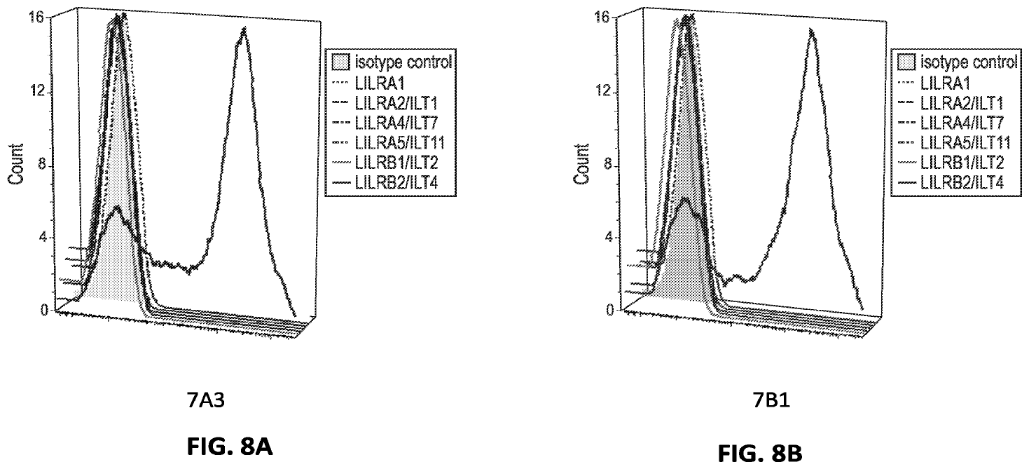

Protein A purified humanized monoclonal antibodies 7A3 VH6-L17 and 7B1 VH10-L21 and isotype controls were incubated with CHO cells expressing human ILT family members with highest homology to ILT4, e.g., LILRA1, LILRA2 (ILT1), LILRA4 (ILT7), LILRA5 (ILT11), LILRB1 (ILT2) and LILRB2 at room temperature on a plate shaker. After 20 minutes, the cells were washed with PBS containing 0.1% BSA and 0.05% NaN₃ (PBA) and the bound antibodies were detected by incubating the cells with a PE labeled goat anti-human IgG Fc-specific probe. The excess probe was washed from the cells with PBA and the cell associated fluorescence was determined by analysis using a FACSCanto II™ instrument (BD Biosciences®, NJ, USA) according to the manufacturer's directions. Representative binding is shown in FIGS. 8A and 8B.

### Example 10: Binding of Humanized Monoclonal Antibodies to Myeloid Cells

Macrophages were prepared as described previously in Example 6. Dendritic cells were prepared as follows: PBMC's were added to T175 cm² flasks and monocytes allowed to adhere for approximately 2 hours at 37° C., 6% CO₂. The non-adherent cells were removed and the monocytes cultured for 7 days in RPMI containing 10% FBS, 100 ng/mL GM-CSF (R&D Systems) and 10 ng/mL IL-4 (R&D Systems).

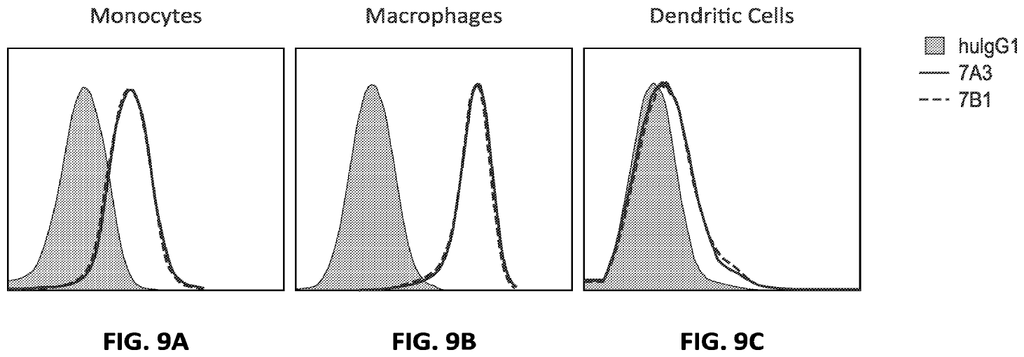

Protein A purified monoclonal antibodies 7A3 VH6-L17 and 7B1 VH10-L21 and isotype controls were incubated with human monocytes, macrophages and dendritic cells at room temperature on a plate shaker. After 20 minutes, the cells were washed with PBS containing 0.1% BSA and 0.05% NaN₃ (PBA) and the bound antibodies were detected by incubating the cells with a PE labeled goat anti-human IgG Fc-specific probe. The excess probe was washed from the cells with PBA and the cell associated fluorescence was determined by analysis using a FACSCanto II™ instrument (BD Biosciences, NJ, USA) according to the manufacturer's directions. Representative binding is shown in FIGS. 9A, 9B, and 9C.

### Example 11: Construction and Production of Bispecific Antibodies

Tetravalent bispecific antibody constructs were developed using a mutated fully human IgG1 backbone for a PD-L1 monoclonal antibody sequence (9H9; see SEQ ID NOs: 87 and 88 of WO2019204462 for heavy and light chain sequences) and the scFv of the ILT4 monoclonal antibody genetically linked to the C-terminus of the 9H9 heavy chain through a linker. Such bispecific antibodies were created with scFv versions of both 7A3 and 7B1 antibodies. The humanized antibody scFv sequences used in the bispecifics were taken from 7A3 VH6-L17 and 7B1 VH10-L21, respectively. The Fc domain was mutated (AQQ) and certain other amino acid residues were also modified. These constructs are denoted 9H9-7A3 HL and 9H9-7B1 HL, respectively, in which the scFv is in the VH-VL orientation.

Alternative bispecific antibody constructs were created with the heavy and light chains of the anti-ILT4 scFv in the reverse orientation, in which the scFv is in the VL-VH orientation. These constructs are denoted 9H9-7A3 LH and 9H9-7B1 LH, respectively.

The same 9H9 light chain was used in all four cases. Full sequences of the tetravalent bispecific antibodies include the following:

Key:
Bold double underlined: 9H9 Variable region
Bold single underlined: Constant domains (including embedded dotted underline)
Italic: anti-ILT4 scFv
Italic underlined: linkers
Dotted underlined: modified amino acid residues
9H9-7A3 HL
(SEQ ID NO: 141)
EVQLVESGGGLVOPGGSLRLSCAASGGIISTYWMSWVRQAPGKGLEWVANIKQ
DGSEKYYVDSVKGRFTISRDNAKNSLYLQMNSLRVEDTAMYYCARDRPVAGAS
ALWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDK

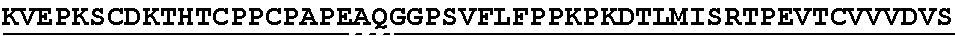

HEDPEVKFNWYVDGVEVHNAKTKPREEQYNSTYRVVSVLTVLHQDWLNGKEY

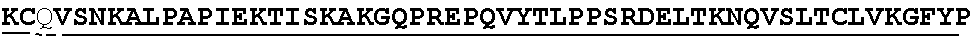

SDIAVEWESNGQPENNYKTTPPVLDSDGSFFLYSKLTVDKSRWQQGNVFSCSVM
HEALHNHYTQKSLSLSPGKGSSGGGGSQVQLVQSGAEVKKPGASVKVSCKASGYSFT

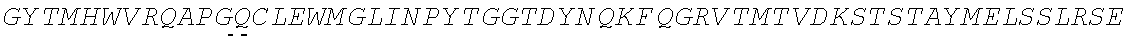

DTAVYYCARERPGGSQFIYYYPMDYWGQGTTVTVSSGGGGSGGGGSGGGGSGGGGSDIQ
MTQSPSSLSASVGDRVTITCRASANIYSYLAWYQQKPGKAPKFLVYNAITLAEGVPSRFSGS

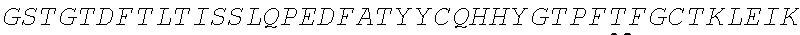

9H9-7A3 LH
(SEQ ID NO: 142)
EVQLVESGGGLVOPGGSLRLSCAASGGIISTYWMSWVRQAPGKGLEWVANIKQ
DGSEKYYVDSVKGRFTISRDNAKNSLYLQMNSLRVEDTAMYYCARDRPVAGAS
ALWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDK

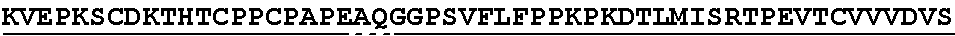

HEDPEVKFNWYVDGVEVHNAKTKPREEQYNSTYRVVSVLTVLHQDWLNGKEY

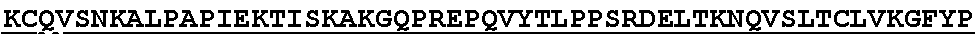

SDIAVEWESNGQPENNYKTTPPVLDSDGSFFLYSKLTVDKSRWQQGNVFSCSVM
HEALHNHYTQKSLSLSPGKGSSGGGGSDIQMTQSPSSLSASVGDRVTITCRASANIYSYL
AWYQQKPGKAPKFLVYNAITLAEGVPSRFSGSGSGTDFTLTISSLQPEDFATYYCQHHYGT

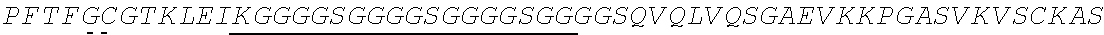

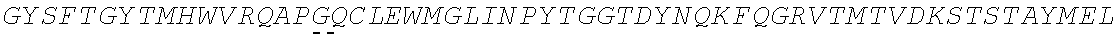

SSLRSEDTAVYYCARERPGGSQFIYYYPMDYWGQGTTVTVSS
9H9-7B1 HL
(SEQ ID NO: 143)
EVQLVESGGGLVQPGGSLRLSCAASGGIISTYWMSWVRQAPGKGLEWVANIKQ
DGSEKYYVDSVKGRFTISRDNAKNSLYLQMNSLRVEDTAMYYCARDRPVAGAS
ALWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDK

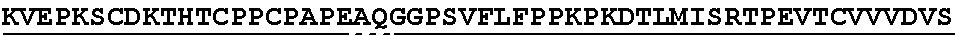

HEDPEVKFNWYVDGVEVHNAKTKPREEQYNSTYRVVSVLTVLHQDWLNGKEY

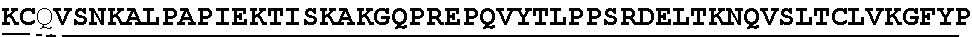

SDIAVEWESNGQPENNYKTTPPVLDSDGSFFLYSKLTVDKSRWQQGNVFSCSVM
HEALHNHYTQKSLSLSPGKGSSGGGGSQVQLVQSGAEVKKPGASVKVSCKASGYSFT

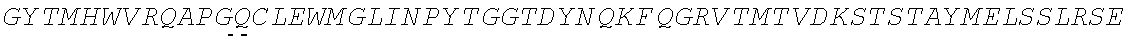

DTAVYYCARERPGGSQFIYYYALDYWGQGTTVTVSSGGGGSGGGGSGGGGSGGGGSDIQ
MTQSPSSLSASVGDRVTITCRASENIYSYLAWYQQKPGKAPKFLVYNADTLAEGVPSRFSGS

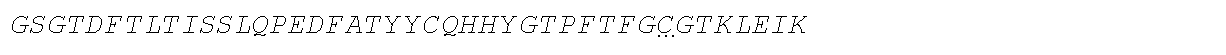

9H9-7B1 LH
(SEQ ID NO: 144)
EVQLVESGGGLVOPGGSLRLSCAASGGIISTYWMSWVRQAPGKGLEWVANIKQ
DGSEKYYVDSVKGRFTISRDNAKNSLYLQMNSLRVEDTAMYYCARDRPVAGAS
ALWGQGTLVTVSSASTKGPSVFPLAPSSKSTSGGTAALGCLVKDYFPEPVTVSW
NSGALTSGVHTFPAVLQSSGLYSLSSVVTVPSSSLGTQTYICNVNHKPSNTKVDK

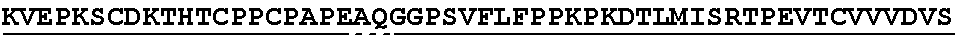

HEDPEVKFNWYVDGVEVHNAKTKPREEQYNSTYRVVSVLTVLHQDWLNGKEY

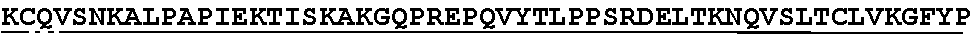

SDIAVEWESNGQPENNYKTTPPVLDSDGSFFLYSKLTVDKSRWQQGNVFSCSVM
HEALHNHYTQKSLSLSPGKGSSGGGGSDIQMTQSPSSLSASVGDRVTITCRASENIYSYL
AWYQQKPGKAPKFLVYNADTLAEGVPSRFSGSGSGTDFTLTISSLQPEDFATYYCQHHYG

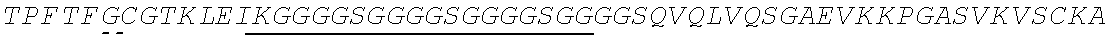

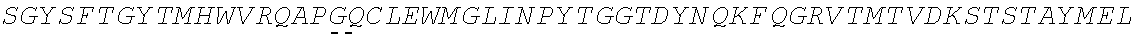

SSLRSEDTAVYYCARERPGGSQFIYYYALDYWGQGTTVTVSS
9H9 (light chain)
(SEQ ID NO: 145)
DIQMTQSPSTLSASVGDRVTITCRASQSISGWLAWYQQKPGKAPKLLIYKASSLE
SGVPSRFSGSGSGTEFTLTISSLQPDDFATYYCQQYYGSSRTFGQGTNVEIKRTV
AAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC

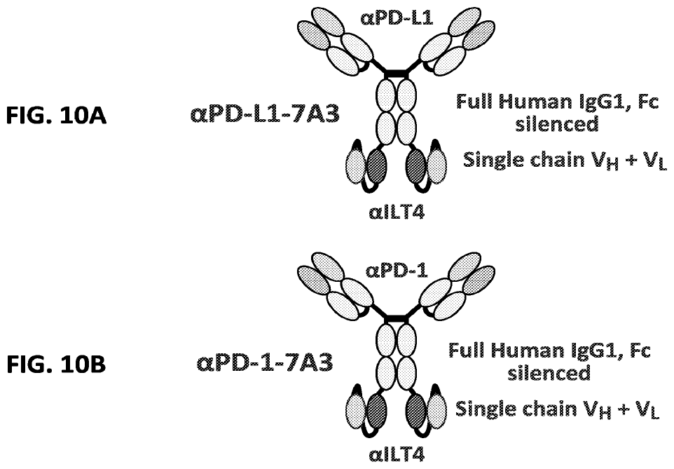

Bispecific constructs were expressed in CHO cell lines. FIG. 10A shows a schematic depiction of the bispecific constructs. FIG. 10B shows an alternative bispecific construct with an anti-PD-1 antibody in place of the anti-PD-L1 antibody.

### Example 12: Determination of Affinity and Rate Constants of Humanized Bispecific Antibodies by Bio-Layer Interferometry (BLI)

Binding affinity and binding kinetics of various human ILT4 bispecific antibodies were examined by bio-layer interferometry (BLI) using an Octet™ QK^{e} instrument (ForteBio Sartorius, Fremont, CA) according to the manufacturer's guidelines. Purified bispecific antibodies (9H9-7A3 HL, 9H9-7A3 LH, 9H9-7B1 HL, 9H9-7B1 LH, as described in Example 11) were captured on Anti-Human Fc Capture (AHC) biosensors (Fortebio Product No. 18-5060). Each antibody was prepared in dilution buffer (10mMPO4+150 mM NaCl+1 mg/mL BSA+0.05% Tween 20, pH 7.2) to 0.5 μg/mL and loaded on freshly hydrated and pre-conditioned AHC biosensors for 300 seconds at 30° C. and 1000 rpm plate shake speed. For one assay, eight biosensors were loaded with the same antibody.

Binding was determined by exposing six of the antibody loaded biosensors to analyte: soluble human ILT4-HIS (Celldex in-house reagent). Affinity measurements were determined using 2-fold serial dilutions of analyte ranging from 25 to 0.4 nM in dilution buffer at 30° C. and 1000 rpm plate shake speed. Association of the antibody loaded biosensors in analyte wells was carried out for 300 seconds, the biosensors were then moved to dilution buffer wells for 1500 sec for dissociation measurements.

Corresponding controls were conducted in each case by keeping the remaining biosensor with captured antibody in dilution buffer wells for association and dissociation steps. The data for the control biosensor was used to subtract background and account for biosensor drift and antibody dissociation from the biosensors.

Fortebio's Data Analysis Software version 8.2.0.7 (ForteBio Sartorius, Fremont, CA) was used in each case to derive kinetic parameters from the concentration series of analyte in dilution buffer binding to captured antibody. The association and dissociation curves were fitted to a 1:1 binding model using the data analysis software according to the manufacturer's guidelines.

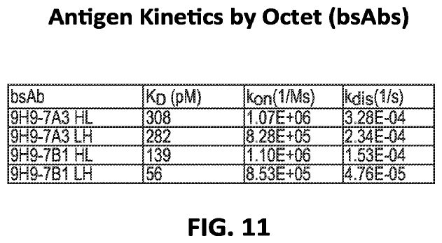

The affinity and kinetic parameters (with background subtracted) as determined are shown in FIG. 11, where kₒₙ=rate of association, k_{dis}=rate of dissociation, and K_{D}=affinity constant, determined by the ratio k_{dis}/kₒₙ.

### Example 13: Binding of Humanized Bispecific Antibodies to Human PD-L1 Using ELISA

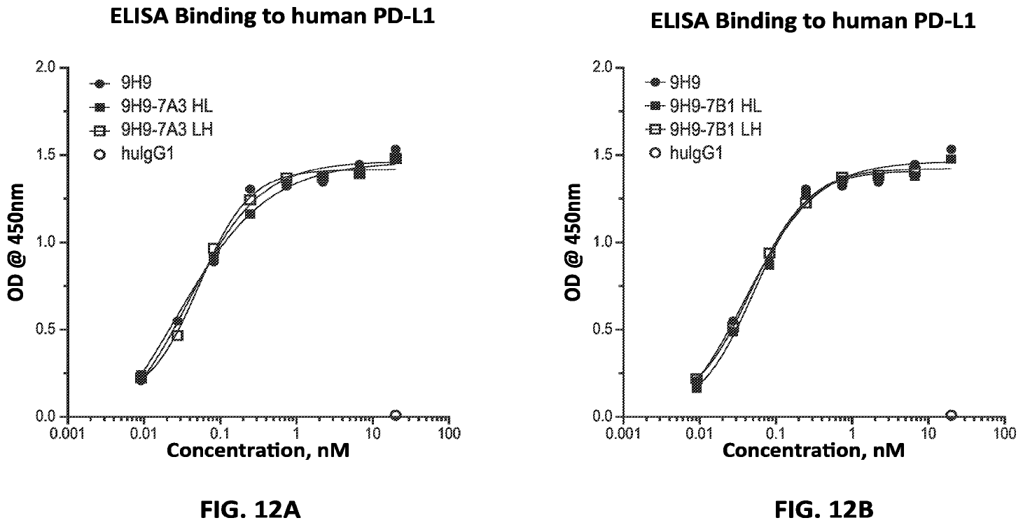

Microtiter plates were coated with recombinant human PD-L1-msFc in PBS, and then blocked with 5% bovine serum albumin in PBS. Protein A purified anti-PD-L1 monoclonal antibody 9H9, bispecific antibodies 9H9-7A3 HL, 9H9-7A3 LH, 9H9-7B1 HL, 9H9-7B1 LH (as described in Example 11) and isotype controls were added at various concentrations and incubated at 37° C. The plates were washed with PBS/Tween and then incubated with a goat-anti-human IgG Fc-specific polyclonal reagent conjugated to horseradish peroxidase at 37° C. After washing, the plates were developed with HRP substrate, and analyzed at OD 450 using a microtiter plate reader. Representative binding curves are shown in FIGS. 12A and 12B.

### Example 14: Binding of Humanized Bispecific Antibodies to Cells Expressing Human PD-L1

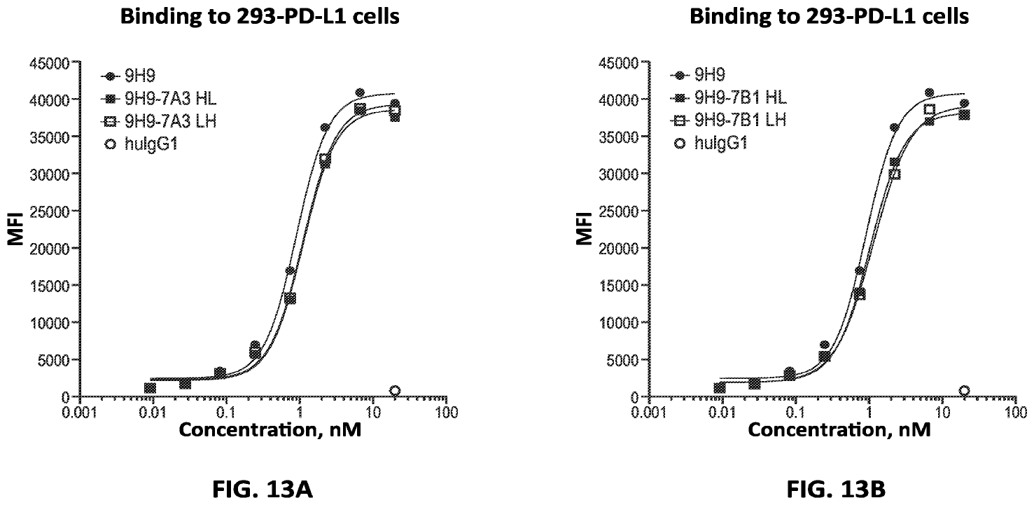

Protein A purified monoclonal antibodies, bispecific antibodies (9H9-7A3 HL, 9H9-7A3 LH, 9H9-7B1 HL, 9H9-7B1 LH, as described in Example 11) and isotype controls were incubated with HEK293 cells expressing human PD-L1 at room temperature on a plate shaker. After 20 minutes, the cells were washed with PBS containing 0.1% BSA and 0.05% NaN₃ (PBA) and the bound antibodies were detected by incubating the cells with a PE labeled goat anti-human IgG Fc-specific probe. The excess probe was washed from the cells with PBA and the cell associated fluorescence was determined by analysis using a FACSCanto II™ instrument (BD Biosciences, NJ, USA) according to the manufacturer's directions. Representative binding curves are shown in FIGS. 13A and 13B.

### Example 15: Binding of Humanized Bispecific Antibodies to Cells Expressing Human ILT4

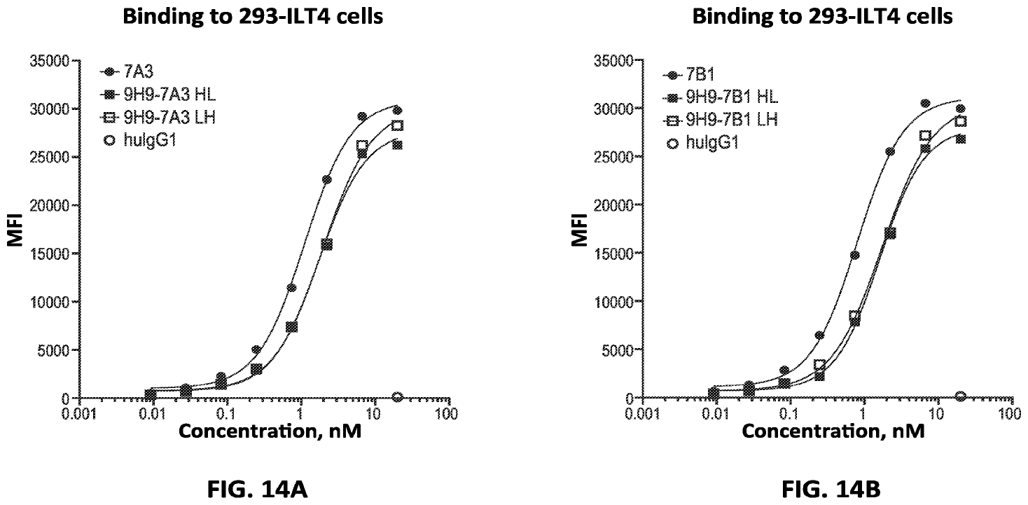

Protein A purified monoclonal antibodies, bispecific monoclonal antibodies (9H9-7A3 HL, 9H9-7A3 LH, 9H9-7B1 HL, 9H9-7B1 LH, as described in Example 11) and isotype controls were incubated with HEK293 cells expressing human ILT4 at room temperature on a plate shaker. After 20 minutes, the cells were washed with PBS containing 0.1% BSA and 0.05% NaN₃ (PBA) and the bound antibodies were detected by incubating the cells with a PE labeled goat anti-human IgG Fc-specific probe. The excess probe was washed from the cells with PBA and the cell associated fluorescence was determined by analysis using a FACSCanto II™ instrument (BD Biosciences, NJ, USA) according to the manufacturer's directions. Representative binding curves are shown in FIGS. 14A and 14B.

### Example 16: Bifunctional Binding of Humanized Bispecific Antibodies to Cell-Expressed Human ILT4 and to Human PD-L1

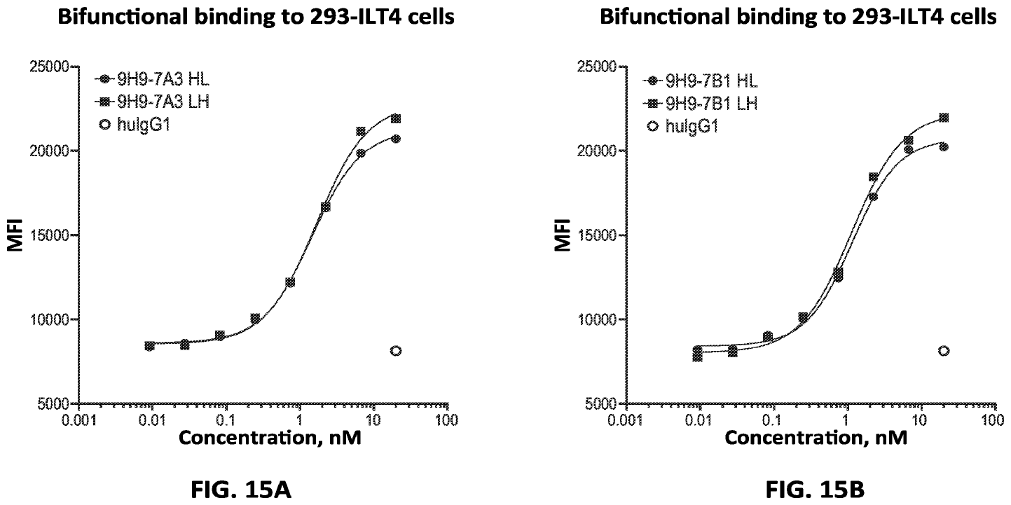

Binding of bispecific constructs to ILT4 and PD-L1 was assessed using HEK293 cells expressing human ILT4. In brief, dilutions of the bispecific constructs were allowed to bind to the ILT4 expressing cell before adding human PD-L1-msFc that was detected with PE labeled goat anti-mouse IgG Fc-specific probe. Representative binding curves for four bispecific constructs (9H9-7A3 HL, 9H9-7A3 LH, 9H9-7B1 HL, and 9H9-7B1 LH, as described in Example 11) are shown in FIGS. 15A and 15B. All four bispecific antibodies demonstrated significant binding to both ILT4 and PD-L1.

### Example 17: T Cell PD1/PD-L1 Blockade of Humanized Bispecific Antibodies

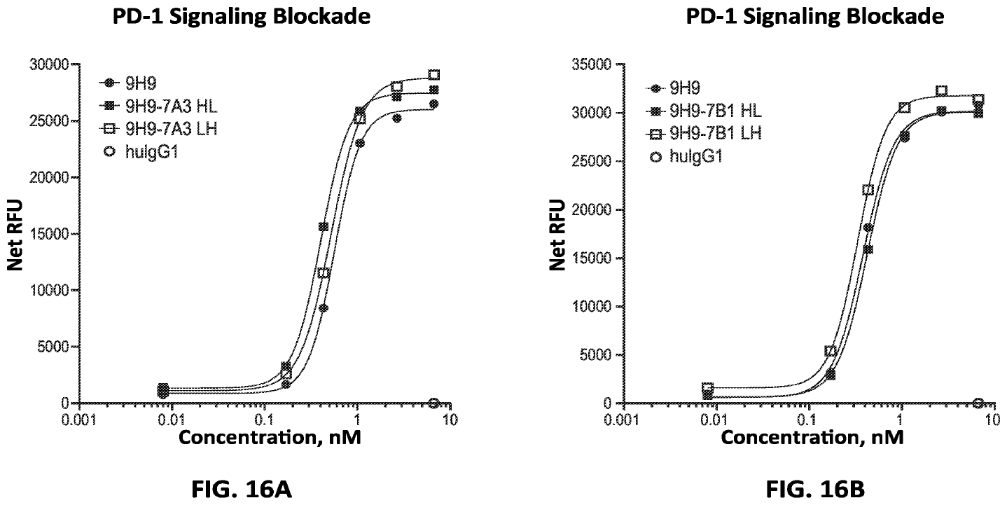

The effect of the bispecific antibodies (9H9-7A3 HL, 9H9-7A3 LH, 9H9-7B1 HL, 9H9-7B1 LH, as described in Example 11) on blockade of the PD1/PD-L1 interaction was determined using the commercially available PD-1/PD-L1 Blockade Assay from Promega®. Two engineered cell lines, PD1 Effector cells and PD-L1 aAPC/CHO-K1 cells were co-cultured in the presence of the antibodies for 6 hours. Blocking of the PD1/PD-L1 interaction results in TCR activation and induces luminescence via the NFAT pathway. Luminescence was detected by the addition of Bio-Glo reagent and quantitated on a Perkin Elmer® Victor X4 luminometer. As shown in FIGS. 16A and 16B, the anti-PD-L1 antibodies and bispecific antibodies effectively block the PD1/PD-L1 interaction between cells leading to activation of the NFAT pathway.

### Example 18: Induction of TNF-α Production by Humanized Bispecific Antibodies in Macrophages

Macrophages were derived from human monocytes as follows: PBMC's were added to a T175 cm² flasks and monocytes allowed to adhere for ˜2 hours at 37° C., 6% CO₂. The non-adherent cells were removed and the monocytes cultured for 7 days in RPMI containing 10% FBS and 50 ng/mL M-CSF (R&D Systems).

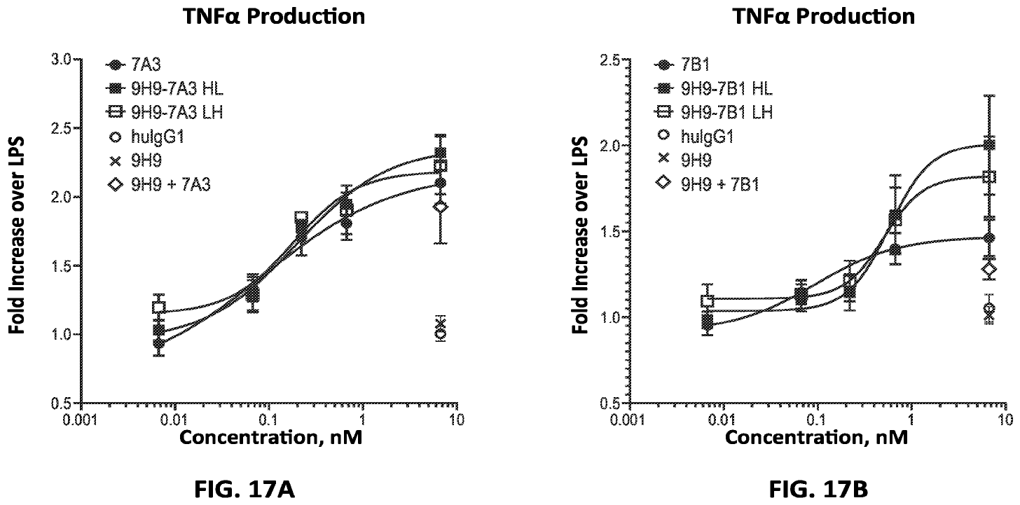

The cells were then incubated in the presence of the bispecific antibodies (9H9-7A3 HL, 9H9-7A3 LH, 9H9-7B1 HL, 9H9-7B1 LH, as described in Example 11) and the appropriate antibody controls with 50 ng/mL LPS (Invivogen) at 37° C., 6% CO₂. After 24 hours, the cells were harvested and the supernatant was collected and stored for cytokine analysis. Induction of TNF-α was evaluated in the supernatants collected by ELISA (R&D Systems). FIGS. 17A and 17B show the increase in TNF-α production with the bispecific antibodies.

### Example 19: Inhibition of HLA-G Binding to ILT4 by Humanized Bispecific Antibodies

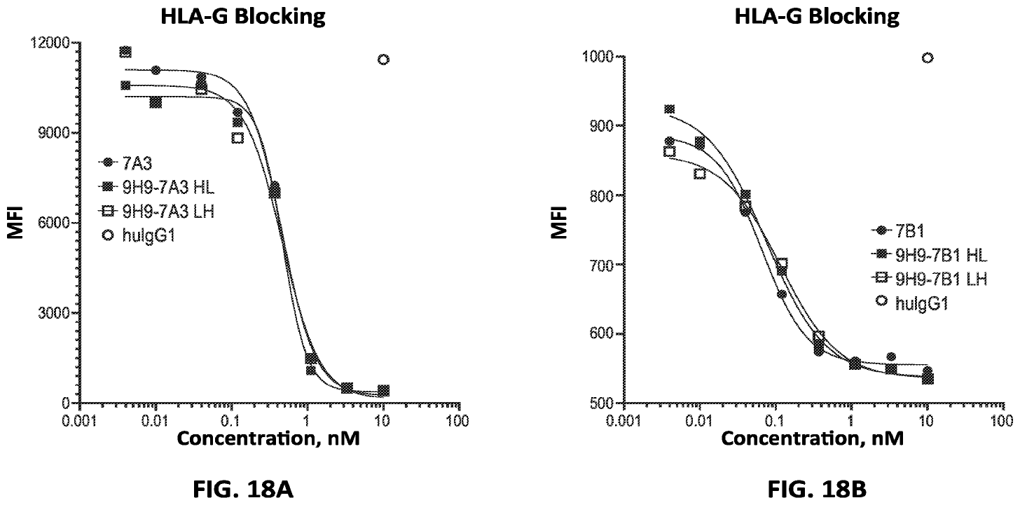

Dilutions of the bispecific antibodies (9H9-7A3 HL, 9H9-7A3 LH, 9H9-7B1 HL, 9H9-7B1 LH, as described in Example 11) and antibody controls were incubated on HEK293 cells expressing human ILT4 at room temperature on a plate shaker. After 30 minutes, the cells were washed and PE-labeled HLA-G tetramer (FredHutch) was added. After an additional 30 minutes, the cells were washed with PBA and the cell associated fluorescence was determined by analysis using a FACSCanto II™ instrument (BD Biosciences, NJ, USA) according to the manufacturer's directions. Representative blocking curves are shown in FIGS. 18A and 18B.

TABLE 5
SUMMARY OF SEQUENCE LISTING
7A3 and 7B1 Humanized Sequences

| | |
|---|---|
| SEQ ID NO: 1 | GYTIH |
| 7A3 V_{H} CDR1 | |
| (Kabat) | |
| SEQ ID NO: 2 | GYSFTGY |
| 7A3 V_{H} CDR1 | |
| (Chothia) | |
| SEQ ID NO: 3 | LINPYTGGTDYNQKFKG |
| 7A3 V_{H} CDR2 | |
| (Kabat) | |
| SEQ ID NO: 4 | NPYTGG |
| 7A3 V_{H} CDR2 | |
| (Chothia) | |
| SEQ ID NO: 5 | ERPGGSQFIYYYPMDY |
| 7A3 V_{H} CDR3 | |
| SEQ ID NO: 6 | RASANIYSYLA |
| 7A3 V_{L} CDR1 | |
| SEQ ID NO: 7 | NAITLAE |
| 7A3 V_{L} CDR2 | |
| SEQ ID NO: 8 | QHHYGTPFT |
| 7A3 V_{L} CDR3 | |
| SEQ ID NO: 9 | QVQLVQSGAEVKKPGASVKVSCKASGYSFTGYTMHWVRQAPGQGLE |
| 7A3 VH | WMGLINPYTGGTDYNQKFQGRVTMTVDKSTSTAYMELSSLRSEDTAV |
| Hu-VH2 | YYCARERPGGSQFIYYYPMDYWGQGTTVTVSS |
| SEQ ID NO: 10 | DIQMTQSPSSLSASVGDRVTITCRASANIYSYLAWYQQKPGKAPKFLVY |
| 7A3 VL | NAITLAEGVPSRESGSGSGTDFTLTISSLQPEDFATYYCQHHYGTPFTFG |
| Hu-VL1 | GGTKLEIK |
| SEQ ID NO: 11 | GYTMH |
| 7B1 V_{H} CDR1 | |
| (Kabat) | |
| SEQ ID NO: 12 | GYSFTGY |
| 7B1 V_{H} CDR1 | |
| (Chothia) | |
| SEQ ID NO: 13 | LINPYTGGTDYNQKFKG |
| 7B1 V_{H} CDR2 | |
| (Kabat) | |
| SEQ ID NO: 14 | NPYTGG |
| 7B1 V_{H} CDR2 | |
| (Chothia) | |
| SEQ ID NO: 15 | ERPGGSQFIYYYALDY |
| 7B1 V_{H} CDR3 | |
| SEQ ID NO: 16 | RASENIYSYLA |
| 7B1 V_{L} CDR1 | |
| SEQ ID NO: 17 | NADTLAE |
| 7B1 V_{L} CDR2 | |
| SEQ ID NO: 18 | QHHYGTPFT |
| 7B1 V_{L} CDR3 | |
| SEQ ID NO: 19 | QVQLVQSGAEVKKPGASVKVSCKASGYSFTGYTMHWVRQAPGQGLE |
| 7B1 VH | WMGLINPYTGGTDYNQKFQGRVTMTVDRSTSTAYMELSSLRSEDTAV |
| Hu-VH2 | YYCARERPGGSQFIYYYALDYWGQGTTVTVSS |
| SEQ ID NO: 20 | DIQMTQSPSSLSASVGDRVTITCRASENIYSYLAWYQQKPGKAPKFLVY |
| 7B1 VL | NADTLAEGVPSRFSGSGSGTDFTLTISSLQPEDFATYYCQHHYGTPFTFG |
| Hu-VL1 | GGTKLEIK |
| SEQ ID NO: 21 | G Y T (I, M) H |
| V_{H} CDR1 | |
| Consensus | |
| SEQ ID NO: 22 | E R P G G S Q F I Y Y Y (P, A) (M, L) DY |
| V_{H} CDR3 | |
| Consensus | |
| SEQ ID NO: 23 | R A S (A,E) N I Y S Y L A |
| V_{L} CDR1 | |
| Consensus | |
| SEQ ID NO: 24 | N A (I, D) T L A E |
| V_{L} CDR2 | |
| Consensus | |
| SEQ ID NO: 25 | QVQLVQSGAEVKKPGASVKVSCKASGYSFTGYTMHWVRQAPGQGLEWMG |
| 7A3 AQQ HC | LINPYTGGTDYNQKFQGRVTMTVDKSTSTAYMELSSLRSEDTAVYYCARER |
| amino acids | PGGSQFIYYYPMDYWGQGTTVTVSSASTKGPSVFPLAPSSKSTSGGTAALGCLV |
| | KDYFPEPVTVSWNSGALTSGVHTFPAVLQSSGLYSLSSVVTVPSSSLGTQTYICNVN |
| | HKPSNTKVDKKVEPKSCDKTHTCPPCPAPE [symbol] GGPSVFLFPPKPKDTLMISRTPE |
| | VTCVVVDVSHEDPEVKFNWYVDGVEVHNAKTKPREEQYNSTYRVVSVLTVLHQD |
| | WLNGKEYKC [symbol] VSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQQGNVF |
| | SCSVMHEALHNHYTQKSLSLSPG |
| SEQ ID NO: 26 | DIQMTQSPSSLSASVGDRVTITCRASANIYSYLAWYQQKPGKAPKFLVYNAI |
| 7A3 LC amino | TLAEGVPSRFSGSGSGTDFTLTISSLQPEDFATYYCQHHYGTPFTFGGGTKL |
| acids | EIKRTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGEC |
| SEQ ID NO: 27 | QVQLVQSGAEVKKPGASVKVSCKASGYSFTGYTMHWVRQAPGQGLEWMG |
| 7B1 AQQ HC | LINPYTGGTDYNQKFQGRVTMTVDRSTSTAYMELSSLRSEDTAVYYCARER |
| amino acids | PGGSQFIYYYALDYWGQGTTVTVSSASTKGPSVFPLAPSSKSTSGGTAALGCLV |
| | KDYFPEPVTVSWNSGALTSGVHTFPAVLQSSGLYSLSSVVTVPSSSLGTQTYICNVN |
| | HKPSNTKVDKKVEPKSCDKTHTCPPCPAPE [symbol] GGPSVFLFPPKPKDTLMISRTPE |
| | VTCVVVDVSHEDPEVKFNWYVDGVEVHNAKTKPREEQYNSTYRVVSVLTVLHQD |
| | WLNGKEYKC [symbol] VSNKALPAPIEKTISKAKGQPREPQVYTLPPSRDELTKNQVSLTCL |
| | VKGFYPSDIAVEWESNGQPENNYKTTPPVLDSDGSFFLYSKLTVDKSRWQQGNVF |
| | SCSVMHEALHNHYTQKSLSLSPG |
| SEQ ID NO: 28 | DIQMTQSPSSLSASVGDRVTITCRASENIYSYLAWYQQKPGKAPKFLVYNA |
| 7B1 LC amino | DTLAEGVPSRFSGSGSGTDFTLTISSLQPEDFATYYCQHHYGTPFTFGGGTK |
| acids | LEIKRTVAAPSVFIFPPSDEQLKSGTASVVCLLNNFYPREAKVQWKVDNALQSGNS |
| | QESVTEQDSKDSTYSLSSTLTLSKADYEKHKVYACEVTHQGLSSPVTKSFNRGEC |

PD-L1 Antibody Sequences

| | |
|---|---|
| SEQ ID NO: 29 | TSWMS |
| 7H7 V_{H} CDR1 | |
| SEQ ID NO: 30 | NIKQDGSEKYYVDSVKG |
| 7H7 V_{H} CDR2 | |
| SEQ ID NO: 31 | DRPVAGASAL |
| 7H7 V_{H} CDR3 | |
| SEQ ID NO: 32 | RASQSISGWLA |
| 7H7 V_{L} CDR1 | |
| SEQ ID NO: 33 | KASSLES |
| 7H7 V_{L} CDR2 | |
| SEQ ID NO: 34 | QQYYGSSRT |
| 7H7 V_{L} CDR3 | |
| SEQ ID NO: 35 | TSWMS |
| 1B3 V_{H} CDR1 | |
| SEQ ID NO: 36 | NIKQDGSEKYYVDSVKG |
| 1B3 V_{H} CDR2 | |
| SEQ ID NO: 37 | DRPVAGASAL |
| 1B3 V_{H} CDR3 | |
| SEQ ID NO: 38 | RASQSISGWLA |
| 1B3 V_{L} CDR1 | |
| SEQ ID NO: 39 | KASSLES |
| 1B3 V_{L} CDR2 | |
| SEQ ID NO: 40 | QQYYGSSRT |
| 1B3 V_{L} CDR3 | |
| SEQ ID NO: 41 | TYWMS |
| 3B6 V_{H} CDR1 | |
| SEQ ID NO: 42 | NIKQDGSEKYYVDSVKG |
| 3B6 V_{H} CDR2 | |
| SEQ ID NO: 43 | DRPVAGASAL |
| 3B6 V_{H} CDR3 | |
| SEQ ID NO: 44 | RASQSISGWLA |
| 3B6 V_{L} CDR1 | |
| SEQ ID NO: 45 | KASSLES |
| 3B6 V_{L} CDR2 | |
| SEQ ID NO: 46 | QQYYGSSRT |
| 3B6 V_{L} CDR3 | |
| SEQ ID NO: 47 | THWMS |
| 8B1 V_{H} CDR1 | |
| SEQ ID NO: 48 | NIKQDGSEKYYVDSVKG |
| 8B1 V_{H} CDR2 | |
| SEQ ID NO: 49 | DRPVAGASAL |
| 8B1 V_{H} CDR3 | |
| SEQ ID NO: 50 | RASQSISGWLA |
| 8B1 V_{L} CDR1 | |
| SEQ ID NO: 51 | KASSLES |
| 8B1 V_{L} CDR2 | |
| SEQ ID NO: 52 | QQYYGSSRT |
| 8B1 V_{L} CDR3 | |
| SEQ ID NO: 53 | SSWMS |
| 4A3 V_{H} CDR1 | |
| SEQ ID NO: 54 | NIKQDGSEKYYVDSVKG |
| 4A3 V_{H} CDR2 | |
| SEQ ID NO: 55 | DRPVAGASAL |
| 4A3 V_{H} CDR3 | |
| SEQ ID NO: 56 | RASQSISGWLA |
| 4A3 V_{L} CDR1 | |
| SEQ ID NO: 57 | KASSLES |
| 4A3 V_{L} CDR2 | |
| SEQ ID NO: 58 | QQYYGSSRT |
| 4A3 V_{L} CDR3 | |
| SEQ ID NO: 59 | TYWMS |
| 9H9 V_{H} CDR1 | |
| SEQ ID NO: 60 | NIKQDGSEKYYVDSVKG |
| 9H9 V_{H} CDR2 | |
| SEQ ID NO: 61 | DRPVAGASAL |
| 9H9 V_{H} CDR3 | |
| SEQ ID NO: 62 | RASQSISGWLA |
| 9H9 V_{L} CDR1 | |
| SEQ ID NO: 63 | KASSLES |
| 9H9 V_{L} CDR2 | |
| SEQ ID NO: 64 | QQYYGSSRT |
| 9H9 V_{L} CDR3 | |
| SEQ ID NO: 77 | EVQLVESGGGLVQPGGSLRLSCAASGGTISTSWMSWVRQAPGKGLEW |
| 7H7 V_{H}- | VANIKQDGSEKYYVDSVKGRFTISRDNAKNSLYLQMNSLRVEDTAIYY |
| without signal | CARDRPVAGASALWGQGTLVTVSS |
| sequence | |
| SEQ ID NO: 78 | DIQMTQSPSTLSASVGDRVTITCRASQSISGWLAWYQQKQGKAPKLLIY |
| 7H7 V_{L}- | KASSLESGVPSRFSGSGSGTEFTLTISSLQPDDFATYYCQQYYGSSRTFG |
| without signal | QGTNVEIK |
| sequence | |
| SEQ ID NO: 79 | EVQLVESGGGLVQPGGSLRLSCAASGGTISTSWMSWVRQAPGKGLEW |
| 1B3 V_{H-} | VANIKQDGSEKYYVDSVKGRFTISRDNAKNSLYLQMNSLRVEDTAMY |
| without signal | YCARDRPVAGASALWGQGTLVTVSS |
| sequence | |
| SEQ ID NO: 80 | DIQMTQSPSTLSASVGDRVTITCRASQSISGWLAWYQQKPGKAPKLLIY |
| 1B3 V_{L-} | KASSLESGVPSRFSGSGSGTEFTLTISSLQPDDFATYYCQQYYGSSRTFG |
| without signal | QGTNVEIK |
| sequence | |
| SEQ ID NO: 81 | EVQLVESGGGLVQPGGSLRLSCAASGGTTSTYWMSWVRQAPGKGLE |
| 3B6 V_{H-} | WVANIKQDGSEKYYVDSVKGRFTISRDNAKNSLNLQMNSLRVEDTAIY |
| without signal | YCARDRPVAGASALWGQGTLVTVSS |
| sequence | |
| SEQ ID NO: 82 | DIQMTQSPSTLSASVGDRVTITCRASQSISGWLAWYQQKPGKAPKLLIY |
| 3B6 V_{L-} | KASSLESGVPSRFSGSGSGTEFTLTISSLQPDDFATYYCQQYYGSSRTFG |
| without signal | QGTNVEIK |
| sequence | |
| SEQ ID NO: 83 | EVRLVESGGGLVQPGGSLRLSCAASGDIISTHWMSWVRQAPGKGLEW |
| 8B1 V_{H-} | VANIKQDGSEKYYVDSVKGRFTISRDNAKNSLYLQMNTLRVEDTAIYY |
| without signal | CTRDRPVAGASALWGQGTLVTVSS |
| sequence | |
| SEQ ID NO: 84 | DIQMTQSPSTLSASVGDRVTITCRASQSISGWLAWYQQKPGKAPKLLIY |
| 8B1 V_{L-} | KASSLESGVPLRFSGSGSGTEFTLTISSLQPDDFATYYCQQYYGSSRTFG |
| without signal | QGTNVEIK |
| sequence | |
| SEQ ID NO: 85 | EVQLVESGGGLVQPGGSLRLSCAASGGIISSSWMSWVRQAPGKGLEWV |
| 4A3 V_{H-} | ANIKQDGSEKYYVDSVKGRFTISRDNAKDLLYLQMNSLRVEDTALYYC |
| without signal | ARDRPVAGASALWGQGTLVTVSS |
| sequence | |
| SEQ ID NO: 86 | DIQMTQSPSTLSASVGDRVTITCRASQSISGWLAWYQQKPGKAPKLLIY |
| 4A3 V_{L-} | KASSLESGVPSRFSGSGSGTEFTLTISSLQPDDFATYYCQQYYGSSRTFG |
| without signal | QGTNVEIK |
| sequence | |
| SEQ ID NO: 87 | EVQLVESGGGLVQPGGSLRLSCAASGGIISTYWMSWVRQAPGKGLEW |
| 9H9 VH- | VANIKQDGSEKYYVDSVKGRFTISRDNAKNSLYLQMNSLRVEDTAMY |
| without signal | YCARDRPVAGASALWGQGTLVTVSS |
| sequence | |
| SEQ ID NO: 88 | DIQMTQSPSTLSASVGDRVTITCRASQSISGWLAWYQQKPGKAPKLLIY |
| 9H9 V_{L-} | KASSLESGVPSRFSGSGSGTEFTLTISSLQPDDFATYYCQQYYGSSRTFG |
| without signal | QGTNVEIK |
| sequence | |
| SEQ ID NO: 89 | MTPIVTVLIC LGLSLGPRTH VQTGTIPKPT LWAEPDSVIT |
| ILT4 | QGSPVTLSCQ GSLEAQEYRL YREKKSASWI TRIRPELVKN |
| Isoform 1 | GQFHIPSITW EHTGRYGCQY YSRARWSELS DPLVLVMTGA |
| (Q8N423-1) | YPKPTLSAQP SPVVTSGGRV TLQCESQVAF GGFILCKEGE |
| | EEHPQCLNSQ PHARGSSRAI FSVGPVSPNR RWSHRCYGYD |
| | LNSPYVWSSP SDLLELLVPG VSKKPSLSVQ PGPVVAPGES |
| | LTLQCVSDVG |
| | YDRFVLYKEG ERDLRQLPGR QPQAGLSQAN FTLGPVSRSY |
| | GGQYRCYGAH NLSSECSAPS DPLDILITGQ IRGTPFISVQ |
| | PGPTVASGEN VTLLCQSWRQ FHTFLLTKAG AADAPLRLRS |
| | IHEYPKYQAE FPMSPVTSAH AGTYRCYGSL NSDPYLLSHP |
| | SEPLELVVSG PSMGSSPPPT GPISTPAGPE DQPLTPTGSD |
| | PQSGLGRHLG VVIGILVAVV LLLLLLLLLF LILRHRRQGK |
| | HWTSTQRKAD FQHPAGAVGP EPTDRGLQWR SSPAADAQEE |
| | NLYAAVKDTQ PEDGVEMDTR AAASEAPQDV TYAQLHSLTL |
| | RRKATEPPPS QEREPPAEPS IYATLAIH |

ILT4 Nucleotide Sequences
Key for SEQ ID Nos: 90-93
Bold: Variable region
Italic: Constant domains
Dotted Underline: modified amino acids/bases

| | |
|---|---|
| SEQ ID NO: 90 | CAAGTGCAGCTGGTGCAGAGCGGCGCTGAGGTGAAGAAGCCCGGCGCCAGCGTC |
| 7A3 AQQ HC | AAGGTCAGCTGTAAGGCCAGCGGCTACAGCTTCACTGGCTACACTATGCACTGGGT |
| DNA | GAGACAAGCCCCCGGCCAAGGACTGGAGTGGATGGGACTGATCAACCCTTACACT |
| | GGCGGCACTGACTACAACCAGAAATTCCAAGGAAGGGTCACAATGACTGTGGACA |
| | AGAGCACATCCACTGCCTACATGGAGCTGAGCTCTCTGAGGAGCGAGGACACAGC |
| | CGTGTACTACTGTGCCAGAGAGAGACCCGGCGGCAGCCAGTTCATCTACTACTACC |
| | CTATGGACTACTGGGGCCAAGGCACAACAGTGACTGTGAGCAGCGCTAGCACAAA |
| | GGGCCCTTCCGTGTTTCCACTGGCTCCCAGCTCTAAGTCCACCAGCGGAGGAACAGC |
| | CGCTCTGGGCTGTCTGGTGAAGGACTATTTCCCAGAGCCCGTGACCGTGAGCTGGAA |
| | CTCTGGCGCCCTGACCAGCGGAGTGCATACATTTCCTGCTGTGCTGCAGTCCAGCGG |
| | CCTGTACTCTCTGTCTTCCGTGGTGACCGTGCCAAGCTCTTCCCTGGGCACCCAGACA |
| | TATATCTGCAACGTGAATCACAAGCCATCCAATACAAAGGTGGACAAGAAGGTGGA |
| | GCCCAAGAGCTGTGATAAGACCCATACATGCCCCCCTTGTCCTGCTCCAGAG [symbol] |
| | [symbol] GAGGACCATCCGTGTTCCTGTTTCCACCCAAGCCTAAGGACACCCTGATGATCTCT |
| | AGGACCCCAGAGGTGACCTGCGTGGTGGTGGACGTGAGCCACGAGGATCCCGAGG |
| | TGAAGTTCAACTGGTACGTGGATGGCGTGGAGGTGCATAATGCCAAGACAAAGCCA |
| | AGGGAGGAGCAGTACAATAGCACCTATCGGGTGGTGTCTGTGCTGACAGTGCTGCA |
| | CCAGGACTGGCTGAACGGCAAGGAGTACAAGTGC [symbol] GTGTCTAATAAGGCCCTGC |
| | CCGCTCCTATCGAGAAGACCATCTCCAAGGCCAAGGGCCAGCCTAGGGAGCCACAG |
| | GTGTACACACTGCCTCCAAGCCGGGACGAGCTGACCAAGAACCAGGTGTCTCTGACA |
| | TGTCTGGTGAAGGGCTTCTATCCCTCTGATATCGCTGTGGAGTGGGAGTCCAATGGC |
| | CAGCCTGAGAACAATTACAAGACCACACCCCCTGTGCTGGACTCCGATGGCAGCTTC |
| | TTTCTGTATTCCAAGCTGACCGTGGATAAGAGCAGGTGGCAGCAGGGCAACGTGTTT |
| | TCTTGTTCCGTGATGCATGAGGCTCTGCACAATCATTACACACAGAAGAGCCTGTCTC |
| | TGTCCCCTGGC |
| SEQ ID NO: 91 | GACATCCAGATGACACAGAGCCCAAGCTCTCTGAGCGCCAGCGTGGGAGAT |
| 7A3 LC DNA | AGGGTGACAATCACATGTAGGGCCAGCGCCAACATCTACAGCTATCTGGCT |
| | TGGTACCAGCAGAAGCCCGGCAAGGCCCCTAAGTTTCTGGTGTACAACGCC |
| | ATCACTCTGGCTGAGGGCGTGCCTAGCAGATTTAGCGGCAGCGGAAGCGGC |
| | ACAGACTTCACTCTGACAATCAGCTCTCTGCAGCCAGAGGATTTCGCCACA |
| | TACTACTGCCAGCACCACTACGGCACTCCTTTCACATTCGGCGGCGGCACT |
| | AAGCTGGAGATCAAGCGTACGGTGGCCGCTCCAAGCGTGTTCATCTTTCCCCCT |
| | TCTGACGAGCAGCTGAAGTCTGGCACAGCCTCCGTGGTGTGCCTGCTGAACAACTT |
| | CTACCCCAGAGAGGCCAAGGTGCAGTGGAAGGTGGATAACGCTCTGCAGTCTGGC |
| | AATTCCCAGGAGAGCGTGACCGAGCAGGACTCTAAGGATTCCACATATAGCCTGA |
| | GCTCTACCCTGACACTGTCTAAGGCCGATTACGAGAAGCACAAGGTGTATGCTTGC |
| | GAGGTGACCCATCAGGGCCTGTCCAGCCCAGTGACAAAGTCCTTCAATCGCGGCGA |
| | GTGT |
| SEQ ID NO: 92 | CAAGTGCAGCTGGTGCAGAGCGGCGCTGAGGTGAAGAAGCCCGGCGCCAGCGTG |
| 7B1 AQQ HC | AAAGTGAGCTGTAAGGCCTCCGGCTACAGCTTCACTGGCTACACTATGCACTGGGT |
| DNA | CAGACAAGCCCCCGGCCAAGGACTGGAGTGGATGGGACTGATCAACCCTTACACT |
| | GGCGGCACTGACTACAACCAGAAGTTCCAAGGAAGGGTGACTATGACTGTGGATA |
| | GGTCCACAAGCACAGCCTACATGGAGCTGTCCTCTCTGAGATCCGAGGACACTGCC |
| | GTGTACTACTGTGCTAGGGAGAGACCCGGCGGCAGCCAGTTCATCTACTACTACGC |
| | TCTGGACTACTGGGGCCAAGGCACAACAGTCACAGTGAGCAGCGCTAGCACAAAG |
| | GGCCCTTCCGTGTTTCCACTGGCTCCCAGCTCTAAGTCCACCAGCGGAGGAACAGCC |
| | GCTCTGGGCTGTCTGGTGAAGGACTATTTCCCAGAGCCCGTGACCGTGAGCTGGAAC |
| | TCTGGCGCCCTGACCAGCGGAGTGCATACATTTCCTGCTGTGCTGCAGTCCAGCGGC |
| | CTGTACTCTCTGTCTTCCGTGGTGACCGTGCCAAGCTCTTCCCTGGGCACCCAGACAT |
| | ATATCTGCAACGTGAATCACAAGCCATCCAATACAAAGGTGGACAAGAAGGTGGAG |
| | CCCAAGAGCTGTGATAAGACCCATACATGCCCCCCTTGTCCTGCTCCAGAG [symbol] |
| | GGAGGACCATCCGTGTTCCTGTTTCCACCCAAGCCTAAGGACACCCTGATGATCTCTA |
| | GGACCCCAGAGGTGACCTGCGTGGTGGTGGACGTGAGCCACGAGGATCCCGAGGT |
| | GAAGTTCAACTGGTACGTGGATGGCGTGGAGGTGCATAATGCCAAGACAAAGCCAA |
| | GGGAGGAGCAGTACAATAGCACCTATCGGGTGGTGTCTGTGCTGACAGTGCTGCAC |
| | CAGGACTGGCTGAACGGCAAGGAGTACAAGTGC [symbol] GTGTCTAATAAGGCCCTGCC |
| | CGCTCCTATCGAGAAGACCATCTCCAAGGCCAAGGGCCAGCCTAGGGAGCCACAGG |
| | TGTACACACTGCCTCCAAGCCGGGACGAGCTGACCAAGAACCAGGTGTCTCTGACAT |
| | GTCTGGTGAAGGGCTTCTATCCCTCTGATATCGCTGTGGAGTGGGAGTCCAATGGCC |
| | AGCCTGAGAACAATTACAAGACCACACCCCCTGTGCTGGACTCCGATGGCAGCTTCTT |
| | TCTGTATTCCAAGCTGACCGTGGATAAGAGCAGGTGGCAGCAGGGCAACGTGTTTTC |
| | TTGTTCCGTGATGCATGAGGCTCTGCACAATCATTACACACAGAAGAGCCTGTCTCTG |
| | TCCCCTGGC |
| SEQ ID NO: 93 | GACATCCAGATGACACAGAGCCCAAGCTCTCTGAGCGCCAGCGTGGGAGAT |
| 7B1 LC DNA | AGGGTGACAATCACTTGTAGGGCCAGCGAGAACATCTACAGCTATCTGGC |
| | TTGGTACCAGCAGAAGCCCGGCAAGGCCCCAAAGTTTCTGGTGTACAACGC |
| | CGACACTCTCGCTGAGGGCGTCCCTTCTAGGTTTTCCGGCAGCGGCTCCGG |
| | CACTGACTTCACACTGACTATCAGCTCTCTGCAGCCAGAGGATTTCGCCAC |
| | ATACTACTGCCAGCACCACTACGGCACTCCTTTCACATTCGGCGGCGGCAC |
| | TAAGCTGGAGATCAAGCGTACGGTGGCCGCTCCAAGCGTGTTCATCTTTCCCCC |
| | TTCTGACGAGCAGCTGAAGTCTGGCACAGCCTCCGTGGTGTGCCTGCTGAACAACT |
| | TCTACCCCAGAGAGGCCAAGGTGCAGTGGAAGGTGGATAACGCTCTGCAGTCTGG |
| | CAATTCCCAGGAGAGCGTGACCGAGCAGGACTCTAAGGATTCCACATATAGCCTGA |
| | GCTCTACCCTGACACTGTCTAAGGCCGATTACGAGAAGCACAAGGTGTATGCTTGC |
| | GAGGTGACCCATCAGGGCCTGTCCAGCCCAGTGACAAAGTCCTTCAATCGCGGCGA |
| | GTGT |

Additional ILT4 Humanized Sequences

| | |
|---|---|
| SEQ ID NO: 97 | EVQLVQSGAEVKKPGASVKVSCKASGYSFTGYTIHWVRQAPGQGLEW |
| 7A3 | IGLINPYTGGTDYNQKFQGRVTLTVDKSTSTAYMELSSLRSEDTAVYYC |
| hu-VH1 | ARERPGGSQFIYYYPMDYWGQGTTVTVSS |
| SEQ ID NO: 98 | EVQLVQSGSELKKPGASVKVSCKASGYSFTGYTIHWVRQAPGQGLEWI |
| 7A3 | GLINPYTGGTDYNQGFTGRFVLSVDKSVSTAYLQISSLKAEDTAVYYC |
| hu-VH3 | ARERPGGSQFIYYYPMDYWGQGTTVTVSS |
| SEQ ID NO: 99 | QVQLVQSGSELKKPGASVKVSCKASGYSFTGYTMNWVRQAPGQGLE |
| 7A3 | WMGLINPYTGGTDYNQGFTGRFVFSVDKSVSTAYLQISSLKAEDTAVY |
| hu-VH4 | YCARERPGGSQFIYYYPMDYWGQGTTVTVSS |
| SEQ ID NO: 100 | DIQMTQSPSSLSASVGDRVTITCRASANIYSYLAWYQQKPGKAPKFLVY |
| 7A3 | NAITLASGVPSRESGSGSGTDFTLTISSLQPEDFATYYCQHHYGTPFTFG |
| hu-VL2 | GGTKLEIK |
| SEQ ID NO: 101 | DIVMTQSPATLSLSPGERATLSCRASANIYSYLAWYQQKPGQAPRFLVY |
| 7A3 | NAITLAEGVPARFSGSGSGTDFTLTISSLEPEDFAVYYCQHHYGTPFTFG |
| hu-VL3 | GGTKLEIK |
| SEQ ID NO: 102 | EIVMTQSPATLSLSPGERATLSCRASANIYSYLAWYQQKPGQAPRFLVY |
| 7A3 | NAITRATGIPARFSGSGSGTDFTLTISSLEPEDFAVYYCQHHYGTPFTFG |
| hu-VL4 | GGTKLEIK |
| SEQ ID NO: 103 | EVQLVQSGAEVKKPGASVKVSCKASGYSFTGYTMHWVRQAPGQGLE |
| 7B1 | WIGLINPYTGGTDYNQKFQGRVTLTVDRSTSTAYMELSSLRSEDTAVY |
| hu-VH1 | YCARERPGGSQFIYYYALDYWGQGTTVTVSS |
| SEQ ID NO: 104 | EVQLVQSGSELKKPGASVKVSCKASGYSFTGYTMHWVRQAPGQGLE |
| 7B1 | WIGLINPYTGGTDYNQGFTGRFVLSVDRSVSTAYLQISSLKAEDTAVYY |
| hu-VH3 | CARERPGGSQFIYYYALDYWGQGTTVTVSS |
| SEQ ID NO: 105 | QVQLVQSGSELKKPGASVKVSCKASGYSFTGYTMNWVRQAPGQGLE |
| 7B1 | WMGLINPYTGGTDYNQGFTGRFVFSVDRSVSTAYLQISSLKAEDTAVY |
| hu-VH4 | YCARERPGGSQFIYYYALDYWGQGTTVTVSS |
| SEQ ID NO: 106 | DIQMTQSPSSLSASVGDRVTITCRASENIYSYLAWYQQKPGKAPKFLVY |
| 7B1 | NADTLASGVPSRFSGSGSGTDFTLTISSLQPEDFATYYCQHHYGTPFTFG |
| hu-VL2 | GGTKLEIK |
| SEQ ID NO: 107 | DIVMTQSPATLSLSPGERATLSCRASENIYSYLAWYQQKPGQAPRFLVY |
| 7B1 | NADTLAEGVPARFSGSGSGTDFTLTISSLEPEDFAVYYCQHHYGTPFTF |
| hu-VL3 | GGGTKLEIK |
| SEQ ID NO: 108 | EIVMTQSPATLSLSPGERATLSCRASENIYSYLAWYQQKPGQAPRFLVY |
| 7B1 | NADTRATGIPARFSGSGSGTDFTLTISSLEPEDFAVYYCQHHYGTPFTFG |
| hu-VL4 | GGTKLEIK |
| SEQ ID NO: 109 | MGWSWIFLFLLSGTAGVHSEVQLQQSGPELAKPGASMKISCRASGYSF |
| 7A3 | TGYTIHWVKQSHGKNLEWIGLINPYTGGTDYNQKFKGKATLTVDKSSS |
| VH | TAYMDLLSLTSEDSAVYYCARERPGGSQFIYYYPMDYWGQGTSVTVSS |
| SEQ ID NO: 110 | MSVPTQVLGLLLLWLTGVRCDIQMTQSPASLSVSVGETVTITCRASANI |
| 7A3 | YSYLAWYQQKQGKSPQFLVYNAITLAEGVPSRFSGSGSGTQFSLKINSL |
| VL | QPEDFGSYYCQHHYGTPFTFGSGTKLEIK |
| SEQ ID NO: 111 | ATGGGATGGAGCTGGATCTTTCTCTTCCTCCTGTCAGGAACTGCAGG |
| 7A3 | TGTCCACTCTGAGGTCCAGCTGCAACAGTCTGGACCTGAGCTGGCG |
| VH | AAGCCTGGAGCTTCAATGAAGATATCCTGCAGGGCTTCTGGTTACTC |
| | ATTCACTGGCTACACCATCCACTGGGTGAAGCAGAGCCATGGAAAG |
| | AACCTTGAGTGGATTGGACTTATTAATCCTTACACTGGTGGTACTGA |
| | CTACAACCAGAAGTTCAAGGGCAAGGCCACATTAACTGTCGACAAG |
| | TCATCCAGCACAGCCTACATGGACCTCCTCAGTCTAACATCTGAAGA |
| | CTCTGCAGTCTACTACTGTGCAAGGGAGCGCCCCGGGGGGTCCCAA |
| | TTTATATATTATTATCCTATGGACTACTGGGGTCAAGGAACCTCGGT |
| | CACCGTCTCCTCA |
| SEQ ID NO: 112 | ATGAGTGTGCCCACTCAGGTCCTGGGGTTGCTGCTGCTGTGGCTTAC |
| 7A3 | AGGTGTCAGATGTGACATCCAGATGACTCAGTCTCCAGCCTCCCTAT |
| VL | CTGTATCTGTGGGAGAAACTGTCACCATCACATGTCGAGCAAGTGC |
| | GAATATTTACAGTTATTTAGCATGGTATCAGCAGAAACAGGGAAAA |
| | TCTCCTCAGTTCCTGGTCTATAATGCAATAACCTTAGCAGAAGGTGT |
| | GCCATCAAGGTTCAGTGGCAGTGGATCAGGCACACAGTTTTCTCTGA |
| | AGATCAACAGCCTGCAGCCTGAAGATTTTGGGAGTTATTACTGTCAA |
| | CATCATTATGGTACTCCATTCACGTTCGGCTCGGGGACAAAGTTGGA |
| | GATAAAA |
| SEQ ID NO: 113 | MGWSWIFLFLLSGTAGVHSEVQLQQSGPELAKPGASMKISCKASGYSF |
| 7B1 | TGYTMHWVKQSHGKNLEWIGLINPYTGGTDYNQKFKGKATLTVDRSS |
| VH | STAYMDLLSLTSEDSAVYYCARERPGGSQFIYYYALDYWGQGTSVTVS |
| | S |
| SEQ ID NO: 114 | MSVPTQVLGLLLLWLTGARCDIQMTQSPASLSVSVGETVTITCRASENI |
| 7B1 | YSYLAWYQQKQGRSPQFLVYNADTLAEGVPSRFSGSGSGTQFSLKINS |
| VL | LQPEDFGSYYCQHHYGTPFTFGSGTKLEIK |
| SEQ ID NO: 115 | ATGGGATGGAGCTGGATCTTTCTCTTCCTCCTGTCAGGAACTGCAGG |
| 7B1 | TGTCCACTCTGAGGTCCAGCTGCAACAGTCTGGACCTGAGCTGGCG |
| VH | AAGCCTGGAGCTTCAATGAAGATATCCTGTAAGGCTTCTGGTTACTC |
| | ATTCACTGGCTACACCATGCACTGGGTGAAGCAGAGCCATGGAAAG |
| | AACCTTGAGTGGATTGGACTAATTAATCCTTACACTGGTGGTACTGA |
| | CTACAACCAGAAGTTCAAGGGCAAGGCCACATTAACTGTCGACAGG |
| | TCTTCCAGCACAGCCTACATGGACCTCCTCAGTCTGACATCTGAGGA |
| | CTCTGCAGTCTATTACTGTGCAAGGGAGCGCCCCGGGGGGTCCCAA |
| | TTTATATATTACTATGCTCTGGACTACTGGGGTCAAGGAACCTCGGT |
| | CACCGTCTCCTCA |
| SEQ ID NO: 116 | ATGAGTGTGCCCACTCAGGTCCTGGGGTTGCTGCTGCTGTGGCTTAC |
| 7B1 | AGGTGCCAGATGTGACATCCAGATGACTCAGTCTCCAGCCTCCCTAT |
| VL | CTGTATCTGTGGGAGAAACTGTCACCATCACATGTCGAGCAAGTGA |
| | GAATATTTACAGTTATTTAGCATGGTATCAGCAGAAACAGGGAAGA |
| | TCTCCTCAGTTCCTGGTCTATAATGCAGATACCTTAGCGGAAGGTGT |
| | GCCATCAAGGTTCAGTGGCAGTGGATCAGGCACACAGTTTTCTCTAA |
| | AGATCAACAGCCTACAGCCTGAAGATTTTGGGAGTTATTACTGTCAA |
| | CATCATTATGGTACTCCATTCACGTTCGGCTCGGGGACAAAGTTGGA |
| | GATAAAA |

PD-L1 NA Sequences

| | |
|---|---|
| SEQ ID NO: 117 | ATGGAATTGGGGCTGAGCTGNGTTTTCCTTGTTGCTATTTTAGAAGG |
| 7H7 V_{H} DNA | TGTCCAGTGTGAGGTGCAACTGGTGGAGTCTGGGGGAGGCTTGGTC |
| sequence-with | CAGCCTGGGGGGTCCCTGAGACTCTCCTGTGCAGCCTCTGGAGGCA |
| signal sequence | CCATTAGTACCTCTTGGATGAGCTGGGTCCGCCAGGCTCCAGGGAA |
| | GGGGCTGGAATGGGTGGCCAACATAAAGCAAGATGGAAGTGAGAA |
| | ATATTATGTGGACTCTGTGAAGGGCCGATTCACCATCTCCAGAGACA |
| | ACGCCAAGAACTCACTGTATTTGCAAATGAACAGCCTGAGAGTCGA |
| | GGACACGGCTATATATTACTGTGCGAGAGATCGTCCAGTGGCTGGT |
| | GCGTCGGCCCTCTGGGGCCAGGGAACCCTGGTCACCGTCTCCTCA |
| SEQ ID NO: 118 | ATGAGGGTCCCCGCTCAGCTCCTGGGGCTCCTGCTGCTCTGGCTCCC |
| 7H7 V_{L} DNA | AGGTGCCAAATGTGACATCCAGATGACCCAGTCTCCTTCCACCCTGT |
| sequence-with | CTGCATCTGTAGGAGACAGAGTCACCATCACTTGCCGGGCCAGTCA |
| signal sequence | GAGTATAAGTGGCTGGTTGGCCTGGTATCAGCAGAAACAAGGGAAA |
| | GCCCCTAAGCTCCTGATCTATAAGGCGTCTAGTTTAGAAAGTGGGGT |
| | CCCATCAAGGTTCAGCGGCAGTGGATCTGGGACAGAATTCACTCTC |
| | ACCATCAGCAGCCTGCAGCCTGATGATTTTGCAACTTATTACTGCCA |
| | ACAGTATTATGGTTCTTCTCGGACGTTCGGCCAAGGGACCAATGTGG |
| | AAATCAAA |
| SEQ ID NO: 119 | ATGGAATTGGGGCTGAGCTGGGTTTTCCTTGTTGCTATTTTAGAAGG |
| 1B3 V_{H} DNA | TGTCCAGTGTGAGGTGCAACTGGTGGAGTCTGGGGGAGGCTTGGTC |
| sequence-with | CAGCCTGGGGGGTCCCTGAGACTCTCCTGTGCAGCCTCTGGAGGCA |
| signal sequence | CCATTAGTACCTCTTGGATGAGCTGGGTCCGCCAGGCTCCAGGGAA |
| | GGGGCTGGAATGGGTGGCCAACATAAAGCAAGATGGAAGTGAGAA |
| | ATATTATGTGGACTCTGTGAAGGGCCGATTCACCATCTCCAGAGACA |
| | ACGCCAAGAACTCACTGTATTTGCAAATGAACAGCCTGAGAGTCGA |
| | AGACACGGCTATGTATTACTGTGCGAGAGATCGTCCAGTGGCTGGT |
| | GCGTCGGCCCTCTGGGGCCAGGGAACCCTGGTCACCGTCTCCTCA |
| SEQ ID NO: 120 | ATGAGGGTCCCCGCTCAGCTCCTGGGGCTCCTGCTGCTCTGGCTCCC |
| 1B3 V_{L} DNA | AGGTGCCAAATGTGACATCCAGATGACCCAGTCTCCTTCCACCCTGT |
| sequence-with | CTGCATCTGTAGGAGACAGAGTCACCATCACTTGCCGGGCCAGTCA |
| signal sequence | GAGTATTAGTGGCTGGTTGGCCTGGTATCAGCAGAAACCAGGGAAA |
| | GCCCCTAAGCTCCTGATCTATAAGGCGTCTAGTTTAGAAAGTGGGGT |
| | CCCATCAAGGTTCAGCGGCAGTGGATCTGGGACAGAATTCACTCTC |
| | ACCATCAGCAGCCTGCAGCCTGATGATTTTGCAACTTATTACTGCCA |
| | ACAGTATTATGGTTCTTCTCGGACGTTCGGCCAAGGGACCAATGTGG |
| | AAATCAAA |
| SEQ ID NO: 121 | ATGGAATTGGGGCTGAGCTGGGTTTTCCTTGTTGCTATTTTAGAAGG |
| 3B6 V_{H} DNA | TGTCCAGTGTGAGGTGCAACTGGTGGAGTCTGGGGGAGGCTTGGTC |
| sequence-with | CAGCCTGGGGGGTCCCTGAGACTCTCCTGTGCAGCCTCTGGAGGCA |
| signal sequence | CAACCAGTACCTATTGGATGAGCTGGGTCCGCCAGGCTCCAGGGAA |
| | GGGGCTGGAATGGGTGGCCAACATAAAGCAAGATGGAAGTGAGAA |
| | ATATTATGTGGACTCTGTGAAGGGCCGATTCACCATCTCCAGAGACA |
| | ACGCCAAGAACTCACTGAATTTGCAAATGAACAGCCTGAGAGTCGA |
| | GGACACGGCTATATATTACTGTGCGAGAGATCGTCCAGTGGCTGGT |
| | GCGTCGGCCCTCTGGGGCCAGGGAACCCTGGTCACCGTCTCCTCA |
| SEQ ID NO: 122 | ATGAGGGTCCCCGCTCAGCTCCTGGGGCTCCTGCTGCTCTGGCTCCC |
| 3B6 V_{L} DNA | AGGTGCCAAATGTGACATCCAGATGACCCAGTCTCCTTCCACCCTGT |
| sequence-with | CTGCATCTGTAGGAGACAGAGTCACCATCACTTGCCGGGCCAGTCA |
| signal sequence | GAGTATTAGTGGCTGGTTGGCCTGGTATCAGCAGAAACCAGGGAAA |
| | GCCCCTAAGCTCCTGATCTATAAGGCGTCTAGTTTAGAAAGTGGGGT |
| | CCCATCAAGGTTCAGCGGCAGTGGATCTGGGACAGAATTCACTCTC |
| | ACCATCAGCAGCCTGCAGCCTGATGATTTTGCAACTTATTACTGCCA |
| | ACAGTATTATGGTTCTTCTCGGACGTTCGGCCAAGGGACCAATGTGG |
| | AAATCAAA |
| SEQ ID NO: 123 | ATGGAATTGGGGCTGAGCTGGGTTTTCCTTGTTGCTATTTTAGAAGG |
| 8B1 V_{H} DNA | TGTCAAGTGTGAGGTGCGACTGGTGGAGTCTGGGGGAGGCTTGGTC |
| sequence-with | CAGCCTGGGGGGTCCCTGAGACTCTCCTGTGCAGCCTCTGGAGACAT |
| signal sequence | AATTAGTACCCATTGGATGAGCTGGGTCCGCCAGGCTCCAGGGAAG |
| | GGGCTGGAATGGGTGGCCAACATAAAACAAGATGGAAGTGAGAAG |
| | TATTATGTGGACTCTGTGAAGGGCCGATTCACCATCTCCAGAGACAA |
| | CGCCAAGAACTCACTGTATTTGCAAATGAACACCCTGAGAGTCGAG |
| | GACACGGCTATATATTACTGTACGAGAGATCGTCCAGTGGCTGGTG |
| | CGTCGGCCCTCTGGGGCCAGGGAACCCTGGTCACCGTCTCCTCA |
| SEQ ID NO: 124 | ATGAGGGTCCCCGCTCAGCTCCTGGGGCTCCTGCTGCTCTGGCTCCC |
| 8B1 V_{L} DNA | AGGTGCCAAATGTGACATCCAGATGACCCAGTCTCCTTCCACCCTGT |
| sequence-with | CTGCATCTGTAGGAGACAGAGTCACCATCACTTGCCGGGCCAGTCA |
| signal sequence | GAGTATTAGTGGCTGGTTGGCCTGGTATCAGCAGAAACCAGGGAAA |
| | GCCCCTAAGCTCCTGATCTATAAGGCGTCTAGTTTAGAAAGTGGGGT |
| | CCCATTAAGGTTCAGCGGCAGTGGATCTGGGACAGAATTCACTCTC |
| | ACCATCAGCAGCCTGCAGCCTGATGATTTTGCAACTTATTACTGCCA |
| | ACAGTATTATGGTTCTTCTCGGACGTTCGGCCAAGGGACCAATGTGG |
| | AAATCAAA |
| SEQ ID NO: 125 | ATGGAATTGGGGCTGAGCTGGGTTTTCCTTGTTGCTATTTTAGAAGG |
| 4A3 V_{H} DNA | TGTCCAGTGTGAGGTGCAACTGGTGGAGTCTGGGGGAGGCTTGGTC |
| sequence-with | CAGCCGGGGGGGTCCCTGAGACTCTCCTGTGCAGCCTCTGGAGGCA |
| signal sequence | TCATTAGTTCCTCTTGGATGAGCTGGGTCCGCCAGGCTCCAGGGAAG |
| | GGGCTGGAATGGGTGGCCAACATAAAGCAAGATGGAAGTGAGAAA |
| | TATTATGTGGACTCTGTGAAGGGCCGATTCACCATCTCCAGAGACAA |
| | CGCCAAAGACTTACTGTATTTGCAAATGAACAGCCTGAGAGTCGAG |
| | GACACGGCTTTATATTACTGTGCGAGAGATCGTCCAGTGGCTGGTGC |
| | GTCGGCCCTCTGGGGCCAGGGAACCCTGGTCACCGTCTCCTCT |
| SEQ ID NO: 126 | ATGAGGGTCCCCGCTCAGCTCCTGGGGCTCCTGCTGCTCTGGCTCCC |
| 4A3 V_{L} DNA | AGGTGCCAAATGTGACATCCAGATGACCCAGTCTCCTTCCACCCTGT |
| sequence-with | CTGCATCTGTAGGAGACAGAGTCACCATCACTTGCCGGGCCAGTCA |
| signal sequence | GAGTATTAGTGGCTGGTTGGCCTGGTATCAGCAGAAACCAGGGAAA |
| | GCCCCTAAGCTCCTGATCTATAAGGCGTCTAGTTTAGAAAGTGGGGT |
| | CCCATCAAGGTTCAGCGGCAGTGGATCTGGGACAGAATTCACTCTC |
| | ACCATCAGCAGCCTGCAGCCTGATGATTTTGCAACTTATTACTGCCA |
| | ACAGTATTATGGTTCTTCTCGGACGTTCGGCCAAGGGACCAATGTGG |
| | AAATCAAA |
| SEQ ID NO: 127 | ATGGAATTGGGGCTGAGCTGGGTTTTCCTTGTTGCTATTTTAGAAGG |
| 9H9 V_{H} DNA | TGTCCAGTGTGAGGTGCAACTGGTGGAGTCTGGGGGAGGCTTGGTC |
| sequence-with | CAGCCTGGGGGGTCCCTGAGACTCTCCTGTGCAGCCTCTGGAGGCAT |
| signal sequence | CATTAGTACCTATTGGATGAGCTGGGTCCGCCAGGCTCCAGGGAAG |
| | GGGCTGGAATGGGTGGCCAACATAAAGCAAGATGGAAGTGAGAAA |
| | TATTATGTGGACTCTGTGAAGGGCCGATTCACCATCTCCAGAGACAA |
| | CGCCAAGAACTCACTGTATTTGCAAATGAACAGCCTGAGAGTCGAG |
| | GACACGGCTATGTATTACTGTGCGAGAGATCGTCCAGTGGCTGGTG |
| | CGTCGGCCCTCTGGGGCCAGGGAACCCTGGTCACCGTCTCCTCA |
| SEQ ID NO: 128 | ATGAGGGTCCCCGCTCAGCTCCTGGGGCTCCTGCTGCTCTGGCTCCC |
| 9H9 V_{L} DNA | AGGTGCCAAATGTGACATCCAGATGACCCAGTCTCCTTCCACCCTGT |
| sequence-with | CTGCATCTGTAGGAGACAGAGTCACCATCACTTGCCGGGCCAGTCA |
| signal sequence | GAGTATTAGTGGCTGGTTGGCCTGGTATCAGCAGAAACCAGGGAAA |
| | GCCCCTAAGCTCCTGATCTATAAGGCGTCTAGTTTAGAAAGTGGGGT |
| | CCCATCAAGGTTCAGCGGCAGTGGATCTGGGACAGAATTCACTCTC |
| | ACCATCAGCAGCCTGCAGCCTGATGATTTTGCAACTTATTACTGCCA |
| | ACAGTATTATGGTTCTTCTCGGACGTTCGGCCAAGGGACCAATGTGG |
| | AAATCAAA |
| SEQ ID NO: 129 | GAGGTGCAACTGGTGGAGTCTGGGGGAGGCTTGGTCCAGCCTGGGG |
| 7H7 V_{H} DNA | GGTCCCTGAGACTCTCCTGTGCAGCCTCTGGAGGCACCATTAGTACC |
| sequence- | TCTTGGATGAGCTGGGTCCGCCAGGCTCCAGGGAAGGGGCTGGAAT |
| without signal | GGGTGGCCAACATAAAGCAAGATGGAAGTGAGAAATATTATGTGGA |
| sequence | CTCTGTGAAGGGCCGATTCACCATCTCCAGAGACAACGCCAAGAAC |
| | TCACTGTATTTGCAAATGAACAGCCTGAGAGTCGAGGACACGGCTA |
| | TATATTACTGTGCGAGAGATCGTCCAGTGGCTGGTGCGTCGGCCCTC |
| | TGGGGCCAGGGAACCCTGGTCACCGTCTCCTCA |
| SEQ ID NO: 130 | GACATCCAGATGACCCAGTCTCCTTCCACCCTGTCTGCATCTGTAGG |
| 7H7 V_{L} DNA | AGACAGAGTCACCATCACTTGCCGGGCCAGTCAGAGTATAAGTGGC |
| sequence- | TGGTTGGCCTGGTATCAGCAGAAACAAGGGAAAGCCCCTAAGCTCC |
| without signal | TGATCTATAAGGCGTCTAGTTTAGAAAGTGGGGTCCCATCAAGGTTC |
| sequence | AGCGGCAGTGGATCTGGGACAGAATTCACTCTCACCATCAGCAGCC |
| | TGCAGCCTGATGATTTTGCAACTTATTACTGCCAACAGTATTATGGT |
| | TCTTCTCGGACGTTCGGCCAAGGGACCAATGTGGAAATCAAA |
| SEQ ID NO: 131 | GAGGTGCAACTGGTGGAGTCTGGGGGAGGCTTGGTCCAGCCTGGGG |
| 1B3 V_{H} DNA | GGTCCCTGAGACTCTCCTGTGCAGCCTCTGGAGGCACCATTAGTACC |
| sequence- | TCTTGGATGAGCTGGGTCCGCCAGGCTCCAGGGAAGGGGCTGGAAT |
| without signal | GGGTGGCCAACATAAAGCAAGATGGAAGTGAGAAATATTATGTGGA |
| sequence | CTCTGTGAAGGGCCGATTCACCATCTCCAGAGACAACGCCAAGAAC |
| | TCACTGTATTTGCAAATGAACAGCCTGAGAGTCGAAGACACGGCTA |
| | TGTATTACTGTGCGAGAGATCGTCCAGTGGCTGGTGCGTCGGCCCTC |
| | TGGGGCCAGGGAACCCTGGTCACCGTCTCCTCA |
| SEQ ID NO: 132 | GACATCCAGATGACCCAGTCTCCTTCCACCCTGTCTGCATCTGTAGG |
| 1B3 V_{L} DNA | AGACAGAGTCACCATCACTTGCCGGGCCAGTCAGAGTATTAGTGGC |
| sequence- | TGGTTGGCCTGGTATCAGCAGAAACCAGGGAAAGCCCCTAAGCTCC |
| without signal | TGATCTATAAGGCGTCTAGTTTAGAAAGTGGGGTCCCATCAAGGTTC |
| sequence | AGCGGCAGTGGATCTGGGACAGAATTCACTCTCACCATCAGCAGCC |
| | TGCAGCCTGATGATTTTGCAACTTATTACTGCCAACAGTATTATGGT |
| | TCTTCTCGGACGTTCGGCCAAGGGACCAATGTGGAAATCAAA |
| SEQ ID NO: 133 | GAGGTGCAACTGGTGGAGTCTGGGGGAGGCTTGGTCCAGCCTGGGG |
| 3B6 V_{H} DNA | GGTCCCTGAGACTCTCCTGTGCAGCCTCTGGAGGCACAACCAGTACC |
| sequence- | TATTGGATGAGCTGGGTCCGCCAGGCTCCAGGGAAGGGGCTGGAAT |
| without signal | GGGTGGCCAACATAAAGCAAGATGGAAGTGAGAAATATTATGTGGA |
| sequence | CTCTGTGAAGGGCCGATTCACCATCTCCAGAGACAACGCCAAGAAC |
| | TCACTGAATTTGCAAATGAACAGCCTGAGAGTCGAGGACACGGCTA |
| | TATATTACTGTGCGAGAGATCGTCCAGTGGCTGGTGCGTCGGCCCTC |
| | TGGGGCCAGGGAACCCTGGTCACCGTCTCCTCA |
| SEQ ID NO: 134 | GACATCCAGATGACCCAGTCTCCTTCCACCCTGTCTGCATCTGTAGG |
| 3B6 V_{L} DNA | AGACAGAGTCACCATCACTTGCCGGGCCAGTCAGAGTATTAGTGGC |
| sequence- | TGGTTGGCCTGGTATCAGCAGAAACCAGGGAAAGCCCCTAAGCTCC |
| without signal | TGATCTATAAGGCGTCTAGTTTAGAAAGTGGGGTCCCATCAAGGTTC |
| sequence | AGCGGCAGTGGATCTGGGACAGAATTCACTCTCACCATCAGCAGCC |
| | TGCAGCCTGATGATTTTGCAACTTATTACTGCCAACAGTATTATGGT |
| | TCTTCTCGGACGTTCGGCCAAGGGACCAATGTGGAAATCAAA |
| SEQ ID NO: 135 | GAGGTGCGACTGGTGGAGTCTGGGGGAGGCTTGGTCCAGCCTGGGG |
| 8B1 V_{H} DNA | GGTCCCTGAGACTCTCCTGTGCAGCCTCTGGAGACATAATTAGTACC |
| sequence- | CATTGGATGAGCTGGGTCCGCCAGGCTCCAGGGAAGGGGCTGGAAT |
| without signal | GGGTGGCCAACATAAAACAAGATGGAAGTGAGAAGTATTATGTGGA |
| sequence | CTCTGTGAAGGGCCGATTCACCATCTCCAGAGACAACGCCAAGAAC |
| | TCACTGTATTTGCAAATGAACACCCTGAGAGTCGAGGACACGGCTA |
| | TATATTACTGTACGAGAGATCGTCCAGTGGCTGGTGCGTCGGCCCTC |
| | TGGGGCCAGGGAACCCTGGTCACCGTCTCCTCA |
| SEQ ID NO: 136 | GACATCCAGATGACCCAGTCTCCTTCCACCCTGTCTGCATCTGTAGG |
| 8B1 V_{L} DNA | AGACAGAGTCACCATCACTTGCCGGGCCAGTCAGAGTATTAGTGGC |
| sequence- | TGGTTGGCCTGGTATCAGCAGAAACCAGGGAAAGCCCCTAAGCTCC |
| without signal | TGATCTATAAGGCGTCTAGTTTAGAAAGTGGGGTCCCATTAAGGTTC |
| sequence | AGCGGCAGTGGATCTGGGACAGAATTCACTCTCACCATCAGCAGCC |
| | TGCAGCCTGATGATTTTGCAACTTATTACTGCCAACAGTATTATGGT |
| | TCTTCTCGGACGTTCGGCCAAGGGACCAATGTGGAAATCAAA |
| SEQ ID NO: 137 | GAGGTGCAACTGGTGGAGTCTGGGGGAGGCTTGGTCCAGCCGGGGG |
| 4A3 V_{H} DNA | GGTCCCTGAGACTCTCCTGTGCAGCCTCTGGAGGCATCATTAGTTCC |
| sequence- | TCTTGGATGAGCTGGGTCCGCCAGGCTCCAGGGAAGGGGCTGGAAT |
| without signal | GGGTGGCCAACATAAAGCAAGATGGAAGTGAGAAATATTATGTGGA |
| sequence | CTCTGTGAAGGGCCGATTCACCATCTCCAGAGACAACGCCAAAGAC |
| | TTACTGTATTTGCAAATGAACAGCCTGAGAGTCGAGGACACGGCTTT |
| | ATATTACTGTGCGAGAGATCGTCCAGTGGCTGGTGCGTCGGCCCTCT |
| | GGGGCCAGGGAACCCTGGTCACCGTCTCCTCT |
| SEQ ID NO: 138 | GACATCCAGATGACCCAGTCTCCTTCCACCCTGTCTGCATCTGTAGG |
| 4A3 V_{L} DNA | AGACAGAGTCACCATCACTTGCCGGGCCAGTCAGAGTATTAGTGGC |
| sequence- | TGGTTGGCCTGGTATCAGCAGAAACCAGGGAAAGCCCCTAAGCTCC |
| without signal | TGATCTATAAGGCGTCTAGTTTAGAAAGTGGGGTCCCATCAAGGTTC |
| sequence | AGCGGCAGTGGATCTGGGACAGAATTCACTCTCACCATCAGCAGCC |
| | TGCAGCCTGATGATTTTGCAACTTATTACTGCCAACAGTATTATGGT |
| | TCTTCTCGGACGTTCGGCCAAGGGACCAATGTGGAAATCAAA |
| SEQ ID NO: 139 | GAGGTGCAACTGGTGGAGTCTGGGGGAGGCTTGGTCCAGCCTGGGG |
| 9H9 V_{H} DNA | GGTCCCTGAGACTCTCCTGTGCAGCCTCTGGAGGCATCATTAGTACC |
| sequence- | TATTGGATGAGCTGGGTCCGCCAGGCTCCAGGGAAGGGGCTGGAAT |
| without signal | GGGTGGCCAACATAAAGCAAGATGGAAGTGAGAAATATTATGTGGA |
| sequence | CTCTGTGAAGGGCCGATTCACCATCTCCAGAGACAACGCCAAGAAC |
| | TCACTGTATTTGCAAATGAACAGCCTGAGAGTCGAGGACACGGCTA |
| | TGTATTACTGTGCGAGAGATCGTCCAGTGGCTGGTGCGTCGGCCCTC |
| | TGGGGCCAGGGAACCCTGGTCACCGTCTCCTCA |
| SEQ ID NO: 140 | GACATCCAGATGACCCAGTCTCCTTCCACCCTGTCTGCATCTGTAGG |
| 9H9 V_{L} DNA | AGACAGAGTCACCATCACTTGCCGGGCCAGTCAGAGTATTAGTGGC |
| sequence- | TGGTTGGCCTGGTATCAGCAGAAACCAGGGAAAGCCCCTAAGCTCC |
| without signal | TGATCTATAAGGCGTCTAGTTTAGAAAGTGGGGTCCCATCAAGGTTC |
| sequence | AGCGGCAGTGGATCTGGGACAGAATTCACTCTCACCATCAGCAGCC |
| | TGCAGCCTGATGATTTTGCAACTTATTACTGCCAACAGTATTATGGT |
| | TCTTCTCGGACGTTCGGCCAAGGGACCAATGTGGAAATCAAA |

Bispecific Antibody Sequences
Key for SEQ ID NOs:141-145
Bold double underlined: 9H9 Variable region
Bold single underlined: Constant domains (including embedded dotted underline)
Italic: anti-ILT4 scFv
Italic underlined: linkers
Dotted Underline: modified amino acid residues

| | |
|---|---|
| SEQ ID NO: 141 | EVQLVESGGGLVQPGGSLRLSCAASGGIISTYWMSWVRQAPGKGLEWVANI |
| 9H9-7A3 HL | KQDGSEKYYVDSVKGRFTISRDNAKNSLYLQMNSLRVEDTAMYYCARDRP |
| | VAGASALWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKKVEPKSCDKTHTCPPCPAPE [symbol] GGPSVFLFPPKPKDTLMIS |
| | RTPEVTCVVVDVSHEDPEVKFNWYVDGVEVHNAKTKPREEQYNSTYRVVS |
| | VLTVLHQDWLNGKEYKCQVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGSSGGGGSQV |
| | QLVQSGAEVKKPGASVKVSCKASGYSFTGYTMHWVRQAPGQ [symbol] LEWMGLINPYTG |
| | GTDYNQKFQGRVTMTVDKSTSTAYMELSSLRSEDTAVYYCARERPGGSQFIYYYP |
| | MDYWGQGTTVTVSSGGGGSGGGGSGGGGSGGGGSDIQMTQSPSSLSASVGDRVTIT |
| | CRASANIYSYLAWYQQKPGKAPKFLVYNAITLAEGVPSRFSGSGSGTDFTLTISSLQ |
| | PEDFATYYCQHHYGTPFTFG [symbol] GTKLEIK |
| SEQ ID NO: 142 | EVQLVESGGGLVQPGGSLRLSCAASGGIISTYWMSWVRQAPGKGLEWVANI |
| 9H9-7A3 LH | KQDGSEKYYVDSVKGRFTISRDNAKNSLYLQMNSLRVEDTAMYYCARDRP |
| | VAGASALWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKKVEPKSCDKTHTCPPCPAPE [symbol] GGPSVFLFPPKPKDTLMIS |
| | RTPEVTCVVVDVSHEDPEVKFNWYVDGVEVHNAKTKPREEQYNSTYRVVS |
| | VLTVLHQDWLNGKEYKCQVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGSSGGGGSDIQ |
| | MTQSPSSLSASVGDRVTITCRASANIYSYLAWYQQKPGKAPKFLVYNAITLAEGVPS |
| | RFSGSGSGTDFTLTISSLQPEDFATYYCQHHYGTPFTFG [symbol] GTKLEIKGGGGSGGGGS |
| | GGGGSGGGGSQVQLVQSGAEVKKPGASVKVSCKASGYSFTGYTMHWVRQAPGQ [symbol] L |
| | EWMGLINPYTGGTDYNQKFQGRVTMTVDKSTSTAYMELSSLRSEDTAVYYCARE |
| | RPGGSQFIYYYPMDYWGQGTTVTVSS |
| SEQ ID NO: 143 | EVQLVESGGGLVQPGGSLRLSCAASGGIISTYWMSWVRQAPGKGLEWVANI |
| 9H9-7B1 HL | KQDGSEKYYVDSVKGRFTISRDNAKNSLYLQMNSLRVEDTAMYYCARDRP |
| | VAGASALWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKKVEPKSCDKTHTCPPCPAPE [symbol] GGPSVFLFPPKPKDTLMIS |
| | RTPEVTCVVVDVSHEDPEVKFNWYVDGVEVHNAKTKPREEQYNSTYRVVS |
| | VLTVLHQDWLNGKEYKCQVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGSSGGGGSQV |
| | QLVQSGAEVKKPGASVKVSCKASGYSFTGYTMHWVRQAPGQ [symbol] LEWMGLINPYTG |
| | GTDYNQKFQGRVTMTVDRSTSTAYMELSSLRSEDTAVYYCARERPGGSQFIYYYAL |
| | DYWGQGTTVTVSSGGGGSGGGGSGGGGSGGGGSDIQMTQSPSSLSASVGDRVTITC |
| | RASENIYSYLAWYQQKPGKAPKFLVYNADTLAEGVPSRFSGSGSGTDFTLTISSLQP |
| | EDFATYYCQHHYGTPFTFG [symbol] GTKLEIK |
| SEQ ID NO: 144 | EVQLVESGGGLVQPGGSLRLSCAASGGIISTYWMSWVRQAPGKGLEWVANI |
| 9H9-7B1 LH | KQDGSEKYYVDSVKGRFTISRDNAKNSLYLQMNSLRVEDTAMYYCARDRP |
| | VAGASALWGQGTLVTVSSASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKKVEPKSCDKTHTCPPCPAPE [symbol] GGPSVFLFPPKPKDTLMIS |
| | RTPEVTCVVVDVSHEDPEVKFNWYVDGVEVHNAKTKPREEQYNSTYRVVS |
| | VLTVLHQDWLNGKEYKCQVSNKALPAPIEKTISKAKGQPREPQVYTLPPSR |
| | DELTKNQVSLTCLVKGFYPSDIAVEWESNGQPENNYKTTPPVLDSDGSFFL |
| | YSKLTVDKSRWQQGNVFSCSVMHEALHNHYTQKSLSLSPGKGSSGGGGSDIQ |
| | MTQSPSSLSASVGDRVTITCRASENIYSYLAWYQQKPGKAPKFLVYNADTLAEGVP |
| | SRFSGSGSGTDFTLTISSLQPEDFATYYCQHHYGTPFTFG [symbol] GTKLEIKGGGGGGGG |
| | SGGGGSGGGGSQVQLVQSGAEVKKPGASVKVSCKASGYSFTGYTMHWVRQAPGQ [symbol] |
| | LEWMGLINPYTGGTDYNQKFQGRVTMTVDRSTSTAYMELSSLRSEDTAVYYCAR |
| | ERPGGSQFIYYYALDYWGQGTTVTVSS |
| SEQ ID NO: 145 | DIQMTQSPSTLSASVGDRVTITCRASQSISGWLAWYQQKPGKAPKLLIYKASSLE |
| 9H9 (light chain) | SGVPSRESGSGSGTEFTLTISSLQPDDFATYYCQQYYGSSRTFGQGTNVEIK |
| | RTVAAPSVFIFPPSDEQLKSGTASVVCLLNNFYPREAKVQWKVDNALQSGNSQE |
| | SVTEQDSKDSTYSLSSTLTLSKADYEKHKVYACEVTHQGLSSPVTKSENRGEC |

Key for SEQ ID NOs: 146-
Double underlined: 9H9 Variable region
Single underlined: Constant domains (including embedded dotted underline)
Italics: scFv
Italic underlined: Linkers
Dotted underline: modified bases

| | |
|---|---|
| SEQ ID NO: 146 | GAGGTGCAACTGGTGGAGTCTGGGGGAGGCTTGGTCCAGCCTGGGGGGTCCCTGA |
| 9H9 AQQ-7A3 | GACTCTCCTGTGCAGCCTCTGGAGGCATCATTAGTACCTATTGGATGAGCTGGGTCC |
| H2L1 | GCCAGGCTCCAGGGAAGGGGCTGGAATGGGTGGCCAACATAAAGCAAGATGGAAG |
| | TGAGAAATATTATGTGGACTCTGTGAAGGGCCGATTCACCATCTCCAGAGACAACGC |
| | CAAGAACTCACTGTATTTGCAAATGAACAGCCTGAGAGTCGAGGACACGGCTATGT |
| | ATTACTGTGCGAGAGATCGTCCAGTGGCTGGTGCGTCGGCCCTCTGGGGCCAGGGA |
| | ACCCTGGTCACCGTCTCCTCAGCTAGCACAAAGGGCCCTTCCGTGTTTCCACTGGCTC |
| | CCAGCTCTAAGTCCACCAGCGGAGGAACAGCCGCTCTGGGCTGTCTGGTGAAGGAC |
| | TATTTCCCAGAGCCCGTGACCGTGAGCTGGAACTCTGGCGCCCTGACCAGCGGAGT |
| | GCATACATTTCCTGCTGTGCTGCAGTCCAGCGGCCTGTACTCTCTGTCTTCCGTGGTG |
| | ACCGTGCCAAGCTCTTCCCTGGGCACCCAGACATATATCTGCAACGTGAATCACAAG |
| | CCATCCAATACAAAGGTGGACAAGAAGGTGGAGCCCAAGAGCTGTGATAAGACCCA |
| | TACATGCCCCCCTTGTCCTGCTCCAGAG [symbol] GGAGGACCATCCGTGTTCCTGTTT |
| | CCACCCAAGCCTAAGGACACCCTGATGATCTCTAGGACCCCAGAGGTGACCTGCGTG |
| | GTGGTGGACGTGAGCCACGAGGATCCCGAGGTGAAGTTCAACTGGTACGTGGATG |
| | GCGTGGAGGTGCATAATGCCAAGACAAAGCCAAGGGAGGAGCAGTACAATAGCAC |
| | CTATCGGGTGGTGTCTGTGCTGACAGTGCTGCACCAGGACTGGCTGAACGGCAAGG |
| | AGTACAAGTGC [symbol] GTGTCTAATAAGGCCCTGCCCGCTCCTATCGAGAAGACCATCT |
| | CCAAGGCCAAGGGCCAGCCTAGGGAGCCACAGGTGTACACACTGCCTCCAAGCCGG |
| | GACGAGCTGACCAAGAACCAGGTGTCTCTGACATGTCTGGTGAAGGGCTTCTATCCC |
| | TCTGATATCGCTGTGGAGTGGGAGTCCAATGGCCAGCCTGAGAACAATTACAAGAC |
| | CACACCCCCTGTGCTGGACTCCGATGGCAGCTTCTTTCTGTATTCCAAGCTGACCGTG |
| | GATAAGAGCAGGTGGCAGCAGGGCAACGTGTTTTCTTGTTCCGTGATGCATGAGGC |
| | TCTGCACAATCATTACACACAGAAGAGCCTGTCTCTGTCCCCTGGCAAAGGCTCGAG |
| | CGGGGGAGGAGGTAGCCAAGTGCAGCTGGTGCAGAGCGGCGCTGAGGTGAAGAA |
| | GCCCGGCGCCAGCGTCAAGGTCAGCTGTAAGGCCAGCGGCTACAGCTTCACTGGCTA |
| | CACTATGCACTGGGTGAGACAAGCCCCCGGCCAA [symbol] CTGGAGTGGATGGGACTGA |
| | TCAACCCTTACACTGGCGGCACTGACTACAACCAGAAATTCCAAGGAAGGGTCACAA |
| | TGACTGTGGACAAGAGCACATCCACTGCCTACATGGAGCTGAGCTCTCTGAGGAGCG |
| | AGGACACAGCCGTGTACTACTGTGCCAGAGAGAGACCCGGCGGCAGCCAGTTCATC |
| | TACTACTACCCTATGGACTACTGGGGCCAAGGCACAACAGTGACTGTGAGCAGCGG |
| | AGGGGGCGGTTCCGGAGGAGGCGGCAGCGGGGGAGGAGGTAGCGGCGGAGGTG |
| | GGTCTGACATCCAGATGACACAGAGCCCAAGCTCTCTGAGCGCCAGCGTGGGAGAT |
| | AGGGTGACAATCACATGTAGGGCCAGCGCCAACATCTACAGCTATCTGGCTTGGTAC |
| | CAGCAGAAGCCCGGCAAGGCCCCTAAGTTTCTGGTGTACAACGCCATCACTCTGGCT |
| | GAGGGCGTGCCTAGCAGATTTAGCGGCAGCGGAAGCGGCACAGACTTCACTCTGAC |
| | AATCAGCTCTCTGCAGCCAGAGGATTTCGCCACATACTACTGCCAGCACCACTACGGC |
| | ACTCCTTTCACATTCGGC [symbol] GGCACTAAGCTGGAGATCAAG |
| SEQ ID NO: 147 | GAGGTGCAACTGGTGGAGTCTGGGGGAGGCTTGGTCCAGCCTGGGGGGTCCCTGA |
| 9H9 AQQ-7A3 | GACTCTCCTGTGCAGCCTCTGGAGGCATCATTAGTACCTATTGGATGAGCTGGGTCC |
| L1H2 | GCCAGGCTCCAGGGAAGGGGCTGGAATGGGTGGCCAACATAAAGCAAGATGGAAG |
| | TGAGAAATATTATGTGGACTCTGTGAAGGGCCGATTCACCATCTCCAGAGACAACGC |
| | CAAGAACTCACTGTATTTGCAAATGAACAGCCTGAGAGTCGAGGACACGGCTATGT |
| | ATTACTGTGCGAGAGATCGTCCAGTGGCTGGTGCGTCGGCCCTCTGGGGCCAGGGA |
| | ACCCTGGTCACCGTCTCCTCA GCTAGCACAAAGGGCCCTTCCGTGTTTCCACTGGCTC |
| | CCAGCTCTAAGTCCACCAGCGGAGGAACAGCCGCTCTGGGCTGTCTGGTGAAGGAC |
| | TATTTCCCAGAGCCCGTGACCGTGAGCTGGAACTCTGGCGCCCTGACCAGCGGAGT |
| | GCATACATTTCCTGCTGTGCTGCAGTCCAGCGGCCTGTACTCTCTGTCTTCCGTGGTG |
| | ACCGTGCCAAGCTCTTCCCTGGGCACCCAGACATATATCTGCAACGTGAATCACAAG |
| | CCATCCAATACAAAGGTGGACAAGAAGGTGGAGCCCAAGAGCTGTGATAAGACCCA |
| | TACATGCCCCCCTTGTCCTGCTCCAGAG [symbol] GGAGGACCATCCGTGTTCCTGTTT |
| | CCACCCAAGCCTAAGGACACCCTGATGATCTCTAGGACCCCAGAGGTGACCTGCGTG |
| | GTGGTGGACGTGAGCCACGAGGATCCCGAGGTGAAGTTCAACTGGTACGTGGATG |
| | GCGTGGAGGTGCATAATGCCAAGACAAAGCCAAGGGAGGAGCAGTACAATAGCAC |
| | CTATCGGGTGGTGTCTGTGCTGACAGTGCTGCACCAGGACTGGCTGAACGGCAAGG |
| | AGTACAAGTGC [symbol] GTGTCTAATAAGGCCCTGCCCGCTCCTATCGAGAAGACCATCT |
| | CCAAGGCCAAGGGCCAGCCTAGGGAGCCACAGGTGTACACACTGCCTCCAAGCCGG |
| | GACGAGCTGACCAAGAACCAGGTGTCTCTGACATGTCTGGTGAAGGGCTTCTATCCC |
| | TCTGATATCGCTGTGGAGTGGGAGTCCAATGGCCAGCCTGAGAACAATTACAAGAC |
| | CACACCCCCTGTGCTGGACTCCGATGGCAGCTTCTTTCTGTATTCCAAGCTGACCGTG |
| | GATAAGAGCAGGTGGCAGCAGGGCAACGTGTTTTCTTGTTCCGTGATGCATGAGGC |
| | TCTGCACAATCATTACACACAGAAGAGCCTGTCTCTGTCCCCTGGCAAAGGCTCGAG |
| | CGGGGGAGGAGGTAGCGACATCCAGATGACACAGAGCCCAAGCTCTCTGAGCGCCA |
| | GCGTGGGAGATAGGGTGACAATCACATGTAGGGCCAGCGCCAACATCTACAGCTAT |
| | CTGGCTTGGTACCAGCAGAAGCCCGGCAAGGCCCCTAAGTTTCTGGTGTACAACGCC |
| | ATCACTCTGGCTGAGGGCGTGCCTAGCAGATTTAGCGGCAGCGGAAGCGGCACAGA |
| | CTTCACTCTGACAATCAGCTCTCTGCAGCCAGAGGATTTCGCCACATACTACTGCCAG |
| | CACCACTACGGCACTCCTTTCACATTCGGC [symbol] GGCACTAAGCTGGAGATCAAGGGA |
| | GGGGGCGGTTCCGGAGGAGGCGGCAGCGGGGGAGGAGGTAGCGGCGGAGGTGG |
| | GTCTCAAGTGCAGCTGGTGCAGAGCGGCGCTGAGGTGAAGAAGCCCGGCGCCAGC |
| | GTCAAGGTCAGCTGTAAGGCCAGCGGCTACAGCTTCACTGGCTACACTATGCACTGG |
| | GTGAGACAAGCCCCCGGCCAA [symbol] CTGGAGTGGATGGGACTGATCAACCCTTACACT |
| | GGCGGCACTGACTACAACCAGAAATTCCAAGGAAGGGTCACAATGACTGTGGACAA |
| | GAGCACATCCACTGCCTACATGGAGCTGAGCTCTCTGAGGAGCGAGGACACAGCCG |
| | TGTACTACTGTGCCAGAGAGAGACCCGGCGGCAGCCAGTTCATCTACTACTACCCTAT |
| | GGACTACTGGGGCCAAGGCACAACAGTGACTGTGAGCAGC |
| SEQ ID NO: 148 | GAGGTGCAACTGGTGGAGTCTGGGGGAGGCTTGGTCCAGCCTGGGGGGTCCCTGA |
| 9H9 AQQ-7B1 | GACTCTCCTGTGCAGCCTCTGGAGGCATCATTAGTACCTATTGGATGAGCTGGGTCC |
| H2L1 | GCCAGGCTCCAGGGAAGGGGCTGGAATGGGTGGCCAACATAAAGCAAGATGGAAG |
| | TGAGAAATATTATGTGGACTCTGTGAAGGGCCGATTCACCATCTCCAGAGACAACGC |
| | CAAGAACTCACTGTATTTGCAAATGAACAGCCTGAGAGTCGAGGACACGGCTATGT |
| | ATTACTGTGCGAGAGATCGTCCAGTGGCTGGTGCGTCGGCCCTCTGGGGCCAGGGA |
| | ACCCTGGTCACCGTCTCCTCAGCTAGCACAAAGGGCCCTTCCGTGTTTCCACTGGCTC |
| | CCAGCTCTAAGTCCACCAGCGGAGGAACAGCCGCTCTGGGCTGTCTGGTGAAGGAC |
| | TATTTCCCAGAGCCCGTGACCGTGAGCTGGAACTCTGGCGCCCTGACCAGCGGAGT |
| | GCATACATTTCCTGCTGTGCTGCAGTCCAGCGGCCTGTACTCTCTGTCTTCCGTGGTG |
| | ACCGTGCCAAGCTCTTCCCTGGGCACCCAGACATATATCTGCAACGTGAATCACAAG |
| | CCATCCAATACAAAGGTGGACAAGAAGGTGGAGCCCAAGAGCTGTGATAAGACCCA |
| | TACATGCCCCCCTTGTCCTGCTCCAGAG [symbol] GGAGGACCATCCGTGTTCCTGTTT |
| | CCACCCAAGCCTAAGGACACCCTGATGATCTCTAGGACCCCAGAGGTGACCTGCGTG |
| | GTGGTGGACGTGAGCCACGAGGATCCCGAGGTGAAGTTCAACTGGTACGTGGATG |
| | GCGTGGAGGTGCATAATGCCAAGACAAAGCCAAGGGAGGAGCAGTACAATAGCAC |
| | CTATCGGGTGGTGTCTGTGCTGACAGTGCTGCACCAGGACTGGCTGAACGGCAAGG |
| | AGTACAAGTGC [symbol] GTGTCTAATAAGGCCCTGCCCGCTCCTATCGAGAAGACCATCT |
| | CCAAGGCCAAGGGCCAGCCTAGGGAGCCACAGGTGTACACACTGCCTCCAAGCCGG |
| | GACGAGCTGACCAAGAACCAGGTGTCTCTGACATGTCTGGTGAAGGGCTTCTATCCC |
| | TCTGATATCGCTGTGGAGTGGGAGTCCAATGGCCAGCCTGAGAACAATTACAAGAC |
| | CACACCCCCTGTGCTGGACTCCGATGGCAGCTTCTTTCTGTATTCCAAGCTGACCGTG |
| | GATAAGAGCAGGTGGCAGCAGGGCAACGTGTTTTCTTGTTCCGTGATGCATGAGGC |
| | TCTGCACAATCATTACACACAGAAGAGCCTGTCTCTGTCCCCTGGCAAAGGCTCGAG |
| | CGGGGGAGGAGGTAGCCAAGTGCAGCTGGTGCAGAGCGGCGCTGAGGTGAAGAA |
| | GCCCGGCGCCAGCGTGAAAGTGAGCTGTAAGGCCTCCGGCTACAGCTTCACTGGCTA |
| | CACTATGCACTGGGTCAGACAAGCCCCCGGCCAA [symbol] CTGGAGTGGATGGGACTGA |
| | TCAACCCTTACACTGGCGGCACTGACTACAACCAGAAGTTCCAAGGAAGGGTGACTA |
| | TGACTGTGGATAGGTCCACAAGCACAGCCTACATGGAGCTGTCCTCTCTGAGATCCG |
| | AGGACACTGCCGTGTACTACTGTGCTAGGGAGAGACCCGGCGGCAGCCAGTTCATCT |
| | ACTACTACGCTCTGGACTACTGGGGCCAAGGCACAACAGTCACAGTGAGCAGCGGA |
| | GGGGGCGGTTCCGGAGGAGGCGGCAGCGGGGGAGGAGGTAGCGGCGGAGGTGG |
| | GTCTGACATCCAGATGACACAGAGCCCAAGCTCTCTGAGCGCCAGCGTGGGAGATA |
| | GGGTGACAATCACTTGTAGGGCCAGCGAGAACATCTACAGCTATCTGGCTTGGTACC |
| | AGCAGAAGCCCGGCAAGGCCCCAAAGTTTCTGGTGTACAACGCCGACACTCTCGCTG |
| | AGGGCGTCCCTTCTAGGTTTTCCGGCAGCGGCTCCGGCACTGACTTCACACTGACTAT |
| | CAGCTCTCTGCAGCCAGAGGATTTCGCCACATACTACTGCCAGCACCACTACGGCACT |
| | CCTTTCACATTCGGC [symbol] GGCACTAAGCTGGAGATCAAG |
| SEQ ID NO: 149 | GAGGTGCAACTGGTGGAGTCTGGGGGAGGCTTGGTCCAGCCTGGGGGGTCCCTGA |
| 9H9 AQQ-7B1 | GACTCTCCTGTGCAGCCTCTGGAGGCATCATTAGTACCTATTGGATGAGCTGGGTCC |
| L1H2 | GCCAGGCTCCAGGGAAGGGGCTGGAATGGGTGGCCAACATAAAGCAAGATGGAAG |
| | TGAGAAATATTATGTGGACTCTGTGAAGGGCCGATTCACCATCTCCAGAGACAACGC |
| | CAAGAACTCACTGTATTTGCAAATGAACAGCCTGAGAGTCGAGGACACGGCTATGT |
| | ATTACTGTGCGAGAGATCGTCCAGTGGCTGGTGCGTCGGCCCTCTGGGGCCAGGGA |
| | ACCCTGGTCACCGTCTCCTCAGCTAGCACAAAGGGCCCTTCCGTGTTTCCACTGGCTC |
| | CCAGCTCTAAGTCCACCAGCGGAGGAACAGCCGCTCTGGGCTGTCTGGTGAAGGAC |
| | TATTTCCCAGAGCCCGTGACCGTGAGCTGGAACTCTGGCGCCCTGACCAGCGGAGT |
| | GCATACATTTCCTGCTGTGCTGCAGTCCAGCGGCCTGTACTCTCTGTCTTCCGTGGTG |
| | ACCGTGCCAAGCTCTTCCCTGGGCACCCAGACATATATCTGCAACGTGAATCACAAG |
| | CCATCCAATACAAAGGTGGACAAGAAGGTGGAGCCCAAGAGCTGTGATAAGACCCA |
| | TACATGCCCCCCTTGTCCTGCTCCAGAG [symbol] GGAGGACCATCCGTGTTCCTGTTT |
| | CCACCCAAGCCTAAGGACACCCTGATGATCTCTAGGACCCCAGAGGTGACCTGCGTG |
| | GTGGTGGACGTGAGCCACGAGGATCCCGAGGTGAAGTTCAACTGGTACGTGGATG |
| | GCGTGGAGGTGCATAATGCCAAGACAAAGCCAAGGGAGGAGCAGTACAATAGCAC |
| | CTATCGGGTGGTGTCTGTGCTGACAGTGCTGCACCAGGACTGGCTGAACGGCAAGG |
| | AGTACAAGTGC [symbol] GTGTCTAATAAGGCCCTGCCCGCTCCTATCGAGAAGACCATCT |
| | CCAAGGCCAAGGGCCAGCCTAGGGAGCCACAGGTGTACACACTGCCTCCAAGCCGG |
| | GACGAGCTGACCAAGAACCAGGTGTCTCTGACATGTCTGGTGAAGGGCTTCTATCCC |
| | TCTGATATCGCTGTGGAGTGGGAGTCCAATGGCCAGCCTGAGAACAATTACAAGAC |
| | CACACCCCCTGTGCTGGACTCCGATGGCAGCTTCTTTCTGTATTCCAAGCTGACCGTG |
| | GATAAGAGCAGGTGGCAGCAGGGCAACGTGTTTTCTTGTTCCGTGATGCATGAGGC |
| | TCTGCACAATCATTACACACAGAAGAGCCTGTCTCTGTCCCCTGGCAAAGGCTCGAG |
| | CGGGGGAGGAGGTAGCGACATCCAGATGACACAGAGCCCAAGCTCTCTGAGCGCCA |
| | GCGTGGGAGATAGGGTGACAATCACTTGTAGGGCCAGCGAGAACATCTACAGCTAT |
| | CTGGCTTGGTACCAGCAGAAGCCCGGCAAGGCCCCAAAGTTTCTGGTGTACAACGCC |
| | GACACTCTCGCTGAGGGCGTCCCTTCTAGGTTTTCCGGCAGCGGCTCCGGCACTGAC |
| | TTCACACTGACTATCAGCTCTCTGCAGCCAGAGGATTTCGCCACATACTACTGCCAGC |
| | ACCACTACGGCACTCCTTTCACATTCGGC [symbol] GGCACTAAGCTGGAGATCAAGGGAG |
| | GGGGCGGTTCCGGAGGAGGCGGCAGCGGGGGAGGAGGTAGCGGCGGAGGTGGG |
| | TCTCAAGTGCAGCTGGTGCAGAGCGGCGCTGAGGTGAAGAAGCCCGGCGCCAGCGT |
| | GAAAGTGAGCTGTAAGGCCTCCGGCTACAGCTTCACTGGCTACACTATGCACTGGGT |
| | CAGACAAGCCCCCGGCCAA [symbol] CTGGAGTGGATGGGACTGATCAACCCTTACACTGG |
| | CGGCACTGACTACAACCAGAAGTTCCAAGGAAGGGTGACTATGACTGTGGATAGGT |
| | CCACAAGCACAGCCTACATGGAGCTGTCCTCTCTGAGATCCGAGGACACTGCCGTGT |
| | ACTACTGTGCTAGGGAGAGACCCGGCGGCAGCCAGTTCATCTACTACTACGCTCTGG |
| | ACTACTGGGGCCAAGGCACAACAGTCACAGTGAGCAGC |
| SEQ ID NO: 150 | GACATCCAGATGACCCAGTCTCCTTCCACCCTGTCTGCATCTGTAGGAGACAG |
| 9H9 LC | AGTCACCATCACTTGCCGGGCCAGTCAGAGTATTAGTGGCTGGTTGGCCTGGT |
| | ATCAGCAGAAACCAGGGAAAGCCCCTAAGCTCCTGATCTATAAGGCGTCTAGT |
| | TTAGAAAGTGGGGTCCCATCAAGGTTCAGCGGCAGTGGATCTGGGACAGAGTT |
| | CACTCTCACCATCAGCAGCCTGCAGCCTGATGATTTTGCAACTTATTACTGCCA |
| | ACAGTATTATGGTTCTTCTCGGACGTTCGGCCAAGGGACCAATGTGGAAATCA |
| | AACGTACGGTGGCCGCTCCAAGCGTGTTCATCTTTCCCCCTTCTGACGAGCAGC |
| | TGAAGTCTGGCACAGCCTCCGTGGTGTGCCTGCTGAACAACTTCTACCCCAGA |
| | GAGGCCAAGGTGCAGTGGAAGGTGGATAACGCTCTGCAGTCTGGCAATTCCCA |
| | GGAGAGCGTGACCGAGCAGGACTCTAAGGATTCCACATATAGCCTGAGCTCTA |
| | CCCTGACACTGTCTAAGGCCGATTACGAGAAGCACAAGGTGTATGCTTGCGAG |
| | GTGACCCATCAGGGCCTGTCCAGCCCAGTGACAAAGTCCTTCAATCGCGGCGA |
| | GTGT |

### EQUIVALENTS

Those skilled in the art will recognize, or be able to ascertain using no more than routine experimentation, many equivalents of the specific embodiments of the invention described herein. Such equivalents are intended to be encompassed by the following claims.

1. An isolated monoclonal antibody which binds to human ILT4, or antigen-binding fragment thereof, comprising heavy and light chain variable region CDR1, CDR2 and CDR3 amino acid sequences selected from the group consisting of:
(i) a heavy chain variable region CDR1 amino acid sequence selected from the consensus sequence: G Y T (I,M) H (SEQ ID NO: 21), or conservative sequence modifications thereof;
(ii) a heavy chain variable region CDR2 amino acid sequence as set forth in SEQ ID NO:3, or conservative sequence modifications thereof,
(iii) a heavy chain variable region CDR3 amino acid sequence selected from the consensus sequence: E R P G G S Q F I Y Y Y (P,A) (M,L) D Y (SEQ ID NO:22), or conservative sequence modifications thereof;
(iv) a light chain variable region CDR1 amino acid sequence selected from the consensus sequence: R A S (A,E) N I Y S Y L A (SEQ ID NO: 23), or conservative sequence modifications thereof;
(v) a light chain variable region CDR2 amino acid sequence selected from the consensus sequence: N A (I,D) T L A E (SEQ ID NO: 24), or conservative sequence modifications thereof;
(vi) a light chain variable region CDR3 amino acid sequence as set forth in SEQ ID NO:8, or conservative sequence modifications thereof.

2. An isolated monoclonal antibody which binds to human ILT4, or antigen-binding fragment thereof, comprising:
(a) heavy chain variable region CDR1, CDR2 and CDR3 amino acid sequences as set forth in SEQ ID NOs:1, 3, and 5, respectively, or conservative sequence modifications thereof, and light chain variable region CDR1, CDR2 and CDR3 amino acid sequences as set forth in SEQ ID NOs:6, 7, and 8, respectively, or conservative sequence modifications thereof,
(b) heavy chain variable region CDR1, CDR2 and CDR3 amino acid sequences as set forth in SEQ ID NOs:11, 13, and 15, respectively, or conservative sequence modifications thereof, and light chain variable region CDR1, CDR2 and CDR3 amino acid sequences as set forth in SEQ ID NOs:16, 17, and 18, respectively, or conservative sequence modifications thereof;
(c) a heavy chain variable region amino acid sequence as set forth in SEQ ID NO:9, or a sequence at least 80% identical thereto, and a light chain variable region amino acid sequence as set forth in SEQ ID NO:10, or a sequence at least 80% identical thereto;
(d) a heavy chain variable region amino acid sequence as set forth in SEQ ID NO:19, or a sequence at least 80% identical thereto, and a light chain variable region amino acid sequence as set forth in SEQ ID NO:20, or a sequence at least 80% identical thereto;
(e) a heavy chain amino acid sequence as set forth in SEQ ID NO:25, or a sequence at least 80% identical thereto, and a light chain amino acid sequence as set forth in SEQ ID NO:26, or a sequence at least 80% identical thereto;
(f) a heavy chain amino acid sequence as set forth in SEQ ID NO:27, or a sequence at least 80% identical thereto, and a light chain amino acid sequence as set forth in SEQ ID NO:28, or a sequence at least 80% identical thereto;
(g) a heavy chain variable region amino acid sequence selected from the group consisting of SEQ ID NO:9, 19, 97, 98, 99, 103, 104, 105, or a sequence at least 80% identical thereto; or
(h) a light chain variable region amino acid sequence selected from the group consisting of SEQ ID NO:10, 20, 100, 101, 102, 106, 107, 108, or a sequence at least 80% identical thereto.

3-4. (canceled)

5. The antibody, or antigen-binding fragment thereof, of claim 1, wherein the antibody, or antigen-binding fragment thereof, exhibits one or more of the following properties:
a. blocking ILT4 ligand (e.g., HLA-G ligand) binding to human ILT4;
b. enhancing or increasing cytokine or chemokine release by human macrophages;
c. potentiating the activation effects of LPS and IFNγ on macrophages;
d. promoting M1 macrophage polarization;
e. binding to human ILT4 with an equilibrium dissociation constant Kd of 10⁻⁹ M or less, or alternatively, an equilibrium association constant Ka of 10⁺⁹ M⁻¹ or greater;
f. lack of cross-reactivity with other ILT family members;
g. cross-reactivity with cynomolgus ILT4; and/or
h. inhibiting tumor cells that express ILT4.

6. The antigen-binding fragment thereof of claim 1, wherein the fragment is an Fab, Fab′, F(ab′)₂, Fv, or a single chain Fv.

7. A bispecific construct comprising the ILT4 antibody, or antigen binding fragment thereof, of claim 1 linked to a second binding agent.

8. The bispecific construct of claim 7, wherein the second binding agent binds to an immune checkpoint molecule, an immune costimulatory molecule, or a tumor antigen.

9. The bispecific construct of claim 8, wherein:
(a) the immune checkpoint molecule is PD-1, PD-L1 CTLA-4, LAG-3, TIGIT, TIM-3, VISTA, AXL, ILT2, or ILT3;
(b) the immune costimulatory molecule is CD27, CD40, 4-1BB, OX40, or GITR; or
(c) the tumor antigen is HER2, EGFR, ErB3, or CD24.

10-11. (canceled)

12. The bispecific construct of claim 8, wherein the binding agent binds to PD-L1 and comprises heavy and light chain CDR1, CDR2, and CDR3 amino acid sequences selected from the group consisting of:
(a) heavy chain variable region CDR1, CDR2 and CDR3 amino acid sequences as set forth in SEQ ID NOs: 59, 60, and 61, respectively, and light chain variable region CDR1, CDR2 and CDR3 amino acid sequences as set forth in SEQ ID NOs:62, 63, and 64, respectively;
(b) heavy chain variable region CDR1, CDR2 and CDR3 amino acid sequences as set forth in SEQ ID NOs: 35, 36, and 37, respectively, and light chain variable region CDR1, CDR2 and CDR3 amino acid sequences as set forth in SEQ ID NOs:38, 39, and 40, respectively;
(c) heavy chain variable region CDR1, CDR2 and CDR3 amino acid sequences as set forth in SEQ ID NOs: 41, 42, and 43, respectively, and light chain variable region CDR1, CDR2 and CDR3 amino acid sequences as set forth in SEQ ID NOs:44, 45, and 46, respectively;
(d) heavy chain variable region CDR1, CDR2 and CDR3 amino acid sequences as set forth in SEQ ID NOs: 47, 48, and 49, respectively, and light chain variable region CDR1, CDR2 and CDR3 amino acid sequences as set forth in SEQ ID NOs:50, 51, and 52, respectively;
(e) heavy chain variable region CDR1, CDR2 and CDR3 amino acid sequences as set forth in SEQ ID NOs: 53, 54, and 55, respectively, and light chain variable region CDR1, CDR2 and CDR3 amino acid sequences as set forth in SEQ ID NOs:56, 57, and 58, respectively; and
(f) heavy chain variable region CDR1, CDR2 and CDR3 amino acid sequences as set forth in SEQ ID NOs: 29, 30, and 31, respectively, and light chain variable region CDR1, CDR2 and CDR3 amino acid sequences as set forth in SEQ ID NOs:32, 33, and 34, respectively.

13. The bispecific construct of claim 12, wherein the PD-L1 binding agent comprises heavy and light chain variable region sequences selected from the group consisting of:
(a) a heavy chain variable region amino acid sequence as set forth in SEQ ID NO:87 and a light chain variable region amino acid sequence as set forth in SEQ ID NO: 88;
(b) a heavy chain variable region amino acid sequence as set forth in SEQ ID NO:79 and a light chain variable region amino acid sequence as set forth in SEQ ID NO: 80;
(c) a heavy chain variable region amino acid sequence as set forth in SEQ ID NO: 81 and a light chain variable region amino acid sequence as set forth in SEQ ID NO: 82;
(d) a heavy chain variable region amino acid sequence as set forth in SEQ ID NO: 83 and a light chain variable region amino acid sequence as set forth in SEQ ID NO:84;
(e) a heavy chain variable region amino acid sequence as set forth in SEQ ID NO:85 and a light chain variable region amino acid sequence as set forth in SEQ ID NO: 86; and
(f) a heavy chain variable region amino acid sequence as set forth in SEQ ID NO:77 and a light chain variable region amino acid sequence as set forth in SEQ ID NO: 78.

14. The bispecific construct of claim 7, wherein,
(a) the ILT4 antibody, or antigen binding fragment thereof, is an scFv; or
(b) the second antibody, or antigen binding fragment thereof, is an scFv.

15. (canceled)

16. The bispecific construct of claim 7, wherein the ILT4 antibody, or antigen binding fragment thereof, and the second binding agent comprise a human IgG1 constant domain.

17. The bispecific construct of claim 7, wherein (a) the ILT4 antibody, or antigen binding fragment thereof, is linked to the C-terminus of the heavy chain of the second binding agent or (b) the second binding agent is linked to the C-terminus of the heavy chain of the ILT4 antibody, or antigen binding fragment thereof.

18. The bispecific construct of claim 7, wherein the ILT4 antibody, or antigen binding fragment thereof, and the second binding agent are:
(a) genetically fused; or
(b) chemically conjugated.

19. (canceled)

20. A bispecific construct comprising an antibody which binds to human PD-L1, or antigen-binding fragment thereof, linked to an ILT4 scFv, wherein:
(a) the PD-L1 antibody, or antigen binding fragment thereof, comprises heavy chain variable region CDR1, CDR2 and CDR3 amino acid sequences as set forth in SEQ ID NOs: 59, 60, and 61, respectively, and light chain variable region CDR1, CDR2 and CDR3 amino acid sequences as set forth in SEQ ID NOs:62, 63, and 64, respectively; and
(b) the ILT4 scFv comprises:
(i) heavy chain variable region CDR1, CDR2 and CDR3 amino acid sequences as set forth in SEQ ID NOs:1, 3, and 5, respectively, and light chain variable region CDR1, CDR2 and CDR3 amino acid sequences as set forth in SEQ ID NOs:6, 7, and 8, respectively; or
(ii) heavy chain variable region CDR1, CDR2 and CDR3 amino acid sequences as set forth in SEQ ID NOs: 11, 13, and 15, respectively, and light chain variable region CDR1, CDR2 and CDR3 amino acid sequences as set forth in SEQ ID NOs:16, 17, and 18, respectively.

21. The bispecific construct of claim 20, wherein
(a) the PD-L1 antibody, or antigen binding fragment thereof, comprises a heavy chain variable region amino acid sequence as set forth in SEQ ID NO:87 and a light chain variable region amino acid sequence as set forth in SEQ ID NO:88; and
(b) the ILT4 scFv comprises:
(i) a heavy chain variable region amino acid sequence as set forth in SEQ ID NO:9 and a light chain variable region amino acid sequence as set forth in SEQ ID NO:10; or
(ii) a heavy chain variable region amino acid sequence as set forth in SEQ ID NO:19 and a light chain variable region amino acid sequence as set forth in SEQ ID NO:20.

22. The bispecific construct of claim 20, wherein the ILT4 scFv further comprises disulfide stabilization modifications with Cys substitutions at VH44 and VL100.

23. The bispecific construct of claim 20, wherein the PD-L1 antibody, or binding fragment thereof, comprises a human IgG1 constant domain.

24. A bispecific construct comprising an antibody which binds to human ILT4, or antigen-binding fragment thereof, linked to a PD-L1 scFv, wherein:
(a) the ILT4 antibody, or antigen binding fragment thereof, comprises:
(i) heavy chain variable region CDR1, CDR2 and CDR3 amino acid sequences as set forth in SEQ ID NOs:1, 3, and 5, respectively, and light chain variable region CDR1, CDR2 and CDR3 amino acid sequences as set forth in SEQ ID NOs:6, 7, and 8, respectively; or
(ii) heavy chain variable region CDR1, CDR2 and CDR3 amino acid sequences as set forth in SEQ ID NOs: 11, 13, and 15, respectively, and light chain variable region CDR1, CDR2 and CDR3 amino acid sequences as set forth in SEQ ID NOs:16, 17, and 18, respectively.
(b) the PD-L1 scFv comprises heavy chain variable region CDR1, CDR2 and CDR3 amino acid sequences as set forth in SEQ ID NOs: 59, 60, and 61, respectively, and light chain variable region CDR1, CDR2 and CDR3 amino acid sequences as set forth in SEQ ID NOs:62, 63, and 64, respectively.

25. The bispecific construct of claim 24, wherein
(a) the ILT4 antibody, or antigen binding fragment thereof, comprises:
(i) a heavy chain variable region amino acid sequence as set forth in SEQ ID NO:9 and a light chain variable region amino acid sequence as set forth in SEQ ID NO:10; or
(ii) a heavy chain variable region amino acid sequence as set forth in SEQ ID NO:19 and a light chain variable region amino acid sequence as set forth in SEQ ID NO:20; and
(b) the PD-L1 scFv comprises a heavy chain variable region amino acid sequence as set forth in SEQ ID NO:87 and a light chain variable region amino acid sequence as set forth in SEQ ID NO:88.

26. The bispecific construct of claim 24, wherein the ILT4 antibody, or binding fragment thereof, comprises a human IgG1 constant domain.

27. A multispecific construct comprising the bispecific construct of claim 7 and a third binding agent.

28-38. (canceled)

39. The bispecific construct of claim 7 which comprises a modified IgG1 domain with (i) a mutated human IgG1 Fc domain which comprises non-naturally occurring amino acids 234A, 235Q and 322Q as numbered by the EU index as set forth in Kabat.

40. The bispecific construct of claim 39 wherein the modified human IgG1 Fc domain further comprises non-naturally occurring amino acids 252Y, 254T and 256E as numbered by the EU index as set forth in Kabat.

41. A bispecific or multispecific antibody construct which comprises a modified human IgG1 Fc domain which comprises;
(a) non-naturally occurring amino acids 234A, 235Q and 322Q as numbered by the EU index as set forth in Kabat; and/or
(b) non-naturally occurring amino acids 252Y, 254T and 256E as numbered by the EU index as set forth in Kabat.

42. (canceled)

43. A composition comprising the antibody, or antigen binding fragment thereof, of claim 1.

44. A kit comprising the antibody, or antigen binding fragment thereof, of claim 1 and instructions for use.

45. An isolated nucleic acid molecule comprising a nucleotide sequence encoding the heavy and/or light chain variable regions of the antibody, or antigen binding fragment thereof, of claim 1.

46. An isolated nucleic acid molecule comprising a nucleotide sequence encoding the bispecific or multispecific construct of claim 41.

47. A vector comprising at least one nucleic acid molecule of claim 45.

48. A host cell comprising the vector of claim 47.

49. A method of activating macrophages comprising contacting macrophages with the antibody, or antigen binding fragment thereof, of claim 1.

50. A method for inducing or enhancing an immune response in a subject comprising administering to the subject the antibody, or antigen binding fragment thereof, of claim 1 in an amount effective to induce or enhance an immune response in the subject.

51. A method for treating cancer in a subject, the method comprising administering to the subject the antibody, or antigen binding fragment thereof, of claim 1 in an amount effective to treat the cancer.

52. The method of claim 51, wherein the cancer is selected from the group consisting of colorectal cancer, ovarian cancer, renal cell carcinoma, head and neck squamous cell carcinoma, breast cancer, lung cancer, bladder cancer, prostate cancer, melanoma, gynecological cancers, sarcoma, lymphoma, and glioblastoma.

53. A method of treating a tumor in a subject, the method comprising administering to the subject the antibody, or antigen binding fragment thereof, of claim 1 in an amount effective to treat the tumor.

54-58. (canceled)
